(19) United States
(12) Patent Application Publication
(10) Pub. No.: US 2026/0317426 A1
(43) Pub. Date: Oct. 15, 2026

(54) METHODS OF TREATING CANCER WITH ANTI-MICA/B ANTIBODIES

(71) Applicant: CULLINAN MICA CORPORATION, CAMBRIDGE, MA (US)

(72) Inventors: JOHN EDWARD JANIK, CAMBRIDGE, MA (US); GUANGYU LIU, CAMBRIDGE, MA (US); IRINA MICHAILOVNA SHAPIRO, CAMBRIDGE, MA (US); KERRY WHALEN, CAMBRIDGE, MA (US); JEFFREY ALAN JONES, CAMBRIDGE, MA (US); DIVYA GUPTA, CAMBRIDGE, MA (US)

(21) Appl. No.: 19/482,528

(22) PCT Filed: May 22, 2024
(86) PCT No.: PCT/US2024/030464

§ 371 Date: Nov. 7, 2025

Related U.S. Application Data
(60) Provisional application No. 63/504,163, filed on May 24, 2023.
(60) Provisional application No. 63/505,705, filed on Jun. 1, 2023.
(60) Provisional application No. 63/593,609, filed on Oct. 27, 2023.
(60) Provisional application No. 63/559,422, filed on Feb. 29, 2024.
(60) Provisional application No. 63/574,800, filed on Apr. 4, 2024.

(51) Int. Cl. C07K 16/28; A61K 31/573; A61K 39/00; A61K 45/06; A61P 35/00

(52) U.S. Cl. CPC C07K 16/2833; A61K 31/573; A61K 45/06; A61P 35/00; C07K 16/2818; A61K 2039/505; A61K 2039/545; C07K 2317/24; C07K 2317/31; C07K 2317/565; C07K 2317/732; C07K 2317/76; C07K 2317/92

(57) ABSTRACT

Provided herein are methods of treating cancer with antibodies or antigen-binding fragments thereof that specifically bind to MICA/B having heavy chain, light chain, variable heavy chain domains (VH), variable light chain domains (VL), and complementarity determining regions (CDRs) disclosed herein.

### RELATED APPLICATIONS

The present patent application claims the priority benefit of U.S. Provisional Patent Application Ser. No. 63/504,163, filed May 24, 2023; U.S. Provisional Patent Application Ser. No. 63/505,705, filed Jun. 1, 2023; U.S. Provisional Patent Application Ser. No. 63/593,609, filed Oct. 27, 2023; U.S. Provisional Patent Application Ser. No. 63/559,422, filed Feb. 29, 2024; and U.S. Provisional Patent Application Ser. No. 63/574,800, filed Apr. 4, 2024 the content of each is hereby incorporated by reference in its entirety into this disclosure.

### REFERENCE TO AN ELECTRONIC SEQUENCE LISTING

The contents of the electronic sequence listing (67239WO01_SequenceListing.xml; Size: 14,820 bytes; and Date of Creation: May 13, 2024) is herein incorporated by reference in its entirety.

### BACKGROUND

The field of immuno-oncology has revolutionized cancer patient care and improved survival and quality of life for patients. However, to date, much of the field has focused on exploiting the power of the adaptive immune response through therapeutic targeting of T cells. While these approaches have markedly advanced the field, some challenges remain. Moreover, the clinical benefit of T cell therapies does not extend to all patients or tumor indications, particularly in the solid tumor setting. Alternative strategies, such as engaging the innate immune system, have become an intense area of focus in the field. In particular, the engagement of natural killer (NK) cells as potent effectors of the innate immune response has emerged as a promising modality in immunotherapy. Whalen et al., 2023 provide a detailed review of the potential of engaging NK cells for cancer therapy using NKG2D, CD16A, and other cell surface receptors (Whalen et al., mAbs 15:12208697).

In contrast to T cells, natural killer (NK) cells act as a first line of defense and have the ability to detect and clear tumor or virally infected cells without any requirement for prior antigen-specific activation or differentiation. As such, NK cells possess several attractive properties justifying the development of agents that harness their therapeutic potential. The intricate balance between activating versus inhibitory signals, which is a hallmark of NK cells, calls for the engagement of multiple activating receptors, a combination of receptor agonists with inhibitors of NK cell checkpoints, and/or combination with T cell therapies, in order to maximize therapeutic efficacy in patients. Like T cells, NK cells are increasingly recognized as having high cytotoxic potential when appropriately engaged. When equipped with a strong activating receptor specific for a cell surface antigen, NK cell therapies have been shown to drive complete responses in patients. Likewise, a T cell receptor complex (TCR) in NK cells enhanced their effector function in a similar manner to T-cells.

NK cell therapies have several advantages over T cell therapies. Most notable is their potential for allogenic, off-the-shelf use as they are able to avoid graft-versus-host reactivity due to the lack of a TCR. In addition, NK cell therapies are typically associated with less severe toxicities, including immune effector cell-associated neurotoxicity syndrome (ICANs) and cytokine release syndrome (CRS), as compared to certain T cell-based therapies. However, there remain several key challenges for NK cell-based cell therapies, including inefficient transgene delivery, NK cell manufacturing complexities, exhaustion and limited persistence of genetically engineered NK cells.

Cancer therapy has indeed been revolutionized by the development of antibodies the specifically target the T cell rather than the cancer cell in order to effectively treat and in some cases, provide durable responses, in cancer patients. Unlike cell therapies, mAbs that engage NK cells, much like those targeting T cells, have the potential to reach every effector NK cell, rely on standardized mAb manufacturing methods, and enable predictable pharmacokinetic properties, thereby providing a potentially powerful and pan-cancer response. The field is replete with mAb-based therapies that target the interaction between the CD16A/Fc-gamma receptor IIIA (FcγRIIIa) expressed on NK cells and the Fc gamma domain 1 (Fcγ1) of mAbs that recognize tumor-associated cell surface antigens on cancer cells. Many chimeric, human, and humanized IgG1 antibody therapeutics, such as rituximab (targeting CD20), daratumumab (CD38), and trastuzumab (human epidermal growth factor receptor 2-HER2), engage CD16A. However, these prior antibody-based therapies leave another key NK cell-activating receptor, namely NKG2D, untapped. NKG2D is a potent modulator of NK cell activation that has recently emerged as an important target in the immune oncology field. NKG2D recognizes eight distinct ligands that are upregulated on tumor cells: MHC class I chain-related protein A (MICA), MHC class I chain-related protein sequence B (MICB), and UL-16-binding proteins (ULBPs) named ULBP1 through ULBP6

While strategies to therapeutically harness the activity of NKG2D are still in early development, compared to other NKG2D ligands, MICA is the most consistently and highly expressed across all solid tumors and hematological malignancies. The second broadest expression pattern is exhibited by MICB, which is closely related in sequence to MICA. This makes both MICA and MICB (MICA/B) attractive pan-cancer targets.

MICA and MICB are stress-induced surface glycoproteins that are upregulated in a wide variety of human tumors. Engagement of MICA/B by the activating receptor NKG2D on NK cells and several subsets of T cells results in immune-mediated target cell lysis.

However, cancer cells avoid NKG2D-mediated NK cell lysis by exploiting the unique alpha 3 region of MICA/B, which is the target for proteolytic shedding of the extracellular domain (ECD) from the cancer cell surface through the action of multiple proteases that most cancer cells express on their cell surface or release into the tumor microenvironment. There is a need for therapies that prevent MICA/B shedding thereby preserving the MICA/B on the tumor cell surface to promote NKG2D mediated NK cell lysis of tumor cells. Such therapies would have potential in treating a diverse array of cancers. There is thus an unmet need to develop new therapies that are capable of treating cancers that use this mechanism to evade the immune response.

### SUMMARY

Disclosed herein, are methods of treating cancer with antibodies that specifically bind to MICA/B.

Accordingly, in a first aspect, the disclosure features a method of treating endometrial cancer in a subject, comprising administering to the subject a therapeutically effective amount of an antibody that binds to MICA/B (an anti-MICA/B antibody).

In some embodiments of the first aspect, the subject has previously been treated with a PD-1 inhibitor. In some embodiments, the subject has previously been treated with a VEGF inhibitor or hormonal therapy. In some embodiments, the subject has not previously been treated with a PD-1 inhibitor. In some embodiments, the PD-1 inhibitor is an anti-PD-1 antibody. In some embodiments, the anti-PD-1 inhibitor comprises pimivalimab, pembrolizumab, nivolumab, cemiplimab, AMP-224, APM-514, or spartalizumab.

In some embodiments of the first aspect, the endometrial cancer is HER2+. In some embodiments, the endometrial cancer is one or both of ER+ or PR+. In some embodiments, the cancer is mutant for one or more of: EGFR, TP53, HER2, PTEN, ARIDIA, CTNNB1, or PLK3R1.

In some embodiments of the first aspect, after administration of the anti-MICA/B antibody, the subject has a response. In some embodiments, the response is a complete response. In some embodiments, the response is a partial response. In some embodiments, after administration of the anti-MICA/B antibody, the subject has stable disease. In some embodiments, the response or stable disease after administration of the anti-MICA/B antibody is at least 1, 2, 3, 4, 5, 6, 7, 8, 9, 10, 11, 12, 18, 24, 30, 36, 42, 48, 54, 60 months or more.

In some embodiments of the first aspect, the subject has previously been treated with a VEGF inhibitor, an EGFR inhibitor, and/or a hormone therapy.

In some embodiments of the first aspect, the EGFR inhibitor comprises a small molecule tyrosine kinase inhibitor (TKI). In some embodiments, the TKI is gefitinib, erlotinib, afatinib, dacomitinib, or osimertinib). In some embodiments, the EGFR inhibitor comprises an anti-EGFR antibody. In some embodiments, the anti-EGFR antibody is cetuximab, panitumumab, or necitumumab.

In some embodiments of the first aspect, the VEGF inhibitor comprises a small molecule tyrosine kinase inhibitor (TKI). In some embodiments, the TKI is sunitinib, sorafenib, axitinib, pazopanib, or lenvatinib. In some embodiments, the VEGF inhibitor comprises an anti-VEGF antibody. In some embodiments, the anti-VEGF antibody is bevacizumab or ranibizumab.

In some embodiments of the first aspect, the hormone therapy comprises an aromatase inhibitor (AI). In some embodiments, the AI is anastrozole. In some embodiments, the hormone therapy comprises a selective estrogen receptor modulator (SERM). In some embodiments, the hormone therapy comprises a leuteinizing hormone-releasing hormone (LHRH) agonist. In some embodiments, the hormone therapy comprises an anti-androgen. In some embodiments, the hormone therapy comprises a CYP17 inhibitor. In some embodiments, the hormone therapy comprises a progestin. In some embodiments, the hormone therapy comprises an adrenolytic. In some embodiments, the hormone therapy comprises an estrogen receptor antagonist.

In some embodiments of the first aspect, the subject has a cancer characterized by a low level of PD-L1 expression. In some embodiments, the subject has a cancer characterized by a high level of PD-L1 expression. In some embodiments, the subject has a cancer characterized by a low tumor mutation burden (TMB). In some embodiments, the subject has a cancer characterized by a high tumor mutation burden (TMB). In some embodiments, the subject has an immunologically cold cancer. In some embodiments, the subject has an immunologically hot cancer. In some embodiments, the subject has a hormone-sensitive cancer. In some embodiments, the subject has a cancer characterized by an overexpression of oncogenic drivers. In some embodiments, the subject has a cancer that expresses one or more of: EGFR, ER, PR, or HER2.

In some embodiments of the first aspect, the subject has a cancer that has metastasized.

In some embodiments of the first aspect, the subject has previously received a therapy comprising a chemotherapy.

In some embodiments of the first aspect, the anti-MICA/B antibody is administered at a dose of about 3 mg/kg to about 10 mg/kg. In some embodiments, the anti-MICA/B antibody is administered to the subject according to a dosing interval. In some embodiments, the dosing interval is referred to as a cycle. In some embodiments, the dosing interval comprises a three-week cycle, and wherein the anti-MICA/B antibody is administered once every three weeks (Q3W). In some embodiments, the dosing interval is repeated at least 1 time, at least 2 times, at least 3 times, at least 4 times, at least 5 times, at least 6 times, at least 7 times, at least 8 times, at least 9 times, or at least 10 times. In some embodiments, the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, or 5 years.

In some embodiments of the first aspect, the anti-MICA/B antibody comprises a light chain variable region (VL) comprising a light chain complementarity determining region 1 (LCDR1) of SEQ ID NO: 1, a light chain complementarity determining region (LCDR2) of SEQ ID NO: 2, a light chain complementarity determining region 3 (LCDR3) of SEQ ID NO: 3 and a heavy chain variable region (VH) comprising a heavy chain complementarity determining region 1 (HCDR1) of SEQ ID NO: 4, a heavy chain complementarity determining region 2 (HCDR2) of SEQ ID NO: 5, and a heavy chain complementarity determining region 3 (HCDR3) of SEQ ID NO: 6. In some embodiments, the VL comprises the amino acid sequence of SEQ ID NO: 7, or an amino acid sequence having at least about 85%, 90%, 95%, or 99% sequence identity thereto. In some embodiments, the VH comprises the amino acid sequence of SEQ ID NO: 8, or an amino acid sequence having at least about 85%, 90%, 95%, or 99% sequence identity thereto. In some embodiments, the light chain comprises the amino acid sequence of SEQ ID NO: 9, or an amino acid sequence having at least about 85%, 90%, 95%, or 99% sequence identity thereto. In some embodiments, the heavy chain comprises the amino acid sequence of SEQ ID NO: 10, or an amino acid sequence having at least about 85%, 90%, 95%, or 99% sequence identity thereto.

In some embodiments of the first aspect, the anti-MICA/B antibody specifically binds to a MICA protein, a MICB protein, or both MICA and MICB protein. In some embodiments, the anti-MICA/B antibody binds to an alpha-3 domain of a MICA protein, a MICB protein, or both MICA and MICB protein. In some embodiments, the MICA protein is membrane-bound MICA protein, soluble MICA protein, or both. In some embodiments, the MICB protein is membrane-bound MICB protein, soluble MICB protein, or both.

In some embodiments of the first aspect, the anti-MICA/B antibody is selected from a whole immunoglobulin, an scFv, a Fab, a F(ab′) 2, or a disulfide linked Fv. In some embodiments, the anti-MICA/B antibody is an IgG or IgM. In some embodiments, the anti-MICA/B antibody is a humanized or a chimeric antibody.

In some embodiments of the first aspect, the method further comprises administering to the subject an effective amount of a PD-1 inhibitor. In some embodiments, the PD-1 inhibitor is an anti-PD-1 antibody. In some embodiments, the anti-PD-1 inhibitor comprises pimivalimab, pembrolizumab, nivolumab, cemiplimab, AMP-224, APM-514, or spartalizumab.

In a second aspect, the disclosure features a method of treating parotid cancer in a subject, comprising administering to the subject a therapeutically effective amount of an antibody that binds to MICA/B (an anti-MICA/B antibody).

In some embodiments of the second aspect, the parotid cancer overexpresses one or both of EGFR and HER2.

In some embodiments of the second aspect, the parotid cancer is a mucoepidermoid parotid cancer.

In some embodiments of the second aspect, the cancer is mutant for one or more of: EGFR, TP53, HER2, PTEN, ARIDIA, CTNNB1, or PLK3R1.

In some embodiments of the second aspect, after administration of the anti-MICA/B antibody, the subject has a response. In some embodiments, the response is a complete response. In some embodiments, the response is a partial response. In some embodiments, after administration of the anti-MICA/B antibody, the subject has stable disease. In some embodiments, the response or stable disease after administration of the anti-MICA/B antibody is at least 1, 2, 3, 4, 5, 6, 7, 8, 9, 10, 11, 12, 18, 24, 30, 36, 42, 48, 54, 60 months or more.

In some embodiments of the second aspect, the subject has previously been treated with a VEGF inhibitor, an EGFR inhibitor, and/or a hormone therapy.

In some embodiments of the second aspect, the EGFR inhibitor comprises a small molecule tyrosine kinase inhibitor (TKI). In some embodiments, the TKI is gefitinib, erlotinib, afatinib, dacomitinib, or osimertinib). In some embodiments, the EGFR inhibitor comprises an anti-EGFR antibody. In some embodiments, the anti-EGFR antibody is cetuximab, panitumumab, or necitumumab.

In some embodiments of the second aspect, the VEGF inhibitor comprises a small molecule tyrosine kinase inhibitor (TKI). In some embodiments, the TKI is sunitinib, sorafenib, axitinib, pazopanib, or lenvatinib. In some embodiments, the VEGF inhibitor comprises an anti-VEGF antibody. In some embodiments, the anti-VEGF antibody is bevacizumab or ranibizumab.

In some embodiments of the second aspect, the hormone therapy comprises an aromatase inhibitor (AI). In some embodiments, the AI is anastrozole. In some embodiments, the hormone therapy comprises a selective estrogen receptor modulator (SERM). In some embodiments, the hormone therapy comprises a leuteinizing hormone-releasing hormone (LHRH) agonist. In some embodiments, the hormone therapy comprises an anti-androgen. In some embodiments, the hormone therapy comprises a CYP17 inhibitor. In some embodiments, the hormone therapy comprises a progestin. In some embodiments, the hormone therapy comprises an adrenolytic. In some embodiments, the hormone therapy comprises an estrogen receptor antagonist.

In some embodiments of the second aspect, the subject has a cancer characterized by a low level of PD-L1 expression. In some embodiments, the subject has a cancer characterized by a high level of PD-L1 expression. In some embodiments, the subject has a cancer characterized by a low tumor mutation burden (TMB). In some embodiments, the subject has a cancer characterized by a high tumor mutation burden (TMB). In some embodiments, the subject has an immunologically cold cancer. In some embodiments, the subject has an immunologically hot cancer. In some embodiments, the subject has a hormone-sensitive cancer. In some embodiments, the subject has a cancer characterized by an overexpression of oncogenic drivers. In some embodiments, the subject has a cancer that expresses one or more of: EGFR, ER, PR, or HER2.

In some embodiments of the second aspect, the subject has a cancer that has metastasized.

In some embodiments of the second aspect, the subject has previously received a therapy comprising a chemotherapy.

In some embodiments of the second aspect, the anti-MICA/B antibody is administered at a dose of about 3 mg/kg to about 10 mg/kg. In some embodiments, the anti-MICA/B antibody is administered to the subject according to a dosing interval. In some embodiments, the dosing interval is referred to as a cycle. In some embodiments, the dosing interval comprises a three-week cycle, and wherein the anti-MICA/B antibody is administered once every three weeks (Q3W). In some embodiments, the dosing interval is repeated at least 1 time, at least 2 times, at least 3 times, at least 4 times, at least 5 times, at least 6 times, at least 7 times, at least 8 times, at least 9 times, or at least 10 times. In some embodiments, the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, or 5 years.

In some embodiments of the second aspect, the anti-MICA/B antibody comprises a light chain variable region (VL) comprising a light chain complementarity determining region 1 (LCDR1) of SEQ ID NO: 1, a light chain complementarity determining region (LCDR2) of SEQ ID NO: 2, a light chain complementarity determining region 3 (LCDR3) of SEQ ID NO: 3 and a heavy chain variable region (VH) comprising a heavy chain complementarity determining region 1 (HCDR1) of SEQ ID NO: 4, a heavy chain complementarity determining region 2 (HCDR2) of SEQ ID NO: 5, and a heavy chain complementarity determining region 3 (HCDR3) of SEQ ID NO: 6. In some embodiments, the VL comprises the amino acid sequence of SEQ ID NO: 7, or an amino acid sequence having at least about 85%, 90%, 95%, or 99% sequence identity thereto. In some embodiments, the VH comprises the amino acid sequence of SEQ ID NO: 8, or an amino acid sequence having at least about 85%, 90%, 95%, or 99% sequence identity thereto. In some embodiments, the light chain comprises the amino acid sequence of SEQ ID NO: 9, or an amino acid sequence having at least about 85%, 90%, 95%, or 99% sequence identity thereto. In some embodiments, the heavy chain comprises the amino acid sequence of SEQ ID NO: 10, or an amino acid sequence having at least about 85%, 90%, 95%, or 99% sequence identity thereto.

In some embodiments of the second aspect, the anti-MICA/B antibody specifically binds to a MICA protein, a MICB protein, or both MICA and MICB protein. In some embodiments, the anti-MICA/B antibody binds to an alpha-3 domain of a MICA protein, a MICB protein, or both MICA and MICB protein. In some embodiments, the MICA protein is membrane-bound MICA protein, soluble MICA protein, or both. In some embodiments, the MICB protein is membrane-bound MICB protein, soluble MICB protein, or both.

In some embodiments of the second aspect, the anti-MICA/B antibody is selected from a whole immunoglobulin, an scFv, a Fab, a F(ab′)2, or a disulfide linked Fv. In some embodiments, the anti-MICA/B antibody is an IgG or IgM. In some embodiments, the anti-MICA/B antibody is a humanized or a chimeric antibody.

In some embodiments of the second aspect, the method further comprises administering to the subject an effective amount of a PD-1 inhibitor. In some embodiments, the PD-1 inhibitor is an anti-PD-1 antibody. In some embodiments, the anti-PD-1 inhibitor comprises pimivalimab, pembrolizumab, nivolumab, cemiplimab, AMP-224, APM-514, or spartalizumab.

In a third aspect, the disclosure features a method of treating cancer in a subject, the method comprising administering to the subject a therapeutically effective amount of an antibody that binds to MICA/B (an anti-MICA/B antibody), wherein the subject has previously been treated with: (i) a VEGF inhibitor; (ii) an EGFR inhibitor; and/or (iii) a hormone therapy.

In some embodiments of the third aspect, the subject responded to the VEGF inhibitor, the EGFR inhibitor or the hormone therapy. In some embodiments, the response is a complete response. In some embodiments, the response is a partial response. In some embodiments, the subject had a period of response of 1, 2, 3, 4, 5, 6, 7, 8, 9, 10, 11, 12, 18, 24, 30, 36, 42, 48, 54, 60 months or more.

In some embodiments of the third aspect, the subject progressed following a period of response to the VEGF inhibitor, the EGFR inhibitor, or the hormone therapy. In some embodiments, the administration of the anti-MICA/B antibody is after the progression following the period of response to the VEGF inhibitor, the EGFR inhibitor, or the hormone therapy.

In some embodiments of the third aspect, the subject had stable disease upon administration of the VEGF inhibitor, the EGFR inhibitor or the hormone therapy. In some embodiments, the subject had a period of stable disease of 1, 2, 3, 4, 5, 6, 7, 8, 9, 10, 11, 12, 18, 24, 30, 36, 42, 48, 54, 60 months or more.

In some embodiments of the third aspect, treatment with the VEGF inhibitor, the EGFR inhibitor, or the hormone therapy ceased before initiation of administration of the anti-MICA/B antibody.

In some embodiments of the third aspect, treatment with the VEGF inhibitor, the EGFR inhibitor, or the hormone therapy continues after initiation of administration of the anti-MICA/B antibody.

In some embodiments of the third aspect, the subject progressed upon treatment with the VEGF inhibitor, the EGFR inhibitor, or the hormone therapy.

In some embodiments of the third aspect, after administration of the anti-MICA/B antibody, the subject has a response. In some embodiments, the response is a complete response. In some embodiments, the response is a partial response. In some embodiments, after administration of the anti-MICA/B antibody, the subject has stable disease. In some embodiments, the response or stable disease after administration of the anti-MICA/B antibody is at least 1, 2, 3, 4, 5, 6, 7, 8, 9, 10, 11, 12, 18, 24, 30, 36, 42, 48, 54, 60 months or more.

In some embodiments of the third aspect, the EGFR inhibitor comprises a small molecule tyrosine kinase inhibitor (TKI). In some embodiments, the TKI is gefitinib, erlotinib, afatinib, dacomitinib, or osimertinib). In some embodiments, the EGFR inhibitor comprises an anti-EGFR antibody. In some embodiments, the anti-EGFR antibody is cetuximab, panitumumab, or necitumumab.

In some embodiments of the third aspect, the VEGF inhibitor comprises a small molecule tyrosine kinase inhibitor (TKI). In some embodiments, the TKI is sunitinib, sorafenib, axitinib, pazopanib, or lenvatinib. In some embodiments, the VEGF inhibitor comprises an anti-VEGF antibody. In some embodiments, the anti-VEGF antibody is bevacizumab or ranibizumab.

In some embodiments of the third aspect, the hormone therapy comprises an aromatase inhibitor (AI). In some embodiments, the AI is anastrozole. In some embodiments, the hormone therapy comprises a selective estrogen receptor modulator (SERM). In some embodiments, the hormone therapy comprises a leuteinizing hormone-releasing hormone (LHRH) agonist. In some embodiments, the hormone therapy comprises an anti-androgen. In some embodiments, the hormone therapy comprises a CYP17 inhibitor. In some embodiments, the hormone therapy comprises a progestin. In some embodiments, the hormone therapy comprises an adrenolytic. In some embodiments, the hormone therapy comprises an estrogen receptor antagonist.

In some embodiments of the third aspect, the cancer is a tumor of the female genital tract. In some embodiments, the tumor of the female genital tract comprises an endometrial tumor. In some embodiments, the tumor of the female genital tract comprises an ovarian cancer, or a cervical cancer. In some embodiments, the cancer is a salivary gland tumor. In some embodiments, the salivary gland tumor comprises a mucoepidermoid tumor. In some embodiments, the mucoepidermoid tumor is a parotid tumor.

In some embodiments of the third aspect, the cancer is a breast cancer. In some embodiments, the cancer is a prostate cancer. In some embodiments, the cancer is a lung cancer. In some embodiments, the cancer is or a colon cancer. In some embodiments, the subject has a parotid gland cancer. In some embodiments, the subject has cervical cancer. In some embodiments, the subject has an endometrial cancer. In some embodiments, the subject has a breast cancer. In some embodiments, the subject has a colon cancer. In some embodiments, the subject has an ovarian cancer. In some embodiments, the subject has a prostate cancer. In some embodiments, the subject has a sarcoma. In some embodiments, the subject has a melanoma. In some embodiments, the subject has an adenoid cystic salivary tumor. In some embodiments, the subject has a peritoneal mesothelioma. In some embodiments, the subject has a squamous cell carcinoma of the rectum. In some embodiments, the subject has a leiomyosarcoma. In some embodiments, the subject has a colorectal cancer. In some embodiments, the subject has a kidney cancer. In some embodiments, the subject has a thyroid cancer. In some embodiments, the subject has an NSCLC. In some embodiments, the subject has a duodenum cancer. In some embodiments, the subject has a pancreatic cancer. In some embodiments, the subject has a mediatinal intimal sarcoma. In some embodiments, the subject has a head and neck cancer. In some embodiments, the subject has a caecal cancer.

In some embodiments of the third aspect, the subject has a cancer characterized by a low level of PD-L1 expression. In some embodiments, the subject has a cancer characterized by a high level of PD-L1 expression. In some embodiments, the subject has a cancer characterized by a low tumor mutation burden (TMB). In some embodiments, the subject has a cancer characterized by a high tumor mutation burden (TMB). In some embodiments, the subject has an immunologically cold cancer. In some embodiments, the subject has an immunologically hot cancer. In some embodiments, the subject has a hormone-sensitive cancer. In some embodiments, the subject has a cancer characterized by an overexpression of oncogenic drivers. In some embodiments, the subject has a cancer that expresses one or more of: EGFR, ER, PR, or HER2.

In some embodiments of the third aspect, the subject has a cancer that has metastasized.

In some embodiments of the third aspect, the subject has previously received a therapy comprising a chemotherapy.

In some embodiments of the third aspect, the anti-MICA/B antibody is administered at a dose of about 3 mg/kg to about 10 mg/kg. In some embodiments, the anti-MICA/B antibody is administered to the subject according to a dosing interval. In some embodiments, the dosing interval is referred to as a cycle. In some embodiments, the dosing interval comprises a three-week cycle, and wherein the anti-MICA/B antibody is administered once every three weeks (Q3W). In some embodiments, the dosing interval is repeated at least 1 time, at least 2 times, at least 3 times, at least 4 times, at least 5 times, at least 6 times, at least 7 times, at least 8 times, at least 9 times, or at least 10 times. In some embodiments, the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, or 5 years.

In some embodiments of the third aspect, the anti-MICA/B antibody comprises a light chain variable region (VL) comprising a light chain complementarity determining region 1 (LCDR1) of SEQ ID NO: 1, a light chain complementarity determining region (LCDR2) of SEQ ID NO: 2, a light chain complementarity determining region 3 (LCDR3) of SEQ ID NO: 3 and a heavy chain variable region (VH) comprising a heavy chain complementarity determining region 1 (HCDR1) of SEQ ID NO: 4, a heavy chain complementarity determining region 2 (HCDR2) of SEQ ID NO: 5, and a heavy chain complementarity determining region 3 (HCDR3) of SEQ ID NO: 6. In some embodiments, the VL comprises the amino acid sequence of SEQ ID NO: 7, or an amino acid sequence having at least about 85%, 90%, 95%, or 99% sequence identity thereto. In some embodiments, the VH comprises the amino acid sequence of SEQ ID NO: 8, or an amino acid sequence having at least about 85%, 90%, 95%, or 99% sequence identity thereto. In some embodiments, the light chain comprises the amino acid sequence of SEQ ID NO: 9, or an amino acid sequence having at least about 85%, 90%, 95%, or 99% sequence identity thereto. In some embodiments, the heavy chain comprises the amino acid sequence of SEQ ID NO: 10, or an amino acid sequence having at least about 85%, 90%, 95%, or 99% sequence identity thereto.

In some embodiments of the third aspect, the anti-MICA/B antibody specifically binds to a MICA protein, a MICB protein, or both MICA and MICB protein. In some embodiments, the anti-MICA/B antibody binds to an alpha-3 domain of a MICA protein, a MICB protein, or both MICA and MICB protein. In some embodiments, the MICA protein is membrane-bound MICA protein, soluble MICA protein, or both. In some embodiments, the MICB protein is membrane-bound MICB protein, soluble MICB protein, or both.

In some embodiments of the third aspect, the anti-MICA/B antibody is selected from a whole immunoglobulin, an scFv, a Fab, a F(ab′)2, or a disulfide linked Fv. In some embodiments, the anti-MICA/B antibody is an IgG or IgM. In some embodiments, the anti-MICA/B antibody is a humanized or a chimeric antibody.

In some embodiments of the third aspect, the method further comprises administering to the subject an effective amount of a PD-1 inhibitor. In some embodiments, the PD-1 inhibitor is an anti-PD-1 antibody. In some embodiments, the anti-PD-1 inhibitor comprises pimivalimab, pembrolizumab, nivolumab, cemiplimab, AMP-224, APM-514, or spartalizumab.

In a fourth aspect, the disclosure features a method of treating cancer in a subject, the method comprising administering to the subject a therapeutically effective amount of an antibody that binds to MICA/B (an anti-MICA/B antibody), wherein the subject has previously been treated with a PD-1 inhibitor. In some embodiments, the subject responded to the PD-1 inhibitor.

In some embodiments of the fourth aspect, the response was a complete response. In some embodiments, the response was a partial response. In some embodiments, the subject had a period of response of 1, 2, 3, 4, 5, 6, 7, 8, 9, 10, 11, 12, 18, 24, 30, 36, 42, 48, 54, 60 months or more. In some embodiments, the subject progressed following a period of response to the PD-1 inhibitor.

In some embodiments of the fourth aspect, the subject had stable disease upon administration of the PD-1 inhibitor. In some embodiments, the subject had a period of stable disease of 1, 2, 3, 4, 5, 6, 7, 8, 9, 10, 11, 12, 18, 24, 30, 36, 42, 48, 54, 60 months or more.

In some embodiments of the fourth aspect, treatment with the PD-1 inhibitor ceased before initiation of administration of the anti-MICA/B antibody.

In some embodiments of the fourth aspect, treatment with the PD-1 inhibitor continues after initiation of administration of the anti-MICA/B antibody.

In some embodiments of the fourth aspect, after administration of the anti-MICA/B antibody, the subject has a response. In some embodiments, the response is a complete response. In some embodiments, the response is a partial response. In some embodiments, after administration of the anti-MICA/B antibody, the subject has stable disease. In some embodiments, the response or stable disease after administration of the anti-MICA/B antibody is at least 1, 2, 3, 4, 5, 6, 7, 8, 9, 10, 11, 12, 18, 24, 30, 36, 42, 48, 54, 60 months or more.

In some embodiments of the fourth aspect, the PD-1 inhibitor is an anti-PD-1 antibody. In some embodiments, the anti-PD-1 inhibitor comprises pimivalimab, pembrolizumab, nivolumab, cemiplimab, AMP-224, APM-514, or spartalizumab.

In some embodiments of the fourth aspect, the subject has previously been treated with a VEGF inhibitor, an EGFR inhibitor, and/or a hormone therapy.

In some embodiments of the fourth aspect, the EGFR inhibitor comprises a small molecule tyrosine kinase inhibitor (TKI). In some embodiments, the TKI is gefitinib, erlotinib, afatinib, dacomitinib, or osimertinib). In some embodiments, the EGFR inhibitor comprises an anti-EGFR antibody. In some embodiments, the anti-EGFR antibody is cetuximab, panitumumab, or necitumumab.

In some embodiments of the fourth aspect, the VEGF inhibitor comprises a small molecule tyrosine kinase inhibitor (TKI). In some embodiments, the TKI is sunitinib, sorafenib, axitinib, pazopanib, or lenvatinib. In some embodiments, the VEGF inhibitor comprises an anti-VEGF antibody. In some embodiments, the anti-VEGF antibody is bevacizumab or ranibizumab.

In some embodiments of the fourth aspect, the hormone therapy comprises an aromatase inhibitor (AI). In some embodiments, the AI is anastrozole. In some embodiments, the hormone therapy comprises a selective estrogen receptor modulator (SERM). In some embodiments, the hormone therapy comprises a leuteinizing hormone-releasing hormone (LHRH) agonist. In some embodiments, the hormone therapy comprises an anti-androgen. In some embodiments, the hormone therapy comprises a CYP17 inhibitor. In some embodiments, the hormone therapy comprises a progestin. In some embodiments, the hormone therapy comprises an adrenolytic. In some embodiments, the hormone therapy comprises an estrogen receptor antagonist.

In some embodiments of the fourth aspect, the cancer is a tumor of the female genital tract. In some embodiments, the tumor of the female genital tract comprises an endometrial tumor. In some embodiments, the tumor of the female genital tract comprises an ovarian cancer, or a cervical cancer. In some embodiments, the cancer is a salivary gland tumor. In some embodiments, the salivary gland tumor comprises a mucoepidermoid tumor. In some embodiments, the mucoepidermoid tumor is a parotid tumor.

In some embodiments of the fourth aspect, the cancer is a breast cancer. In some embodiments, the cancer is a prostate cancer. In some embodiments, the cancer is a lung cancer. In some embodiments, the cancer is or a colon cancer. In some embodiments, the subject has a parotid gland cancer. In some embodiments, the subject has cervical cancer. In some embodiments, the subject has an endometrial cancer. In some embodiments, the subject has a breast cancer. In some embodiments, the subject has a colon cancer. In some embodiments, the subject has an ovarian cancer. In some embodiments, the subject has a prostate cancer. In some embodiments, the subject has a sarcoma. In some embodiments, the subject has a melanoma. In some embodiments, the subject has an adenoid cystic salivary tumor. In some embodiments, the subject has a peritoneal mesothelioma. In some embodiments, the subject has a squamous cell carcinoma of the rectum. In some embodiments, the subject has a leiomyosarcoma. In some embodiments, the subject has a colorectal cancer. In some embodiments, the subject has a kidney cancer. In some embodiments, the subject has a thyroid cancer. In some embodiments, the subject has an NSCLC. In some embodiments, the subject has a duodenum cancer. In some embodiments, the subject has a pancreatic cancer. In some embodiments, the subject has a mediatinal intimal sarcoma. In some embodiments, the subject has a head and neck cancer. In some embodiments, the subject has a caecal cancer.

In some embodiments of the fourth aspect, the subject has a cancer characterized by a low level of PD-L1 expression. In some embodiments, the subject has a cancer characterized by a high level of PD-L1 expression. In some embodiments, the subject has a cancer characterized by a low tumor mutation burden (TMB). In some embodiments, the subject has a cancer characterized by a high tumor mutation burden (TMB). In some embodiments, the subject has an immunologically cold cancer. In some embodiments, the subject has an immunologically hot cancer. In some embodiments, the subject has a hormone-sensitive cancer. In some embodiments, the subject has a cancer characterized by an overexpression of oncogenic drivers. In some embodiments, the subject has a cancer that expresses one or more of: EGFR, ER, PR, or HER2.

In some embodiments of the fourth aspect, the subject has a cancer that has metastasized.

In some embodiments of the fourth aspect, the subject has previously received a therapy comprising a chemotherapy.

In some embodiments of the fourth aspect, the anti-MICA/B antibody is administered at a dose of about 3 mg/kg to about 10 mg/kg. In some embodiments, the anti-MICA/B antibody is administered to the subject according to a dosing interval. In some embodiments, the dosing interval is referred to as a cycle. In some embodiments, the dosing interval comprises a three-week cycle, and wherein the anti-MICA/B antibody is administered once every three weeks (Q3W). In some embodiments, the dosing interval is repeated at least 1 time, at least 2 times, at least 3 times, at least 4 times, at least 5 times, at least 6 times, at least 7 times, at least 8 times, at least 9 times, or at least 10 times. In some embodiments, the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, or 5 years.

In some embodiments of the fourth aspect, the anti-MICA/B antibody comprises a light chain variable region (VL) comprising a light chain complementarity determining region 1 (LCDR1) of SEQ ID NO: 1, a light chain complementarity determining region (LCDR2) of SEQ ID NO: 2, a light chain complementarity determining region 3 (LCDR3) of SEQ ID NO: 3 and a heavy chain variable region (VH) comprising a heavy chain complementarity determining region 1 (HCDR1) of SEQ ID NO: 4, a heavy chain complementarity determining region 2 (HCDR2) of SEQ ID NO: 5, and a heavy chain complementarity determining region 3 (HCDR3) of SEQ ID NO: 6. In some embodiments, the VL comprises the amino acid sequence of SEQ ID NO: 7, or an amino acid sequence having at least about 85%, 90%, 95%, or 99% sequence identity thereto. In some embodiments, the VH comprises the amino acid sequence of SEQ ID NO: 8, or an amino acid sequence having at least about 85%, 90%, 95%, or 99% sequence identity thereto. In some embodiments, the light chain comprises the amino acid sequence of SEQ ID NO: 9, or an amino acid sequence having at least about 85%, 90%, 95%, or 99% sequence identity thereto. In some embodiments, the heavy chain comprises the amino acid sequence of SEQ ID NO: 10, or an amino acid sequence having at least about 85%, 90%, 95%, or 99% sequence identity thereto.

In some embodiments of the fourth aspect, the anti-MICA/B antibody specifically binds to a MICA protein, a MICB protein, or both MICA and MICB protein. In some embodiments, the anti-MICA/B antibody binds to an alpha-3 domain of a MICA protein, a MICB protein, or both MICA and MICB protein. In some embodiments, the MICA protein is membrane-bound MICA protein, soluble MICA protein, or both. In some embodiments, the MICB protein is membrane-bound MICB protein, soluble MICB protein, or both.

In some embodiments of the fourth aspect, the anti-MICA/B antibody is selected from a whole immunoglobulin, an scFv, a Fab, a F(ab′)2, or a disulfide linked Fv. In some embodiments, the anti-MICA/B antibody is an IgG or IgM. In some embodiments, the anti-MICA/B antibody is a humanized or a chimeric antibody.

In some embodiments of the fourth aspect, the method further comprises administering to the subject an effective amount of a PD-1 inhibitor. In some embodiments, the PD-1 inhibitor is an anti-PD-1 antibody. In some embodiments, the anti-PD-1 inhibitor comprises pimivalimab, pembrolizumab, nivolumab, cemiplimab, AMP-224, APM-514, or spartalizumab.

In a fifth aspect, the disclosure features a method of treating cancer in a subject, comprising (i) administering to the subject first dose of an antibody that binds to MICA/B (an anti-MICA/B antibody), and (ii) administering to the subject a therapeutically effective amount of a corticosteroid prior to the first dose of the anti-MICA/B antibody.

In some embodiments of the fifth aspect, the corticosteroid is administered 30-60 minutes prior to administration of the anti-MICA/B antibody. In some embodiments, the corticosteroid is administered at a dose of between about 2 mg to about 50 mg. In some embodiments, the corticosteroid is administered at a dose of about 10 mg. In some embodiments, the corticosteroid is administered orally or intravenously.

In some embodiments of the fifth aspect, the method further comprises administering one or more subsequent doses of the anti-MICA/B antibody.

In some embodiments of the fifth aspect, the corticosteroid is administered prior to the first dose of the anti-MICA/B antibody but is not administered prior to the one or more subsequent doses of the anti-MICA/B antibody.

In some embodiments of the fifth aspect, the corticosteroid is dexamethasone.

In some embodiments of the fifth aspect, the cancer is a tumor of the female genital tract. In some embodiments, the tumor of the female genital tract comprises an endometrial tumor. In some embodiments, the tumor of the female genital tract comprises an ovarian cancer, or a cervical cancer. In some embodiments, the cancer is a salivary gland tumor. In some embodiments, the salivary gland tumor comprises a mucoepidermoid tumor. In some embodiments, the mucoepidermoid tumor is a parotid tumor.

In some embodiments of the fifth aspect, the cancer is a breast cancer. In some embodiments, the cancer is a prostate cancer. In some embodiments, the cancer is a lung cancer. In some embodiments, the cancer is or a colon cancer. In some embodiments, the subject has a parotid gland cancer. In some embodiments, the subject has cervical cancer. In some embodiments, the subject has an endometrial cancer. In some embodiments, the subject has a breast cancer. In some embodiments, the subject has a colon cancer. In some embodiments, the subject has an ovarian cancer. In some embodiments, the subject has a prostate cancer. In some embodiments, the subject has a sarcoma. In some embodiments, the subject has a melanoma. In some embodiments, the subject has an adenoid cystic salivary tumor. In some embodiments, the subject has a peritoneal mesothelioma. In some embodiments, the subject has a squamous cell carcinoma of the rectum. In some embodiments, the subject has a leiomyosarcoma. In some embodiments, the subject has a colorectal cancer. In some embodiments, the subject has a kidney cancer. In some embodiments, the subject has a thyroid cancer. In some embodiments, the subject has an NSCLC. In some embodiments, the subject has a duodenum cancer. In some embodiments, the subject has a pancreatic cancer. In some embodiments, the subject has a mediatinal intimal sarcoma. In some embodiments, the subject has a head and neck cancer. In some embodiments, the subject has a caecal cancer.

In some embodiments of the fifth aspect, the subject has a cancer characterized by a low level of PD-L1 expression. In some embodiments, the subject has a cancer characterized by a high level of PD-L1 expression. In some embodiments, the subject has a cancer characterized by a low tumor mutation burden (TMB). In some embodiments, the subject has a cancer characterized by a high tumor mutation burden (TMB). In some embodiments, the subject has an immunologically cold cancer. In some embodiments, the subject has an immunologically hot cancer. In some embodiments, the subject has a hormone-sensitive cancer. In some embodiments, the subject has a cancer characterized by an overexpression of oncogenic drivers. In some embodiments, the subject has a cancer that expresses one or more of: EGFR, ER, PR, or HER2.

In some embodiments of the fifth aspect, the subject has a cancer that has metastasized.

In some embodiments, of the fifth aspect the subject has previously received a therapy comprising a chemotherapy.

In some embodiments of the fifth aspect, the anti-MICA/B antibody is administered at a dose of about 3 mg/kg to about 10 mg/kg. In some embodiments, the anti-MICA/B antibody is administered to the subject according to a dosing interval. In some embodiments, the dosing interval is referred to as a cycle. In some embodiments, the dosing interval comprises a three-week cycle, and wherein the anti-MICA/B antibody is administered once every three weeks (Q3W). In some embodiments, the dosing interval is repeated at least 1 time, at least 2 times, at least 3 times, at least 4 times, at least 5 times, at least 6 times, at least 7 times, at least 8 times, at least 9 times, or at least 10 times. In some embodiments, the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, or 5 years.

In some embodiments of the fifth aspect, the anti-MICA/B antibody comprises a light chain variable region (VL) comprising a light chain complementarity determining region 1 (LCDR1) of SEQ ID NO: 1, a light chain complementarity determining region (LCDR2) of SEQ ID NO: 2, a light chain complementarity determining region 3 (LCDR3) of SEQ ID NO: 3 and a heavy chain variable region (VH) comprising a heavy chain complementarity determining region 1 (HCDR1) of SEQ ID NO: 4, a heavy chain complementarity determining region 2 (HCDR2) of SEQ ID NO: 5, and a heavy chain complementarity determining region 3 (HCDR3) of SEQ ID NO: 6. In some embodiments, the VL comprises the amino acid sequence of SEQ ID NO: 7, or an amino acid sequence having at least about 85%, 90%, 95%, or 99% sequence identity thereto. In some embodiments, the VH comprises the amino acid sequence of SEQ ID NO: 8, or an amino acid sequence having at least about 85%, 90%, 95%, or 99% sequence identity thereto. In some embodiments, the light chain comprises the amino acid sequence of SEQ ID NO: 9, or an amino acid sequence having at least about 85%, 90%, 95%, or 99% sequence identity thereto. In some embodiments, the heavy chain comprises the amino acid sequence of SEQ ID NO: 10, or an amino acid sequence having at least about 85%, 90%, 95%, or 99% sequence identity thereto.

In some embodiments of the fifth aspect, the anti-MICA/B antibody specifically binds to a MICA protein, a MICB protein, or both MICA and MICB protein. In some embodiments, the anti-MICA/B antibody binds to an alpha-3 domain of a MICA protein, a MICB protein, or both MICA and MICB protein. In some embodiments, the MICA protein is membrane-bound MICA protein, soluble MICA protein, or both. In some embodiments, the MICB protein is membrane-bound MICB protein, soluble MICB protein, or both.

In some embodiments of the fifth aspect, the anti-MICA/B antibody is selected from a whole immunoglobulin, an scFv, a Fab, a F(ab′) 2, or a disulfide linked Fv. In some embodiments, the anti-MICA/B antibody is an IgG or IgM. In some embodiments, the anti-MICA/B antibody is a humanized or a chimeric antibody.

In some embodiments of the fifth aspect, the method further comprises administering to the subject an effective amount of a PD-1 inhibitor. In some embodiments, the PD-1 inhibitor is an anti-PD-1 antibody. In some embodiments, the anti-PD-1 inhibitor comprises pimivalimab, pembrolizumab, nivolumab, cemiplimab, AMP-224, APM-514, or spartalizumab.

One aspect of the disclosure is a method of treating multiple myeloma in a subject, the method comprising administering to the subject a therapeutically effective amount of an antibody that binds to MICA/B (an anti-MICA/B antibody).

In some embodiments, the method further comprises administering to said subject a therapeutically effective amount of an immunomodulatory drug. In some embodiments, the method further comprises administering to said subject a therapeutically effective amount of dexamethasone.

In some embodiments, after administration of the anti-MICA/B antibody, the subject has a response (e.g., a complete response or partial response) or stable disease. In some embodiments, said subject has relapsed/refractory multiple myeloma. In some embodiments, said subject is non-responsive to corticosteroids, melphalan, or a combination of vincristine, doxorubicin, dexamethasone.

In some embodiments, the method further comprises administering to the subject a therapeutically effective amount of a corticosteroid prior to the first dose of the anti-MICA/B antibody. In some embodiments, the corticosteroid is administered 30-60 minutes prior to administration of the anti-MICA/B antibody. In some embodiments, the corticosteroid is administered at a dose of between about 2 mg to about 50 mg. In some embodiments, the corticosteroid is administered at a dose of about 10 mg. In some embodiments, the corticosteroid is administered orally or intravenously.

In some embodiments, the method further comprises administering one or more subsequent doses of the anti-MICA/B antibody. In some embodiments, the corticosteroid is administered prior to the first dose of the anti-MICA/B antibody but is not administered prior to the one or more subsequent doses of the anti-MICA/B antibody. In some embodiments, the corticosteroid is dexamethasone.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will be obtained by reference to the following detailed description that sets forth illustrative embodiments, in which the principles of the invention are utilized, and the accompanying drawings of which:

FIGS. 1A-1C depict binding characteristics of CLN-619. FIG. 1A shows the mean of three independent experiments of CLN-619 binding to representative MICA allelic variants and the canonical MICB allelic variant as measured by Octet. FIG. 1B shows the mean of two independent experiments of CLN-619 binding to representative MICA allelic variants and the canonical MICB allelic variant as measured by ELISA. (N=2). CLN-619 had similar levels of binding to the various proteins, therefore not all symbols are visible. (c) CLN-619 binding to 28 of the most common MICA allelic variants as measured by Luminex in two independent experiments. 6D4 was utilized as a positive control (PMID: 28334733). Error bars represent SEM.

FIG. 2 depicts a CLN-619 epitope. X-ray crystal structures of the human Fab antibody fragment CLN-619 in complex with the antigen MICA1 alpha 3 domain at 2.12 Å resolution. The binding interface of CLN-619 is a discontinuous epitope comprising 19 amino acid residues exclusively in the alpha 3 domain of MICA.

FIGS. 3A and 3B illustrate that CLN-619 modulates cell surface MICA/B through inhibition of shedding. FIG. 3A are graphs showing cell surface levels of MICA/B that were measured by flow cytometry. FIG. 3B are graphs showing soluble MICA/B in the cell supernatant that was measured by ELISA (N=3 or N=2; error bars represent SEM). Geometric mean EC50 values and 95% confidence intervals are reported.

FIGS. 4A and 4B illustrate that CLN-619 mediates NK activation and cell killing of MICA/B expressing target cells. FIG. 4A are graphs from an ELISA which was used to measure IFNγ in cell supernatants collected from 96 hr co-culture killing assay with NK cells and MICA/B expressing targets cells (E:T 40:1) in the presence of serially titrated antibody. N=3. CLN-619 DANA is a mutated (D265A/N297A),Fc-silenced version of CLN-619. FIG. 4B are graphs from a xCELLigence platform that was utilized to measure MICA/B expressing target cell death from experiment described in FIG. 4A. Representative curves are shown.

FIG. 5 illustrates that CLN-619 induces ADCC. MICA-expressing target cells treated with serially titrated antibody were co-cultured with NFAT-luciferase Jurkat cells expressing the human FcγRIIIa (CD16A) high affinity (V158) variant or low affinity (F158) variant at an E:T ratio of 10:1. Luciferase activity was quantified after 6 hrs. Geometric mean EC50 values and 95% confidence intervals are reported. E:T control background subtracted.

FIG. 6 illustrates that CLN-619 promotes antibody dependent phagocytosis. Macrophage phagocytosis of fluorescently labeled target cells was measured by flow cytometry. Representative curves for treatment of HCC1534 and HCT-116 cells with CLN-619 in two donors. Cetuximab and isotype control antibody treatment was carried out at the highest concentration. Geometric mean EC50 values 95% confidence intervals are reported. E:T control background subtracted. Error bars represent SEM.

FIGS. 7A-7C illustrate that CLN-619 treatment results in potent anti-tumor effects in preclinical mouse models. FIG. 7A is a graph depicting the results of BALB/c SCID mice that were inoculated with PLC/PRF/5 human liver cancer cells (N=10/group). Mice were treated 3× weekly i.p with CLN-619 at two dose levels. Statistics represent one-way ANOVA multiple comparisons. * P=0.015. FIG. 7B is a graph depicting the results of BALB/c SCID mice that were inoculated with HCC1534 human lung cancer cells (N=10/group). Mice were treated 2× weekly i.p. with CLN-619 or CLN-619 Fc silenced at a range of doses. Statistics represent one-way ANOVA multiple comparisons. NS-not significant. FIG. 7C is a graph depicting the results of BALB/c SCID mice that were inoculated with HCT-116 luciferase tagged human colon cancer cells (N=10/group). Mice were treated 2× weekly i.p. with CLN-619 or hIgG1. Survival analysis was done on Day 41 using Kaplan Meier.

FIG. 8 is a table showing allelic frequencies of MICA allelic variants tested in luminex assay. Allelic frequencies are based on high throughput sequencing of over 2 million donor samples as previously described [16]. MICA*009 could not be distinguished from MICA*049, and MICA*001 is the canonical allele for MICA.

FIG. 9 is a table showing CLN-619 binding to FcR's. Kinetic Binding Parameters Determined by Biacore-SPR. A Biacore chip was coupled with the various his-tagged FcR's via an anti-HIS antibody, and test antibody was run over the bound FcR. Kinetic analysis was done using Biacore software. Relative scale for affinity (M): ++++10-8 to −9; +++10-7; ++10-6; +10-5; +/−detectable binding; —no detectable binding.

FIG. 10 is a table showing the sequence analysis of the CLN-619 epitope for MICA/B alleles. Alleles listed are >1% frequency in the population and have high homology with conservative amino acid changes shown in orange. * Denotes canonical alleles.

FIG. 11 are graphs illustrating that CLN-619 does not interfere with MICA-NKG2D binding. Primary NK cells were incubated with His-tagged MICA ECD in the presence of CLN-619 or hIgG1 control antibody without (a) or with anti_NKG2D antibody. The amount of MICA bound to the cell surface was measured by flow cytometry.

FIG. 12 is a graph illustrating that CLN-619 DANA retains the ability to modulate cell surface MICA/B. Cell surface levels of MICA/B were measured by flow cytometry.

FIGS. 13A and 13B depict pharmacokinetic parameters following a single IP injection of CLN-619 in BALB/c SCID Mice. FIG. 13A is a graph depicting concentration-time profiles and pharmacokinetic parameters shown in FIG. 13B for CLN-619 following a single IP dose administration of 1, 3, and 10 mg/kg. Mean and standard deviation are reported.

FIG. 14 is a table illustrating baseline characteristics of patients enrolled in a phase 1 open-label dose escalation study of CLN-619 for the treatment of advanced solid tumors.

FIG. 15 is a schematic illustrating the structure of the dose-escalation study, including the number of patients within each dose-level cohort.

FIG. 16 is a table summarizing treatment-emergent adverse events (TEAE) observed in ≥10% of patients.

FIGS. 17A and 17B are a graph and table showing mean serum concentration of CLN-619 at various time points post-administration and PK parameters stratified by dosage level.

FIG. 18 is a series of graphs showing cytokine expression in the longitudinal serum samples from patients treated with CLN-619 monotherapy tested using Myriad RBM Inflammation and custom MAPs on the Luminex platform. Graphs depict average absolute cytokine levels (pg/ml) per dose level over time. Error bars represent SD.

FIGS. 19A and 19B are graphs showing time on treatment and clinical activity for each study patient receiving the CLN-619 monotherapy.

FIGS. 20A and 20B are tables summarizing objective response summary and best monotherapy response for study patients receiving the CLN-619 monotherapy.

FIG. 21 is a series of images from a study patient having a mucoepidermoid parotid cancer at baseline (top panel), at cycle 4 of CLN-619 treatment (middle panel), and at cycle 7 of CLN-619 treatment (bottom panel).

FIG. 22 are images from a study patient having endometrial cancer at baseline (left panel) and at cycle 10 of CLN-619 treatment (right panel).

FIGS. 23A and 23B show the overall study design for the Clinical Study of CLN-619 alone and in combination with Pembrolizumab in patients with advanced solid tumors.

FIG. 24 shows the schedule of events for the Clinical Study of CLN-619 alone and in combination with Pembrolizumab in patients with advanced solid tumors.

FIG. 25 shows the sampling schedule for the Clinical Study of CLN-619 alone and in combination with Pembrolizumab in patients with advanced solid tumors.

FIG. 26A shows CLN-619-001 infusion time instructions.

FIG. 26B shows CLN-619-001 infusion time instructions for Module D (Loading Dose Cohort).

FIG. 27 shows time on treatment and clinical activity for combination cohorts.

FIG. 28 shows time on treatment and clinical activity for patients with NSCLC.

FIG. 29 shows time on treatment and clinical activity for monotherapy cohorts ≥1 mg/kg.

FIG. 30 is an overview of treatment-related adverse events (TEAEs).

FIG. 31 shows the overall study design for use of CLN-619 for the treatment of R/R multiple myeloma.

FIGS. 32A and 32B show the effect of CLN-619 on T cell activation. FIG. 32A is a graph from a xCELLigence platform that was used to measure cytolysis in a 72 hr co-culture killing assay of CD8+ T cells negatively selected from PBMCs combined with MICA/B expressing targets cells (E:T 10:1) in the presence of serially titrated antibody. FIG. 32B is a graph from flow cytometry utilized to measure the percent of CD8+ T cells that express CD25 from the experiment described in FIG. 32A. FIG. 32C shows the effect of CLN-619 on T cell cytolysis. ELISA was used to measure IFNγ in cell supernatants collected from experiment described in FIG. 32A. FIGS. 32A-32C N=3. Representative curves are shown.

### DETAILED DESCRIPTION

Disclosed herein, in some embodiments, are monoclonal antibodies that bind specifically to MICA/B. In some embodiments, MICA/B antibodies herein bind to MICA/B proteins or fragments thereof and modulate immune response in an individual, thereby treating cancer (e.g. hepatocellular carcinoma).

Major histocompatibility complex class I-related chain A and B (MICA/B) are two stress-inducible ligands for natural killer cell (NK) receptor NKG2D and play an important role in mediating the cytotoxicity of NK and T cells. Soluble MICA/B shed by diseased cells (e.g. cancer cells) desensitizes NK and T cells through binding of NKG2D receptor, thereby suppressing the immune response. Accordingly, modulation of MICA/B is useful in modulating an immune response in an individual, for example, in an individual suffering from cancer.

The generation of therapeutics that inhibit shedding of MICA/B is an emerging strategy to leverage these NKG2D ligands. Disclosed herein are clinical data demonstrating the clinical safety and efficacy of targeting inhibition of MICA/B shedding by use of CLN-619, a humanized IgG1 antibody that is currently the only MICA/B-targeting mAb in the clinic. CLN-619 prevents shedding of MICA/B from cancer cells by binding to the alpha 3 domain and contains an active Fcγ1 domain to drive ADCC and ADCP. The MICA/B genes are highly polymorphic, nevertheless CLN-619 has shown surprising and unexpectedly broad reactivity to all allelic variants tested. Reduced levels of shed MICA and a concomitant increase in MICA on the surface of tumor cells has been observed with CLN-619 treatment. Importantly, the alpha 1 and alpha 2 domains of MICA, which bind to NKG2D, are unencumbered by the bound antibody. In fact, CLN-619 enhances the binding of MICA to NKG2D, which appears to be dependent on the Fcγ1 domain of the antibody that can concomitantly bind to CD16A on NK cells. Notably, CLN-619 shows compelling single-agent activity at low doses in tumor xenograft models, where its activity critically relies upon a functional Fcγ1 domain. As such, CLN-619 promises to present therapeutic efficacy as a single agent or in combination with other agents that engage other NK cell receptors and/or one or more checkpoint inhibitors.

Stabilization of MICA/B by CLN-619 leads to accumulation of these NKG2D ligands on the tumor cell surface, thereby overcoming immune evasion by MICA/B shedding. Without being bound to a particular theory or mechanism of action, this likely maximizes ADCC and ADCP functions of the antibody due to increased target antigen density. Importantly, CLN-619 has the potential to activate NK cells via simultaneous and perhaps synergistic engagement of two key activating receptors: NKG2D and CD16A. Cooperation between the two receptors is supported by published data showing engagement NKG2D can lower the activation threshold of several receptors including CD16A. Cooperation between the two receptors may be important when considering NK cell-activating receptors in the TME are often downmodulated. The disclosure herein shows important data from clinical trials of CLN-619 in patients treated with CLN-619 alone or in combination with pembrolizumab in patients with advanced solid tumors (Phase 1 clinical trial no. NCT05117476).

Monotherapy activity is uncommon in oncology phase 1 studies, as evidenced a retrospective analysis showing objective response rates in the low single digits among more than 4000 patients enrolled in solid tumor phase 1 studies sponsored by the US National Cancer Institute between 2000-2019. The monotherapy response rates for CLN-619 in evaluable patients compare favorably to both the historical NCI data as well as the TIGIT and lag-3 antibodies and are truly encouraging this early in the CLN-619 program.

Further details of clinical trials of CLN-619 in patients treated with CLN-619 alone or in combination with pembrolizumab in patients with advanced solid tumors can be found in the appendix.

One such study is a Phase 1 dose escalation and expansion study testing both CLN-619 monotherapy and CLN-619 in combination with pembrolizumab in staggered, parallel cohorts. CLN-619 monotherapy was dosed every 3 weeks. Pre-medication was required prior to the first infusion only and mandated to include corticosteroids starting at the 3 mg/mg dose level. Dose-limiting toxicity was evaluated during the first cycle and the response is evaluated every 3 cycles according to RECIST criteria.

The monotherapy arm employed a standard 3×3 dose escalation testing doses from 0.1 to 10 mg/mg. Dose levels cleared for DLT could be further expanded (sometimes called “backfill”) up to a maximum of 10 patients treated in total per dose once a dose level was cleared. A total of 37 patients were treated-their median age was 63 and 62% were female. This heavily pre-treated group received a median 3 prior therapies, and just over half had prior CPI treatment.

RECIST responses were seen at doses starting at 3 mg/kg, including an ongoing durable CR in a parotid gland patient treated at 3 mg/kg as well as 2 PRs in endometrial patients treated at 3 and 10 mg/kg. Both the parotid CR and one of the endometrial PRs were in patients who had progressed during prior checkpoint inhibitor therapy. At doses from 1 mg/kg and above, 10 among 22 response evaluable patients achieved a best response of objective response or stable disease.

In addition to the 2 endometrial PRs, 3 cervical patients achieved stable disease and 2 ovarian patients also achieved stable disease. The data shows that of the 10 patients enrolled in the study with gynecologic cancers, 7 achieved a best response of objective response or stable disease.

Durable stable disease was also seen in one breast cancer and one salivary gland cancer patient, underscoring that objective monotherapy clinical activity has been observed across a range of tumor types.

In the scans from a durable CR patient with the parotid tumor, biopsy-proven lymph node recurrence met criteria for complete response at C4D1, was confirmed at C7D1, and was ongoing at the time of data cutoff more than 9 months after beginning treatment with CLN-619. Notably, this patient experienced a 30-month sustained PR to checkpoint inhibitor therapy prior to progressing during treatment.

In scans from one of the two endometrial patients who experienced a confirmed PR, baseline images demonstrate multiple bilateral pulmonary metastases that had regressed to meet criteria for PR at C4D1 and confirmed by the subsequent scan shown at C7D1. This patient had progressed following prior therapy with platinum-based chemotherapy+Herceptin, anastrazole, doxorubicin, and most recently 9 months of treatment with pembrolizumab+lenvantinib.

Treatment emerging adverse events in >10% of patients demonstrates that most AEs observed during CLN-619 monotherapy are grade ½. Notably, no dose limiting toxicities were observed at any dose level assessed. A single grade 3 treatment related AE of laryngeal edema, a type of infusion-related adverse event observed with other monoclonal antibodies, occurred during the first infusion in a patient that did not receive mandated steroid premedication. All other IRRs were grade 1 or 2 and occurred only in cycle one. The data supports that CLN-619 monotherapy was well tolerated and as such, the safety profile should support ease of combinability with other agents.

The data further demonstrates that CLN-619 monotherapy was well tolerated with no DLTs up to 10 mg/kg and that CLN-619 has shown notable monotherapy activity, including confirmed and ongoing objective responses, across a variety of tumor types, including patients whose disease has progressed during checkpoint inhibitor treatment.

Given the broad tumor expression of the pathway, it is contemplated that CLN-619 will likely have pan-cancer therapeutic activity.

The data shown herein in the examples demonstrate the safety and efficacy of CLN-619 monotherapy showing acceptable safety and promising clinical activity, including objective responses in multiple tumor types, in both check-point experienced and check-point naïve patients. Of particular note, there was notable single-agent activity in multiple gynecological malignancies, e.g., endometrial and cervical cancers. This clinical activity in multiple gynecologic cancer types proves the potential of CLN-619 in patients with high unmet need. More specifically, monotherapy dose escalation demonstrates acceptable safety profile of CLN-619 across all doses assessed (0.1, 0.3, 1, 3, 6, and 10 mg/kg), with no dose limiting toxicities observed. Consistent with other therapeutic monoclonal antibodies, infusion-related reactions were all Grade ½ and limited to the first dose in patients receiving mandated pre-medication. Importantly, the CLN-619 therapy was effective against multiple tumor types at the doses administered, including demonstration of stable disease being observed in patients' tumors as disparate as cervical, ovarian, salivary gland, and breast cancers. Certain patients were heavily pre-treated with a median of 3 prior systemic therapies (1-7), including 54% previously receiving immune checkpoint inhibitor. First clinical data for CLN-619 monotherapy demonstrates broad potential across a range of tumor types. As such, CLN-619 has potential to treat a significant class of patients whose tumors had relapsed on or were not amenable to checkpoint inhibitor therapy.

Further data show that a CLN-619 plus pembrolizumab combination therapy was well tolerated and demonstrated meaningful clinical activity, including objective response, in tumor types typically unresponsive to pembrolizumab.

Data from CLN-619-001 (NCT05117476), a first in human dose escalation and dose expansion study of CLN-619 alone or in combination with an anti-PD1 in patients with advanced solid tumors are described in the examples. 64 patients received CLN-619 in combination with pembrolizumab (n=22) or as monotherapy (n=42).

Monotherapy efficacy has been observed across a number of tumors including confirmed responses at doses of 3 mg/kg and above. CLN-619 was tolerated at doses up to 10 mg/kg, and dose-limiting toxicities were not observed at any dose tested. In some embodiments, the CLN-619 monotherapy was administered intravenously every three weeks.

Pharmacokinetic (PK), Pharmacodynamic (PD) and mechanism of action studies were performed aimed at observing the PK and PD effects of CLN-619 in the periphery and in the tumor microenvironment.

The PK analysis for dose escalation of CLN-619 monotherapy indicates that CLN-619 half-life ranged from 61.3 to 443 hours (2.5 to 18.45 days) for dose levels ranging from 0.1 to 10 mg/kg. There was nearly a dose-proportional increase in exposure as measured by Cmax was observed between 0.1 and 10 mg/kg. Additionally, more than dose proportional increase in CLN-619 exposure as measured by AUC0-504 h was observed between 0.1 and 1 mg/kg and nearly dose-proportional increase in CLN-619 exposure was observed between 1 and 10 mg/kg dose levels.

Cytokine expression in the longitudinal serum samples from patients treated with CLN-619 monotherapy was investigated, with the data showing transient increases in cytokine levels were observed at 2-6 hrs after the first dose of CLN-619 and decreased to baseline by day 4-8. No dose-dependent increase in cytokine secretion was observed at the periphery.

There was an increase in intratumoral MICA/B expression upon treatment. Analysis of 9 paired biopsies by MultiplexIF and quantification using Image Analysis algorithm (Flagship Biosciences) showed increased MICA/B-positive staining in the majority of paired biopsies of patients on treatment (cycle 2, Day 8) as compared to baseline.

The MICA/B membrane localization in the 9 paired biopsies was also analyzed by IHC and quantified by a pathologies as a percentage of membrane staining positive cells and showed that there was increased MICA/B membrane positivity in the majority of on-treatment biopsies.

Data in the appendix shows monotherapy activity was achieved in heavily pre-treated patients, including after progression on anti-PD1 therapy. For the CLN-619 monotherapy objective responses were observed across multiple tumor types, including after progression on checkpoint inhibitor therapy and notable monotherapy activity observed in gynecological malignancies.

The data in the examples also show two case studies of subjects with endometrial tumors. In the first case study, the subject had an endometrioid endometrial cancer and showed a confirmed partial response after 3 cycles of CLN-619. The subject had had 3 prior lines of therapy and no checkpoint inhibitor intervention. The second case study subject had serous endometrial cancer and presented a confirmed partial response after 6 cycles of treatment with CLN-619. This subject had had five prior lines of therapy including dosing with pembrolizumab and levantinib. The data show that responding endometrial tumors are microsatellite-stable and have low tumor mutation burden and low neoantigen presentation index.

An analysis of the tumor microenvironment in patients with stable disease lasting greater than 6 months was performed using paired biopsies from 13 patients treated in the dose level extension cohorts at 3, 6, and 10 mg/kg dose levels. The data showed low tumor mutation burden and low neoantigen presentation in tumors with prolonged stable disease. There was increased MICA/B expression and NK-cell activation on treatment with CLN-619 in tumors from patients with stable disease lasting greater than 6 months. PDL1 expression and C8 activation increased on treatment in ⅔ paired biopsies.

The studies also show that soluble MICA (sMICA) in patient serum is a PD biomarker for CLN-619. In this study, negative serum samples spiked with recombinant sMICA or normal serum were pre-cleared of endogenous IgGs using Protein G Sepharose beads. Supernatant after bead clearance was immunoprecipitated with hIgG1 or CLN-619. sMICA remaining in the supernatant after immunoprecipitation was quantified with ELISA and plotted as a percentage of control. The studies show that CLN-619 binds to sMICA in human serum. It was further shown that sMICA levels increase in serum upon CLN-619 treatment and that sMICA levels are a surrogate for target engagement by CLN-619. This is shown by modeling of CLN-619 drug concentrations that predicted sMICA levels. Moreover, there was no sink effect observed. CLN-619 is found in approximately 1000× excess in patient blood relative to sMICA levels, suggesting that only a small fraction of CLN-619 is bound to sMICA at any time. This small fraction of CLN-619 bound to sMICA was not observed to be clinically relevant.

Thus, consistent with the proposed mechanism of action of CLN-619, there is increased membrane localization of MICA/B upon treatment with CLN-619. Therefore, the present invention shows methods of increasing MICA/B location/localization of MICA/B in a subject by administering to that subject an effective amount of CLN-619.

In certain embodiments, it is shown that presence of sMICA is a pharmacodynamic biomarker of CLN-619. The methods of the present invention include monitoring the levels of sMICA in the presence and absence of CLN-619 therapy wherein an increase in the present of sMICA is indicative that the CLN-619 is therapeutically effective. The data herein shows that the levels of increase in sMICA are dose responsive and dependent on the dose of CLN-619 administered.

Other methods described herein use sMICA as a measure of target engagement by CLN-619. These methods, for example relate to methods of determining the effectiveness or efficacy of CLN-619 in a subject comprising determining the presence of sMICA in the serum of said subject before and after administration of CLN-619, wherein an increase in the present of sMICA is indicative of said effectiveness or efficacy.

Moreover, the data showing objective responses and prolonged stable disease (6 months or greater) shown in tumors with low TMB and low antigen presentation index supports the potential for CLN-619 to elicit response independent of proficient antigen presentation machinery. This is consistent with proposed multimodal mechanisms of action for CLN-619. Thus, presented herein are methods of treating a solid tumor with CLN-619, wherein said treatment results in at least a partial response of said tumor to said CLN-619. The methods also may comprise showing a slowing or a stabilization of the disease for a period of at least six months. Thus, the methods of the present invention could also be used to prolong the life of a subject by a period of at least 6 months using the compositions of the present invention. These and other aspects of the present invention are shown in further detail herein below.

### Definitions

Unless defined otherwise, all technical and scientific terms used herein have the same meaning as commonly understood by one of ordinary skill in the art to which the disclosure pertains.

As used herein “MICA/B” refers to MICA protein, MICB protein or both MICA and MICB proteins, including their variants, isoforms, and species homologs of human MICA/B.

As used herein “antibody” refers to a glycoprotein which exhibits binding specificity to a specific antigen. An antibody often comprises a variable domain and a constant domain in each of a heavy chain and a light chain. Accordingly, most antibodies have a heavy chain variable domain (VH) and a light chain variable domain (VL) that together form the portion of the antibody that binds to the antigen. Within each variable domain are three complementarity determining regions (CDR) which form loops in the heavy chain variable domain (VH) and light chain variable domain (VL) that contact the surface of the antigen. Antibodies herein also include intact molecules as well as functional fragments thereof, which are also referred to as an “antigen binding portion” or fragments of the antibody that are capable of binding to the antigen.

As used herein “chimeric” antibodies are antibodies having a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see e.g., Morrison et al., Proc. Natl. Acad. Sci. USA 81:6851-6855 (1984)). “Humanized antibodies” herein refers to chimeric antibodies having human sequences substituted in the antibody sequence.

The terms “recipient,” “individual,” “subject,” “host,” and “patient” are used interchangeably herein and in some cases, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the compounds or agents of the present invention to prevent or delay, to alleviate, or to arrest or inhibit development of the symptoms or conditions associated with diseases (e.g., cancer). The term “therapeutic effect” refers to the reduction, elimination, or prevention of the disease, symptoms of the disease, or side effects of the disease in the subject.

A “therapeutically effective amount” in some cases means the amount that, when administered to a subject for treating a disease, is sufficient to effect treatment for that disease.

As used herein, singular forms “a,” “and,” and “the” include plural referents unless the context clearly indicates otherwise. Thus, for example, reference to “an antibody” includes a plurality of antibodies.

“About” a number, as used herein, refers to range including the number and ranging from 10% below that number to 10% above that number. “About” a range refers to 10% below the lower limit of the range, spanning to 10% above the upper limit of the range.

Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO:Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

### MICA/B

Disclosed herein, in some embodiments, are monoclonal antibodies that bind specifically to MICA/B. Further disclosed herein, in some embodiments, are monoclonal antibodies that competitively bind to MICA/B.

Major Histocompatibility Complex (MHC) class I Chain-related gene A and gene B protein (MICA/B) are glycosylated, polymorphic and membrane-anchored non-classical MHC class I proteins. MICA/B are related to MHC class I and have similar domain structure comprising three extra-cellular Ig-like domains (alpha-1, alpha-2, and alpha-3), a transmembrane domain, and a C-terminal cytoplasmic tail. MICA/B are ligands to C-type lectin-like activating receptor Natural Killer Group 2D (NKG2D) on immune effector cells, including NK, NKT and both αβ and γδ CD8+ T cells. The interaction of MICA/B and NKG2D plays a role in tumor surveillance, and immune response.

MICA/B proteins are expressed normally at low levels in normal cells but are induced to higher levels in stressed or transformed cells (e.g. cancer cells). The interaction of NKG2D-bearing immune effector cells with stressed or diseased cells expressing MICA/B ligands on the cell surface creates a cellular immune response against the stressed/diseased cell that culminates in the death of the MICA/B expressing cells. In cancer cells, the truncated MICA/B proteins (proteins that lack the transmembrane domain and cytoplasmic tail but retain the three extracellular domain comprising alpha-1, -2, and -3 domains) are frequently shed into the blood by the action of proteases and results in the down-modulation (receptor internalization) of its intended receptor, NKG2D, on effector immune cells. In some instances, MICA/B glycoproteins are produced intracellularly that are not routinely destined to become cell surface membrane-bound, but instead are incorporated within exosomes and released outside the cell where interaction with NKG2D receptors on immune cells occurs. These truncated or soluble MICA/B ligands shed from the surface of cancer cells function like decoy molecules and lead to down-modulation of the NKG2D receptor on immune effector cells such as NK, NKT, and various CD8+ T cells. In some instances, the formation of soluble MICA/B leads to the unusual situation where the effectors of the innate defense system, whose natural role is to seek and destroy transformed cells, are shut down by the immunosuppressive actions of these decoy ligand molecules, thereby enabling the cancer cells to hide from the immune system and to grow unchecked.

### Anti-MICA/B Antibodies

Provided herein are antibodies that specifically bind to MICA/B proteins. In some embodiments, anti-MICA/B antibodies comprise at least one heavy chain and anti-MICA/B antibodies comprise at least one light chain. In some embodiments, anti-MICA/B antibodies comprise at least one heavy chain comprising a heavy chain variable domain (VH) and at least one light chain comprising a light chain variable domain (VL). Each VH and VL comprises three complementarity determining regions (CDR). The amino acid sequences of the VH and VL and the CDRs determine the antigen binding specificity and antigen binding strength of the antibody. The amino acid sequences of the heavy and light chains, VH and VL and the CDRs are summarized in Table 1.

TABLE 1
Anti-MICA/B Monoclonal Antibody Sequences

| | | SEQ ID |
|---|---|---|
| | SEQUENCE | NO: |
| 3F9H1L3L | RSSKSLLHSNLNTYLY | 1 |
| Light Chain | | |
| CDR1 | | |
| 3F9H1L3L | RMSNLAS | 2 |
| Light Chain | | |
| CDR2 | | |
| 3F9H1L3L | MQHLEYPFT | 3 |
| Light Chain | | |
| CDR3 | | |
| 3F9H1L3L | GFTFSNYAMS | 4 |
| Heavy Chain | | |
| CDR1 | | |
| 3F9H1L3L | YISPGGDYIYYADSVKG | 5 |
| Heavy Chain | | |
| CDR2 | | |
| 3F9H1L3L | DRRHYGSYAMDY | 6 |
| Heavy Chain | | |
| CDR3 | | |
| 3F9H1L3L | DIVMTQSPLSLPVTPGEPASISCRSSKSLLHSNLNTYLYWFLQKP | 7 |
| Light Chain | GQSPQILIYRMSNLASGVPDRFSGSGSGTAFTLKISRVEAEDVG | |
| Variable | VYYCMQHLEYPFTFGPGTKLEIKR | |
| domain | | |
| 3F9H1L3L | QVQLVESGGGLVKPGGSLRLSCAASGFTFSNYAMSWIRQAPGK | 8 |
| Heavy Chain | GLEWVSYISPGGDYIYYADSVKGRFTISRDNAKNSLYLQMNSL | |
| Variable | RAEDTAVYYCTTDRRHYGSYAMDYWGQGTLVTVSS | |
| domain | | |
| 3F9H1L3L | DIVMTQSPLSLPVTPGEPASISCRSSKSLLHSNLNTYLYWFLQKP | 9 |
| Light Chain | GQSPQILIYRMSNLASGVPDRFSGSGSGTAFTLKISRVEAEDVG | |
| | VYYCMQHLEYPFTFGPGTKLEIKRTVAAPSVFIFPPSDEQLKSG | |
| | TASVVCLLNNFYPREAKVQWKVDNALQSGNSQESVTEQDSKD | |
| | STYSLSSTLTLSKADYEKHKVYACEVTHQGLSSPVTKSFNRGE | |
| | C* | |
| 3F9H1L3L | QVQLVESGGGLVKPGGSLRLSCAASGFTFSNYAMSWIRQAPGK | 10 |
| Heavy Chain | GLEWVSYISPGGDYIYYADSVKGRFTISRDNAKNSLYLQMNSL | |
| | RAEDTAVYYCTTDRRHYGSYAMDYWGQGTLVTVSSASTKGPS | |
| | VFPLAPSSKSTSGGTAALGCLVKDYFPEPVTVSWNSGALTSGV | |
| | HTFPAVLQSSGLYSLSSVVTVPSSSLGTQTYICNVNHKPSNTKV | |
| | DKKVEPKSCDKTHTCPPCPAPELLGGPSVFLFPPKPKDTLMISR | |
| | TPEVTCVVVDVSHEDPEVKFNWYVDGVEVHNAKTKPREEQY | |
| | NSTYRVVSVLTVLHQDWLNGKEYKCKVSNKALPAPIEKTISKA | |
| | KGQPREPQVYTLPPSREEMTKNQVSLTCLVKGFYPSDIAVEWE | |
| | SNGQPENNYKTTPPVLDSDGSFFLYSKLTVDKSRWQQGNVFSC | |
| | SVMHEALHNHYTQKSLSLSPG ** | |

In some embodiments, the antibodies specifically bind to a MICA protein. In some embodiments, the antibodies specifically bind to a MICB protein. In some embodiments, the antibodies specifically bind to both MICA and MICB protein. In some embodiments, the antibodies bind to an alpha-3 domain of a MICA protein. In some embodiments, the antibodies bind to an alpha-3 domain of a MICB protein. In some embodiments, the antibodies bind to an alpha-3 domain of both MICA and MICB protein. In some embodiments, the antibodies bind to a MICA protein that is membrane-bound MICA protein. In some embodiments, the antibodies bind to a MICA protein that is soluble MICA protein. In some embodiments, the antibodies bind to a MICA protein that is both membrane-bound MICA protein and soluble MICA protein. In some embodiments, the antibodies bind to a MICB protein that is membrane-bound MICB protein. In some embodiments, the antibodies bind to a MICB protein that is soluble MICB protein. In some embodiments, the antibodies bind to a MICB protein that is both membrane-bound MICB protein and soluble MICB protein.

In some embodiments, antibodies that specifically bind to MICA/B are monoclonal antibodies. In some embodiments, the antibody is an antigen binding fragment. In some embodiments, the antibody is selected from a whole immunoglobulin, an scFv, a Fab, a F(ab′)2, or a disulfide linked Fv. In some embodiments, the antibody is an IgG or an IgM. In some embodiments, the antibody is humanized. In some embodiments, the antibody is chimeric.

### MICA/B Antibody Complementarity Determining Regions

Disclosed herein are antibodies that specifically bind to MICA/B having a light chain comprising a light chain complementarity determining region (CDR). In some embodiments, antibodies binding to MICA/B comprise a light chain CDR sequence having an amino acid sequence at least about 70% identical to at least one of the amino acid sequences set forth as SEQ ID NOS: 1-3. In some embodiments, antibodies binding to MICA/B comprise a light chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 1-3. In some embodiments, antibodies binding to MICA/B comprise a light chain CDR sequence having an amino acid sequence 100% identical to at least one of the amino acid sequences set forth as SEQ ID NOS: 1-3.

Further disclosed herein are antibodies that specifically bind to MICA/B having a heavy chain comprising a heavy chain complementarity determining region (CDR). In some embodiments, antibodies binding to MICA/B comprise a heavy chain CDR sequence having an amino acid sequence at least about 70% identical to at least one of the amino acid sequences set forth as SEQ ID NOS: 4-6. In some embodiments, antibodies binding to MICA/B comprise a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6. In some embodiments, antibodies binding to MICA/B comprise a heavy chain CDR sequence having an amino acid sequence 100% identical to at least one of the amino acid sequences set forth as SEQ ID NOS: 4-6.

Also disclosed herein are antibodies binding to MICA/B comprising a light chain complementarity determining region (CDR) and a heavy chain complementarity determining region (CDR). In some embodiments, antibodies binding to MICA/B comprise a light chain CDR sequence having an amino acid sequence at least about 70% identical to at least one of the amino acid sequences set forth as SEQ ID NOS: 1-3 and a heavy chain CDR sequence having an amino acid sequence at least about 70% identical to at least one of the amino acid sequences set forth as SEQ ID NOS: 4-6. In some embodiments, antibodies binding to MICA/B comprise a light chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 1-3 and a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6. In some embodiments, antibodies binding to MICA/B comprise a light chain CDR sequence having an amino acid sequence 100% identical to at least one of the amino acid sequences set forth as SEQ ID NOS: 1-3 and a heavy chain CDR sequence having an amino acid sequence at least about 100% identical to at least one of the amino acid sequences set forth as SEQ ID NOS: 4-6.

In some embodiments, antibodies binding to MICA/B comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, and a light chain CDR3 having an amino acid sequence at least about 70% identical to an amino acid sequence set forth as SEQ ID NO: 3. In some embodiments, antibodies binding to MICA/B comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3. In some embodiments, antibodies binding to MICA/B comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, and a light chain CDR3 having an amino acid sequence 100% identical to an amino acid sequence set forth as SEQ ID NO: 3.

In some embodiments, antibodies binding to MICA/B comprise at least one of a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, a heavy chain CDR3 having an amino acid sequence at least about 70% identical to an amino acid sequence set forth as SEQ ID NO: 6. In some embodiments, antibodies binding to MICA/B comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, a heavy chain CDR3 having an amino acid sequence 100% identical to an amino acid sequence set forth as SEQ ID NO: 6.

In some embodiments, antibodies binding to MICA/B comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, a light chain CDR3 having an amino acid sequence at least about 70% identical to an amino acid sequence set forth as SEQ ID NO: 3, a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, and a heavy chain CDR3 having an amino acid sequence at least about 70% identical to an amino acid sequence set forth as SEQ ID NO: 6. In some embodiments, antibodies binding to MICA/B comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies binding to MICA/B comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, a light chain CDR3 having an amino acid sequence 100% identical to an amino acid sequence set forth as SEQ ID NO: 3, a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, and a heavy chain CDR3 having an amino acid sequence 100% identical to an amino acid sequence set forth as SEQ ID NO: 6.

### MICA/B Antibody Variable Domain

Disclosed herein are antibodies that specifically bind to MICA/B having a light chain comprising a light chain variable domain (VL). In some embodiments, antibodies binding to MICA/B comprise a light chain variable domain (VL) having an amino acid sequence at least about 70% identical to an amino acid sequence set forth as SEQ ID NO: 7. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7. In some embodiments, the VL has an amino acid sequence 100% identical to an amino acid sequence set forth as SEQ ID NO: 7.

Further disclosed herein are antibodies that specifically bind to MICA/B having a heavy chain comprising a heavy chain variable domain (VH). In some embodiments, antibodies binding to MICA/B comprise a heavy chain variable domain (VH) having an amino acid sequence at least about 70% identical to an amino acid sequence set forth as SEQ ID NO: 8. In some embodiments the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8. In some embodiments, the VH has an amino acid sequence 100% identical to an amino acid sequence set forth as SEQ ID NO: 8.

Also disclosed herein are antibodies binding to MICA/B comprising a light chain variable domain (VL) and a heavy chain variable domain (VH). In some embodiments, antibodies binding to MICA/B comprise a light chain variable domain (VL) having an amino acid sequence at least about 70% identical to an amino acid sequence set forth as SEQ ID NO: 7 and a heavy chain variable domain (VH) having an amino acid sequence at least about 70% identical to an amino acid sequence set forth as SEQ ID NO: 8. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8. In some embodiments, the VL has an amino acid sequence 100% identical to an amino acid sequence set forth as SEQ ID NO: 7 and the VH has an amino acid sequence 100% identical to an amino acid sequence set forth as SEQ ID NO: 8.

### MICA/B Antibody Heavy and Light Chain

Disclosed herein are antibodies that specifically bind to MICA/B having a light chain. In some embodiments, antibodies binding to MICA/B comprise a light chain having an amino acid sequence at least about 70% identical to an amino acid sequence set forth as SEQ ID NO: 9. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9. In some embodiments, the light chain has an amino acid sequence 100% identical to an amino acid sequence set forth as SEQ ID NO: 9.

Further disclosed herein are antibodies that specifically bind to MICA/B having a heavy chain. In some embodiments, antibodies binding to MICA/B comprise a heavy chain having an amino acid sequence at least about 70% identical to an amino acid sequence set forth as SEQ ID NO: 10. In some embodiments the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10. In some embodiments, the heavy chain has an amino acid sequence 100% identical to an amino acid sequence set forth as SEQ ID NO: 10.

Also disclosed herein are antibodies binding to MICA/B comprising a light chain and a heavy chain. In some embodiments, antibodies binding to MICA/B comprise a light chain having an amino acid sequence at least about 70% identical to an amino acid sequence set forth as SEQ ID NO: 9 and a heavy chain having an amino acid sequence at least about 70% identical to an amino acid sequence set forth as SEQ ID NO: 10. In some embodiments the light chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9 and the heavy chain has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10. In some embodiments, the light chain has an amino acid sequence 100% identical to an amino acid sequence set forth as SEQ ID NO: 9 and the heavy chain has an amino acid sequence 100% identical to an amino acid sequence set forth as SEQ ID NO: 10.

### Methods of Treatment and Use

Provided herein are methods of treating cancer in an individual in need thereof comprising administration of an anti-MICA/B antibody disclosed herein.

In some embodiments, the antibodies disclosed herein reduce the level of soluble MICA protein. In some embodiments, the antibodies disclosed herein reduce the level of soluble MICB protein. In some embodiments, the antibodies disclosed herein reduce the level of both soluble MICA protein and soluble MICB protein. In some embodiments, the antibodies disclosed herein reduce shedding of soluble MICA protein. In some embodiments, the antibodies disclosed herein reduce shedding of soluble MICB protein. In some embodiments, the antibodies disclosed herein reduce shedding of both soluble MICA protein and soluble MICB protein. In some embodiments, the antibodies disclosed herein inhibit shedding of soluble MICA protein. In some embodiments, the antibodies disclosed herein inhibit shedding of soluble MICB protein. In some embodiments, the antibodies disclosed herein inhibit shedding of both soluble MICA protein and soluble MICB protein. In some embodiments, treatment with the disclosed anti-MICA/B antibodies results in alleviating or inhibiting the immunosuppressive environment by preventing or blocking the interaction between soluble MICA/B and NKG2D receptors in an individual.

### Subjects

In some embodiments, subjects described herein are subjects having a cancer. The subject can be a mammal, e.g., a primate, e.g., a higher primate, e.g., a human (e.g., a patient having, or at risk of having, a disorder described herein, e.g., a cancer). In one embodiment, the subject is in need of enhancing an immune response. In one embodiment, the subject has, or is at risk of, having a disorder described herein, e.g., a cancer as described herein. In certain embodiments, the subject is, or is at risk of being, immunocompromised.

In some embodiments, the subject to be treated is one who has previously been treated with a checkpoint inhibitor therapy. In some embodiments, the checkpoint inhibitor therapy comprises a PD-1 inhibitor (e.g., an anti-PD-1 antibody). In some embodiments, the subject responded (e.g., had a complete response or partial response) to the PD-1 inhibitor. In some embodiments, the subject had a period of response of 1, 2, 3, 4, 5, 6, 7, 8, 9, 10, 11, 12, 18, 24, 30, 36, 42, 48, 54, 60 months or more following administration of the PD-1 inhibitor. In some embodiments, the subject progressed following a period of response to the PD-1 inhibitor. In some embodiments, the subject receives treatment with the anti-MICA/B antibody following progression after the period of response to the PD-1 inhibitor.

In some embodiments, the subject had stable disease upon administration of the PD-1 inhibitor. In some embodiments, the subject had a period of stable disease of 1, 2, 3, 4, 5, 6, 7, 8, 9, 10, 11, 12, 18, 24, 30, 36, 42, 48, 54, 60 months or more. In some embodiments, the subject progressed following a period of stable disease after treatment with the PD-1 inhibitor. In some embodiments, the subject receives treatment with the anti-MICA/B antibody following progression after the period of stable disease after treatment with the PD-1 inhibitor.

In some embodiments, treatment with the PD-1 inhibitor ceased before initiation of administration of the anti-MICA/B antibody. In some embodiments, treatment with the PD-1 inhibitor continues after initiation of administration of the anti-MICA/B antibody.

In some embodiments, treatment with the anti-MICA/B antibody results in a response (e.g., a complete response or partial response) or stable disease. In some embodiments, the response after administration of the anti-MICA/B antibody persists for at least 1, 2, 3, 4, 5, 6, 7, 8, 9, 10, 11, 12, 18, 24, 30, 36, 42, 48, 54, 60 months or more.

In some embodiments, the PD-1 inhibitor comprises pimivalimab, pembrolizumab, nivolumab, cemiplimab, AMP-224, APM-514, or spartalizumab.

In some embodiments, the subject who has previously been treated with a PD-1 inhibitor has also previously been treated with one or more of a VEGF inhibitor, an EGFR inhibitor, and/or a hormone therapy.

In some embodiments, the subject to be treated is one who has previously been treated with a VEGF inhibitor. In some embodiments, the subject responded (e.g., had a complete response or partial response) to the VEGF inhibitor. In some embodiments, the subject had a period of response of 1, 2, 3, 4, 5, 6, 7, 8, 9, 10, 11, 12, 18, 24, 30, 36, 42, 48, 54, 60 months or more following administration of the VEGF inhibitor. In some embodiments, the subject progressed following a period of response to the VEGF inhibitor. In some embodiments, the subject receives treatment with the anti-MICA/B antibody following progression after the period of response to the VEGF inhibitor.

In some embodiments, the subject had stable disease upon administration of the VEGF inhibitor. In some embodiments, the subject had a period of stable disease of 1, 2, 3, 4, 5, 6, 7, 8, 9, 10, 11, 12, 18, 24, 30, 36, 42, 48, 54, 60 months or more. In some embodiments, the subject progressed following a period of stable disease after treatment with the VEGF inhibitor. In some embodiments, the subject receives treatment with the anti-MICA/B antibody following progression after the period of stable disease after treatment with the VEGF inhibitor.

In some embodiments, treatment with the VEGF inhibitor ceased before initiation of administration of the anti-MICA/B antibody. In some embodiments, treatment with the VEGF inhibitor continues after initiation of administration of the anti-MICA/B antibody.

In some embodiments, treatment with the anti-MICA/B antibody results in a response (e.g., a complete response or partial response) or stable disease. In some embodiments, the response after administration of the anti-MICA/B antibody persists for at least 1, 2, 3, 4, 5, 6, 7, 8, 9, 10, 11, 12, 18, 24, 30, 36, 42, 48, 54, 60 months or more.

In some embodiments, the VEGF inhibitor comprises a small molecule tyrosine kinase inhibitor (TKI) (e.g., sunitinib, sorafenib, axitinib, pazopanib or lenvatinib) or an anti-VEGF antibody (e.g., bevacizumab or ranibizumab).

In some embodiments, the subject who has previously been treated with a VEGF inhibitor has also previously been treated with one or more of a PD-1 inhibitor, an EGFR inhibitor, and/or a hormone therapy.

In some embodiments, the subject to be treated is one who has previously been treated with an EGFR inhibitor. In some embodiments, the subject responded (e.g., had a complete response or partial response) to the EGFR inhibitor. In some embodiments, the subject had a period of response of 1, 2, 3, 4, 5, 6, 7, 8, 9, 10, 11, 12, 18, 24, 30, 36, 42, 48, 54, 60 months or more following administration of the EGFR inhibitor. In some embodiments, the subject progressed following a period of response to the EGFR inhibitor. In some embodiments, the subject receives treatment with the anti-MICA/B antibody following progression after the period of response to the EGFR inhibitor.

In some embodiments, the subject had stable disease upon administration of the EGFR inhibitor. In some embodiments, the subject had a period of stable disease of 1, 2, 3, 4, 5, 6, 7, 8, 9, 10, 11, 12, 18, 24, 30, 36, 42, 48, 54, 60 months or more. In some embodiments, the subject progressed following a period of stable disease after treatment with the EGFR inhibitor. In some embodiments, the subject receives treatment with the anti-MICA/B antibody following progression after the period of stable disease after treatment with the EGFR inhibitor.

In some embodiments, treatment with the EGFR inhibitor ceased before initiation of administration of the anti-MICA/B antibody. In some embodiments, treatment with the EGFR inhibitor continues after initiation of administration of the anti-MICA/B antibody.

In some embodiments, treatment with the anti-MICA/B antibody results in a response (e.g., a complete response or partial response) or stable disease. In some embodiments, the response after administration of the anti-MICA/B antibody persists for at least 1, 2, 3, 4, 5, 6, 7, 8, 9, 10, 11, 12, 18, 24, 30, 36, 42, 48, 54, 60 months or more.

In some embodiments, the EGFR inhibitor comprises a small molecule tyrosine kinase inhibitor (TKI) (e.g., gefitinib, erlotinib, afatinib, dacomitinib, or osimertinib) or an anti-EGFR antibody, e.g., cetuximab, panitumumab, or necitumumab).

In some embodiments, the subject who has previously been treated with an EGFR inhibitor has also previously been treated with one or more of a PD-1 inhibitor, an VEGF inhibitor, and/or a hormone therapy.

In some embodiments, the subject to be treated is one who has previously been treated with a hormone therapy. In some embodiments, the subject responded (e.g., had a complete response or partial response) to the hormone therapy. In some embodiments, the subject had a period of response of 1, 2, 3, 4, 5, 6, 7, 8, 9, 10, 11, 12, 18, 24, 30, 36, 42, 48, 54, 60 months or more following administration of the hormone therapy. In some embodiments, the subject progressed following a period of response to the hormone therapy. In some embodiments, the subject receives treatment with the anti-MICA/B antibody following progression after the period of response to the hormone therapy.

In some embodiments, the subject had stable disease upon administration of the hormone therapy. In some embodiments, the subject had a period of stable disease of 1, 2, 3, 4, 5, 6, 7, 8, 9, 10, 11, 12, 18, 24, 30, 36, 42, 48, 54, 60 months or more. In some embodiments, the subject progressed following a period of stable disease after treatment with the hormone therapy. In some embodiments, the subject receives treatment with the anti-MICA/B antibody following progression after the period of stable disease after treatment with the hormone therapy.

In some embodiments, treatment with the hormone therapy ceased before initiation of administration of the anti-MICA/B antibody. In some embodiments, treatment with the hormone therapy continues after initiation of administration of the anti-MICA/B antibody.

In some embodiments, treatment with the anti-MICA/B antibody results in a response (e.g., a complete response or partial response) or stable disease. In some embodiments, the response after administration of the anti-MICA/B antibody persists for at least 1, 2, 3, 4, 5, 6, 7, 8, 9, 10, 11, 12, 18, 24, 30, 36, 42, 48, 54, 60 months or more.

In some embodiments, the hormone therapy comprises an aromatase inhibitor (AI) (e.g., anastrozole), a selective estrogen receptor modulator (SERM), a luteinizing hormone-releasing hormone (LHRH) agonist, an anti-androgen, a CYP17 inhibitor, a progestin, an adrenolytic, or an estrogen receptor antagonist.

In some embodiments, the subject who has previously been treated with a hormone therapy has also previously been treated with one or more of a PD-1 inhibitor, an VEGF inhibitor, and/or an EGFR inhibitor.

In some embodiments, the subject to be treated is one who has an endometrial cancer. In some embodiments, the subject has previously been treated with a PD-1 inhibitor (e.g., an anti-PD-1 antibody). In some embodiments, the subject has previously been treated with a VEGF inhibitor or hormonal therapy. In some embodiments, the subject has not previously been treated with a PD-1 inhibitor (e.g., an anti-PD-1 antibody). In some embodiments, the endometrial cancer is HER2+. In some embodiments, the endometrial cancer is one or both of ER+ or PR+. In some embodiments, the cancer is mutant for one or more of: EGFR, TP53, HER2, PTEN, ARIDIA, CTNNB1, or PLK3R1.

In some embodiments, the subject to be treated is one who has a parotid cancer. In some embodiments, the parotid cancer overexpresses one or both of EGFR and HER2. In some embodiments, parotid cancer is a mucoepidermoid parotid cancer. In some embodiments, the cancer is mutant for one or more of: EGFR, TP53, HER2, PTEN, ARIDIA, CTNNB1, or PLK3R1.

### Cancers

In certain embodiments, the cancer treated according to the methods described herein, includes but is not limited to, a tumor of the female genital tract, a salivary gland tumor, a breast cancer, a prostate cancer, a lung cancer, a colon cancer, a sarcoma, a melanoma, a rectal cancer, a thyroid cancer, a kidney cancer, a gastrointestinal cancer, a peritoneal cancer, a pancreatic cancer, or a metastatic lesion thereof. In some embodiments, the tumor is an advanced solid tumor. In some embodiments, the tumor is a head and neck tumor, e.g., a parotid tumor.

In some embodiments, the compositions, including, but not limited to, pharmaceutical compositions including anti-MICA/B antibodies, are particularly useful for the treatment of high (large) tumor burden. Tumor burden refers to the number of cancer cells, the size of a tumor, or the amount of cancer in the body. High tumor burden may be used as a clinical biomarker with negative prognostic value in several solid tumors.

In some embodiments, the pharmaceutical compositions including anti-MICA/B antibodies are particularly useful in the treatment of tumors of the female genital. In some embodiments, the tumor of the female genital tract is an endometrial tumor, an ovarian cancer, or a cervical cancer. In some embodiments, the salivary gland tumor is a mucoepidermoid tumor such as a parotid tumor or an adenoid cystic sarcoma. In some embodiments, the cancer is a virally induced cancer. Virally induced cancers include those associated with infection of human papilloma virus (HPV), Epstein-Barr virus (EBV), or cytomegalovirus (CMV). In some embodiments, the sarcoma is a mediastinal intimal sarcoma or a leiomyosarcoma. In some embodiments, the peritoneal cancer is a peritoneal mesothelioma. In some embodiments, the rectal cancer is a squamous cell carcinoma of the rectum. In some embodiments, the lung cancer is a non-small cell lung cancer (NSCLC) or a mesothelioma. In some embodiments, the gastrointestinal cancer is a cancer of the duodenum. In some embodiments, the colorectal cancer is a caecal cancer.

In some embodiments, the thyroid cancer is a thyroid carcinoma. In some embodiments, the cancer is a bladder urothelial carcinoma. In some embodiments, the cancer is an adrenocortical carcinoma. In some embodiments, the cancer is a head and neck squamous cell carcinoma. In some embodiments, the cervical cancer is a cervical squamous cell carcinoma or an endocervical carcinoma. In some embodiments, the cancer is an esophageal carcinoma. In some embodiments, the cancer is a lung squamous cell carcinoma. In some embodiments, the cancer is a lymphoid neoplasm diffuse large B-cell lymphoma. In some embodiments, the cancer is a thymoma. In some embodiments, the cancer is an acute myeloid leukemia (AML). In some embodiments, the cancer is a uveal melanoma. In some embodiments, the cancer is a prostate adenocarcinoma. In some embodiments, the cancer is a kidney chromophobe. In some embodiments, the cancer is a brain lower grade glioma. In some embodiments, the cancer is a liver hepatocellular carcinoma. In some embodiments, the cancer is a skin cutaneous melanoma. In some embodiments, the cancer is a pheochromocytoma or a paraganglioma. In some embodiments, the cancer is a uterine carcinosarcoma. In some embodiments, the cancer is an ovarian serous cystadenocarcinoma. In some embodiments, the cancer is a kidney renal papillary carcinoma. In some embodiments, the cancer is a glioblastoma multiforme. In some embodiments, the cancer is a cholangiocarcinoma. In some embodiments, the cancer is a testicular germ cell tumor. In some embodiments, the cancer is a breast invasive carcinoma. In some embodiments, the cancer is a kidney renal clear cell carcinoma. In some embodiments, the cancer is a lung adenocarcinoma. In some embodiments, the cancer is a stomach adenocarcinoma. In some embodiments, the cancer is a pancreatic adenocarcinoma. In some embodiments, the cancer is a uterine corpus endometrial carcinoma. In some embodiments, the cancer is a rectal adenocarcinoma. In some embodiments, the cancer is a colon adenocarcinoma.

In some embodiments, the cancer has metastasized. In some embodiments, the cancer is mutant for one or more of: EGFR, TP53, HER2, PTEN, ARIDIA, CTNNB1, or PLK3R1.

In some embodiments, the cancer is characterized by a low level of PD-L1 expression. In some embodiments, the cancer is characterized by a high level of PD-L1 expression. In some embodiments, the cancer is characterized by a low tumor mutation burden (TMB). In some embodiments, the cancer is characterized by a high tumor mutation burden (TMB). In some embodiments, the cancer is an immunologically cold cancer. In some embodiments, the cancer is an immunologically hot cancer. In some embodiments, the cancer is a hormone-sensitive cancer. In some embodiments, the cancer is characterized by an overexpression of oncogenic drivers. In some embodiments, the cancer expresses one or more of: EGFR, ER, PR, or HER2.

### Dosages and Dosage Regimens

Therapeutically effective amounts or dosages are contemplated to include dosages of about 0.01 mg/kg to about 20 mg/kg, about for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5 mg/kg, about 5.1 mg/kg, about 5.2 mg/kg, about 5.3 mg/kg, about 5.4 mg/kg, about 5.5 mg/kg, about 5.6 mg/kg, about 5.7 mg/kg, about 5.8 mg/kg, about 5.9 mg/kg, about 6 mg/kg, about 6.1 mg/kg, about 6.2 mg/kg, about 6.3 mg/kg, about 6.4 mg/kg, about 6.5 mg/kg, about 6.6 mg/kg, about 6.7 mg/kg, about 6.8 mg/kg, about 6.9 mg/kg, about 7 mg/kg, about 7.1 mg/kg, about 7.2 mg/kg, about 7.3 mg/kg, about 7.4 mg/kg, about 7.5 mg/kg, about 7.6 mg/kg, about 7.7 mg/kg, about 7.8 mg/kg, about 7.9 mg/kg, about 8 mg/kg, about 8.1 mg/kg, about 8.2 mg/kg, about 8.3 mg/kg, about 8.4 mg/kg, about 8.5 mg/kg, about 8.6 mg/kg, about 8.7 mg/kg, about 8.8 mg/kg, about 8.9 mg/kg, about 9 mg/kg, about 9.1 mg/kg, about 9.2 mg/kg, about 9.3 mg/kg, about 9.4 mg/kg, about 9.5 mg/kg, about 9.6 mg/kg, about 9.7 mg/kg, about 9.8 mg/kg, about 9.9 mg/kg, about 10 mg/kg, about 10.1 mg/kg, about 10.2 mg/kg, about 10.3 mg/kg, about 10.4 mg/kg, about 10.5 mg/kg, about 10.6 mg/kg, about 10.7 mg/kg, about 10.8 mg/kg, about 10.9 mg/kg, about 11 mg/kg, about 11.1 mg/kg, about 11.2 mg/kg, about 11.3 mg/kg, about 11.4 mg/kg, about 11.5 mg/kg, about 11.6 mg/kg, about 11.7 mg/kg, about 11.8 mg/kg, about 11.9 mg/kg, about 12 mg/kg, about 12.1 mg/kg, about 12.2 mg/kg, about 12.3 mg/kg, about 12.4 mg/kg, about 12.5 mg/kg, about 12.6 mg/kg, about 12.7 mg/kg, about 12.8 mg/kg, about 12.9 mg/kg, about 13 mg/kg, about 13.1 mg/kg, about 13.2 mg/kg, about 13.3 mg/kg, about 13.4 mg/kg, about 13.5 mg/kg, about 13.6 mg/kg, about 13.7 mg/kg, about 13.8 mg/kg, about 13.9 mg/kg, about 14 mg/kg, about 14.1 mg/kg, about 14.2 mg/kg, about 14.3 mg/kg, about 14.4 mg/kg, about 14.5 mg/kg, about 14.6 mg/kg, about 14.7 mg/kg, about 14.8 mg/kg, about 14.9 mg/kg, about 15 mg/kg, about 15.1 mg/kg, about 15.2 mg/kg, about 15.3 mg/kg, about 15.4 mg/kg, about 15.5 mg/kg, about 15.6 mg/kg, about 15.7 mg/kg, about 15.8 mg/kg, about 15.9 mg/kg, about 16 mg/kg, about 16.1 mg/kg, about 16.2 mg/kg, about 16.3 mg/kg, about 16.4 mg/kg, about 16.5 mg/kg, about 16.6 mg/kg, about 16.7 mg/kg, about 16.8 mg/kg, about 16.9 mg/kg, about 17 mg/kg, about 17.1 mg/kg, about 17.2 mg/kg, about 17.3 mg/kg, about 17.4 mg/kg, about 17.5 mg/kg, about 17.6 mg/kg, about 17.7 mg/kg, about 17.8 mg/kg, about 17.9 mg/kg, about 18 mg/kg, about 18.1 mg/kg, about 18.2 mg/kg, about 18.3 mg/kg, about 18.4 mg/kg, about 18.5 mg/kg, about 18.6 mg/kg, about 18.7 mg/kg, about 18.8 mg/kg, about 18.9 mg/kg, about 19 mg/kg, about 19.1 mg/kg, about 19.2 mg/kg, about 19.3 mg/kg, about 19.4 mg/kg, about 19.5 mg/kg, about 19.6 mg/kg, about 19.7 mg/kg, about 19.8 mg/kg, about 19.9 mg/kg, about or 20 mg/kg.

In some embodiments, the anti-MICA/B antibody is administered at a dose of between about 0.1 mg/kg to about 10 mg/kg. In some embodiments, the anti-MICA/B antibody is administered at a dose of between about 3 mg/kg to about 10 mg/kg. In some embodiments, the anti-MICA/B antibody is administered at a dose of about 0.1 mg/kg. In some embodiments, the anti-MICA/B antibody is administered at a dose of about 0.3 mg/kg. In some embodiments, the anti-MICA/B antibody is administered at a dose of about 1 mg/kg. In some embodiments, the anti-MICA/B antibody is administered at a dose of about 3 mg/kg. In some embodiments, the anti-MICA/B antibody is administered at a dose of about 6 mg/kg. In some embodiments, the anti-MICA/B antibody is administered at a dose of about 10 mg/kg.

Methods of treatment herein comprise one or more administrations of anti-MICA/B antibodies in doses disclosed herein. In some embodiments, methods comprise one administration of anti-MICA/B antibodies. In some embodiments, methods comprise two administrations of anti-MICA/B antibodies. In some embodiments, methods comprise three administrations of anti-MICA/B antibodies. In some embodiments, methods comprise four administrations of anti-MICA/B antibodies. In some embodiments, methods comprise five administrations of anti-MICA/B antibodies. In some embodiments, methods comprise six administrations of anti-MICA/B antibodies. In some embodiments, methods comprise more than six administrations of anti-MICA/B antibodies.

In some embodiments, the anti-MICA/B antibodies are administered according to a dosing interval (e.g., a cycle). In some embodiments, one or more administrations of anti-MICA/B antibodies are administered daily. In some embodiments, one or more administrations of anti-MICA/B antibodies are administered weekly. In some embodiments, one or more administrations of anti-MICA/B antibodies are administered biweekly (Q2W). In some embodiments, one or more administrations of anti-MICA/B antibodies are administered every three weeks (Q3W). In some embodiments, one or more administrations of anti-MICA/B antibodies are administered monthly. In some embodiments, one or more administrations of anti-MICA/B antibodies are administered every three months. In some embodiments, one or more administrations of anti-MICA/B antibodies are administered every six months. In some embodiments, one or more administrations of anti-MICA/B antibodies are administered yearly.

In some embodiments, the anti-MICA/B antibodies are administered according to a dosing interval (e.g., a cycle). In some embodiments, the dosing interval comprises a three-week cycle and the anti-MICA/B antibody is administered once every three weeks (Q3W). In some embodiments, the dosing interval is repeated at least 1 time, at least 2 times, at least 3 times, at least 4 times, at least 5 times, at least 6 times, at least 7 times, at least 8 times, at least 9 times, or at least 10 times. In some embodiments, he repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, or 5 years.

In some embodiments, prior to administration of an anti-MICA/B antibody, the subject receives one or more agents as a premedication.

In some embodiments, the premedication comprises acetaminophen or ibuprofen. In some embodiments, the subject receives acetaminophen or ibuprofen 30-60 minutes prior to administration of the anti-MICA/B antibody. In some embodiments, the premedication is administered orally.

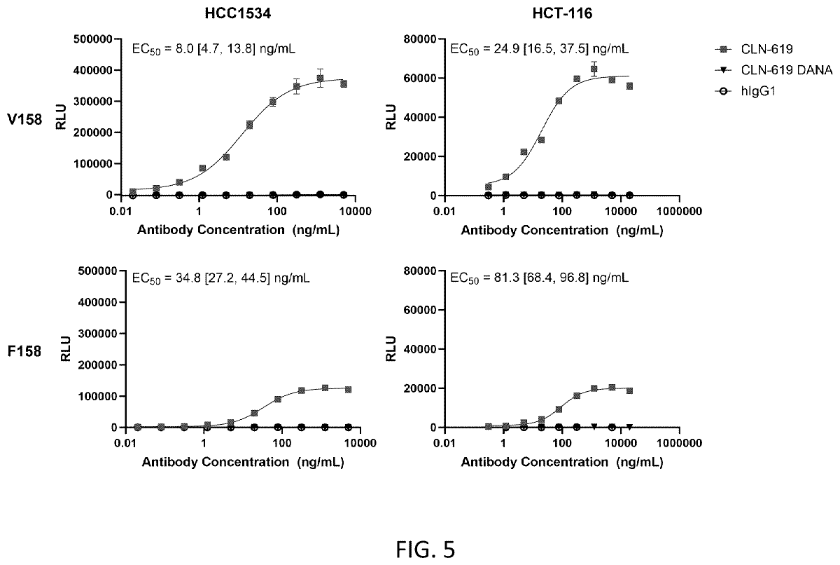

In some embodiments, the premedication comprises a corticosteroid. In some embodiments, the subject receives the corticosteroid 30-60 minutes prior to administration of the anti-MICA/B antibody. In some embodiments, corticosteroid is administered prior to the first dose of the anti-MICA/B antibody but is not administered prior to the one or more subsequent doses of the anti-MICA/B antibody. In some embodiments, the corticosteroid is administered orally or intravenously. In some embodiments, the corticosteroid is administered at a dose of between about 2 mg to about 50 mg, or indeed any standard of care corticosteroid dose that is being used in cancer therapy. In some embodiments, the corticosteroid comprises dexamethasone. As shown in Example 1 and FIG. 5 herein, an increase in patient cytokine levels was observed following the first dose of an anti-MICA/B antibody, but subsequent doses of the anti-MICA/B antibody resulted in a smaller increase or no increase. Without wishing to be bound by theory, corticosteroid administration before the first administration of the anti-MICA/B antibody can dampen a patient's immune reaction to administration of the antibody.

Any suitable route of administration is contemplated for use with the methods disclosed herein. In some embodiments, the antibody is administered by intravenous administration. In some embodiments, the antibody is administered by subcutaneous administration. In some embodiments, the antibody is administered locally. In some embodiments, the antibody is administered systemically (e.g., intravenously, intramuscularly, subcutaneously, intradermally, orally, intranasally, or sublingually). In some embodiments, the antibody is formulated as a salve, lotion, or emulsion. In some embodiments, the antibody is formulated as a solution. In some embodiments, the antibody is formulated for topical, oral, buccal, or nasal administration.

In some embodiments, the anti-MICA/B antibody is administered intravenously for about 1 hour.

In some embodiments, the individual is monitored prior to administration of the antibody. Symptoms are identified and their severity is assessed. An antibody as described herein may be administered alone or in combination with additional treatments, singly or multiply over time as discussed herein or known to one of skill in the art. In some embodiments, the individual is monitored such that the efficacy of the treatment regimen is determined. In some embodiments, a treatment regimen is modified in response to preliminary treatment outcomes, such that treatment dose or frequency or dose and frequency is altered so as to attain a desired level of subject response in light of symptom alleviation, side effect reduction, or a combination of symptom alleviation and side effect reduction.

In some embodiments, the methods of treatment disclosed herein, is a monotherapy. In some embodiments, the methods of treatment disclosed herein, is a combination therapy. In some embodiments, combination therapy comprises administrations of anti-MICA/B antibodies in combination with another therapeutic agent. In this regard, the full killing potential of NK cells in the clinical setting dealing with the immunosuppressive TME may require more than one activating stimulus, for example simultaneous engagement of both CD16A and NKG2D. The ultimate therapeutic potential for NK cell-activating therapeutics in the clinic may be enabled when delivered in combination with therapies that enhance or complement NK cell activation, such as lenalidomide, cytokines, or checkpoint inhibitors. Combination with checkpoint inhibitors provides the opportunity for dual activation of both the innate and adaptive immune response and may attenuate exhaustion of both T cells and NK cells expressing PD-1. Thus, the present disclosure contemplates 53mmune-oncology therapies in which the anti-MICA/B antibodies may be administered in combination with certain inhibitors of the immune checkpoint molecules programmed cell death 1 receptor (PD-1), T cell immunoreceptor with Ig and ITIM domains (TIGIT), T cell immunoglobulin and mucin domain-containing protein 3 (TIM-3), cluster of differentiation 96 (CD96), sialic acid binding Ig like lectin 7 (siglec-7), leukocyte-associated immunoglobulin-like receptor 1 (LAIR-1), and inhibitor receptor protein (Irp60). PD-1, considered the prototypic immune checkpoint molecule, negatively regulates T cell function upon interaction with programmed death-ligand 1 and 2 (PD-L1 and PD-L2), on cancer cells and on immune cells in the tumor microenvironment (TME) and this axis has been targeted with great success in the clinic with antagonist mAb therapies. The checkpoint inhibitors may be administered, before, after, or concurrently with the anti-MICA/B therapies of the present disclosure. Tumor-experienced NK cells have also been shown to express PD-1 and PD-L1 in both preclinical and clinical settings thereby making the use of PD-1 inhibition in combination with the MICA/B antibodies a particular embodiment contemplated herein.

In certain embodiments said composition of this disclosure comprising an anti-MICA/B antibody is in a pharmaceutical combination or therapy with at least one additional therapeutic agent selected from the group consisting of an antibody, an antibody fragment, an antibody conjugate, a cytotoxic agent, a toxin, a radionuclide, an immunomodulator, a photoactive therapeutic agent, a radiosensitizing agent, a hormone, an anti-angiogenesis agent, and combinations thereof.

In some embodiments, the therapeutic agent comprises a chemotherapeutic agent. In some embodiments, the chemotherapeutic agent include, but is not limited to, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, kinase inhibitors and immune checkpoint inhibitors.

In some embodiments, the anti-MICA/B antibodies disclosed herein are administered in combination with a therapeutic agent that engage CD16A. Those skilled in the art are aware of many chimeric, human, and humanized IgG1 antibody therapeutics that engage CD16A, including agents such as, but not limited to rituximab (targeting CD20), daratumumab (CD38), and trastuzumab (human epidermal growth factor receptor 2-HER2).

In some embodiments, the anti-MICA/B antibodies disclosed herein are administered in combination with a therapeutic agent that induces an immune response. In some embodiments, the anti-MICA/B antibodies disclosed herein are administered in combination with a therapeutic agent that inhibits downregulation of an immune response. In some embodiments, inducing an immune response comprises activation or upregulating activity of NK cells. In some embodiments, inducing an immune response comprises activation or upregulating activity of T cells. In some embodiments, the immune check point inhibitor target comprises PD-1. In some embodiments, the immune check point inhibitor target comprises PD-L1.

Specific non-limiting examples of the immune checkpoint inhibitors that target the PD-1/PD-L1 pathway include but are not limited to pimivalimab, pembrolizumab (Keytruda, MK-3475, lambrolizumab), nivolumab (e.g., (Opdivo, BMS-936558, MDX1106), cemiplimab, spartalizumab (PDR001), STI-A1110, AMP-224, AMP-514 (MEDI0680), JS001, atezolizumab (e.g., Tecentriq, MPDL3280A), avelumab (MSB0010718C), durvalumab ((MEDI4736), BMS-936559, pidilizumab (CT-011), BMS-936559 (MDX1105), and LY3300054, and CK-301. In some embodiments, the PD-1 inhibitor comprises pembrolizumab. In other embodiments, the PD-1 inhibitor comprises nivolumab.

In some embodiments, pembrolizumab comprises
a heavy chain comprising an amino
acid sequence of:
(SEQ ID NO: 11)
QVQLVQSGVEVKKPGASVKVSCKASGYTFTNYYMYWVRQAPGQGL
EWMGGINPSNGGTNFNEKFKNRVTLTTDSSTTTAYMELKSLQFDD
TAVYYCARRDYRFDMGFDYWGQGTTVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTKVDKRVESKYGPP
CPPCPAPEFLGGPSVFLFPPKPKDTLMISRTPEVTCVVVDVSQED
PEVQFNWYVDGVEVHNAKTKPREEQFNSTYRVVSVLTVLHQDWLN
GKEYKCKVSNKGLPSSIEKTISKAKGQPREPQVYTLPPSQEEMTK
NQVSLTCLVKGFYPSDIAVEWESNGQPENNYKTTPPVLDSDGSFF
LYSRLTVDKSRWQEGNVFSCSVMHEALHNHYTQKSLSLSLGK
and a light chain comprising an amino acid
sequence of:
(SEQ ID NO: 12)
EIVLTQSPATLSLSPGERATLSCRASKGVSTSGYSYLHWYQQKPG
QAPRLLIYLASYLESGVPARFSGSGSGTDFTLTISSLEPEDFAVY
YCQHSRDLPLTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC

In the following discussion pembrolizumab is used as an exemplary PD-1 inhibitor. It should be understood that instead of pembrolizumab, an alternative PD-1 inhibitor, or indeed a PD-L1 inhibitor may be used. In some embodiments, pembrolizumab is administered to the subject at a dose of about 200 mg intravenously. In some embodiments, pembrolizumab is administered to the subject once every three weeks (Q3W). In some embodiments, the subject receiving the combination therapy comprising the immune checkpoint inhibitor (e.g., pembrolizumab) has ovarian cancer, prostate cancer, rectal cancer, NSCLC, squamous cell skin cancer, or cervical cancer. In some embodiments, the anti-MICA/B antibodies described herein are administered in combination with a VEGF inhibitor. In some embodiments, the VEGF inhibitor comprises a small molecule tyrosine kinase inhibitor (TKI) (e.g., sunitinib, sorafenib, axitinib, pazopanib, or lenvatinib) or an anti-VEGF antibody (e.g., bevacizumab or ranibizumab). In some embodiments, the anti-MICA/B antibodies described herein are administered in combination with an EGFR inhibitor. In some embodiments, the EGFR inhibitor comprises a small molecule tyrosine kinase inhibitor (TKI) (e.g., gefitinib, erlotinib, afatinib, dacomitinib, or osimertinib) or an anti-EGFR antibody, e.g., cetuximab, panitumumab, or necitumumab). In some embodiments, the anti-MICA/B antibodies described herein are administered in combination with a hormone therapy. In some embodiments, the hormone therapy comprises an aromatase inhibitor (AI) (e.g., anastrozole), a selective estrogen receptor modulator (SERM), a luteinizing hormone-releasing hormone (LHRH) agonist, an anti-androgen, a CYP17 inhibitor, a progestin, an adrenolytic, or an estrogen receptor antagonist. In certain embodiments, the anti-MICA/B antibodies described herein are administered in combination with radiation therapy. In certain embodiments, the anti-MICA/B antibodies described herein are administered in combination with one or more cell therapies.

Exemplary chemical structures of certain therapeutic agents described herein are shown in Table 2.

TABLE 2
Exemplary chemical structures of therapeutic agents

| Name | Structure |
|---|---|
| Dexamethasone | 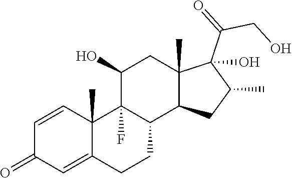 |
| Lenvatinib | 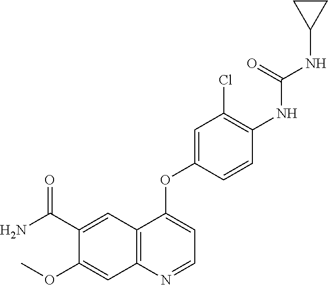 [SMILES: COc1cc2nccc(Oc3ccc(NC(=O)NC4CC4)c(Cl)c3)c2cc1C(N)=O] |
| Sunitinib | 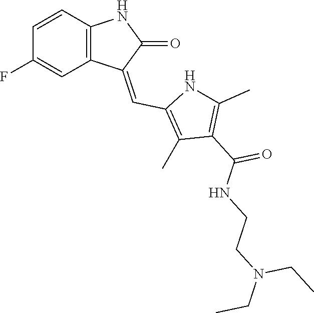 [SMILES: CCN(CC)CCNC(=O)c1c(C)[nH]c(/C=C2\C(=O)Nc3ccc(F)cc32)c1C] |
| Sorafenib | 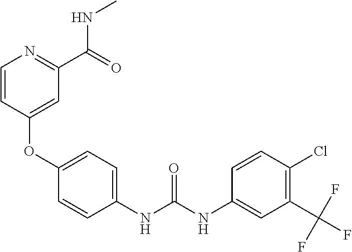 [SMILES: CNC(=O)c1cc(Oc2ccc(NC(=O)Nc3ccc(Cl)c(C(F)(F)F)c3)cc2)ccn1] |
| Axitinib | 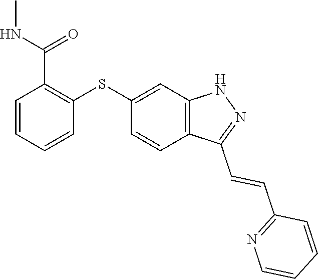 [SMILES: CNC(=O)c1ccccc1Sc1ccc2c(/C=C/c3ccccn3)n[nH]c2c1] |
| Pazopanib | 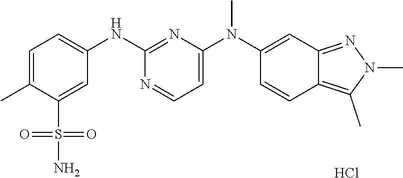 |
| Gefitinib | 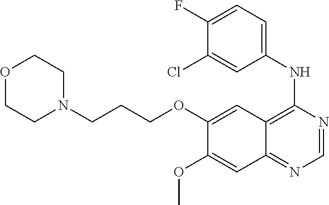 [SMILES: COc1cc2ncnc(Nc3ccc(F)c(Cl)c3)c2cc1OCCCN1CCOCC1] |
| Elotinib | 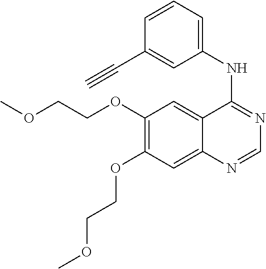 [SMILES: C#Cc1cccc(Nc2ncnc3cc(OCCOC)c(OCCOC)cc23)c1] |
| Afatinib | 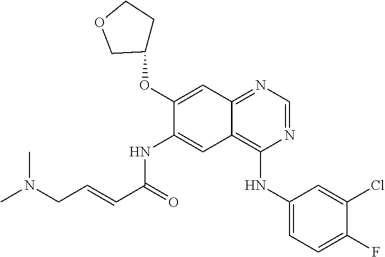 [SMILES: CN(C)C/C=C/C(=O)Nc1cc2c(Nc3ccc(F)c(Cl)c3)ncnc2cc1O[C@H]1CCOC1] |
| Dacomitinib | 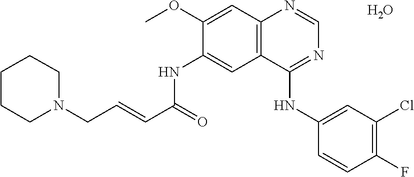 [SMILES: COc1cc2ncnc(Nc3ccc(F)c(Cl)c3)c2cc1NC(=O)/C=C/CN1CCCCC1 ; O] |
| Osimertinib | 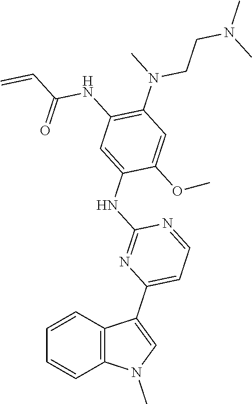 [SMILES: C=CC(=O)Nc1cc(Nc2nccc(-c3cn(C)c4ccccc34)n2)c(OC)cc1N(C)CCN(C)C] |
| Anastrozole | 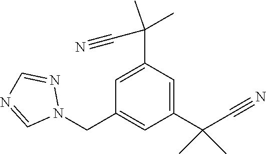 [SMILES: CC(C)(C#N)c1cc(Cn2cncn2)cc(C(C)(C)C#N)c1] |

### Pharmaceutical Compositions

Also disclosed herein are pharmaceutical compositions comprising anti-MICA/B antibodies disclosed herein and a pharmaceutically acceptable carrier or excipient.

In some embodiments, excipients for use with the compositions disclosed herein include maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In some embodiments, the compositions further comprise an additional therapeutic agent. In some embodiments, the therapeutic agent is a chemotherapeutic agent. The chemotherapeutic agents can include, among others, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In some embodiments, the antibody and the therapeutic agent are in the same formulation. In some embodiments, the antibody and the therapeutic agent are in different formulation. In some embodiments, antibody described herein is used prior to the administration of the other therapeutic agent. In some embodiments, antibody described herein is used concurrently with the administration of the other therapeutic agent. In some embodiments, antibody described herein is used subsequent to the administration of the other therapeutic agent.

Pharmaceutical formulations, in some embodiments, are made to be compatible with a particular local, regional, or systemic administration or delivery route. Thus, pharmaceutical formulations include carriers, diluents, or excipients suitable for administration by particular routes. Specific non-limiting examples of routes of administration for compositions herein are parenteral, e.g., intravenous, intra-arterial, intradermal, intramuscular, subcutaneous, intra-pleural, transdermal (topical), transmucosal, intra-cranial, intra-spinal, intra-ocular, rectal, oral (alimentary), mucosal administration, and any other formulation suitable for the treatment method or administration protocol.

In some embodiments, solutions or suspensions used for parenteral application include: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In some embodiments, the carrier is a solvent or dispersion medium containing, for example, water, ethanol, polyol (for example, glycerol, propylene glycol, and liquid polyethylene glycol, and the like), or suitable mixtures thereof. Fluidity is maintained, in some embodiments, for example, by the use of a coating such as lecithin, by the maintenance of the required particle size in the case of dispersion, and by the use of surfactants. Antibacterial and antifungal agents include, for example, parabens, chlorobutanol, phenol, ascorbic acid, and thimerosal. Isotonic agents, for example, sugars; polyalcohols such as mannitol or sorbitol; or sodium chloride, in some embodiments, are included in the composition. In some cases, also included is an agent which delays absorption, in some embodiments, for example, aluminum monostearate or gelatin prolongs absorption of injectable compositions.

In some embodiments, sterile injectable formulations are prepared by incorporating the active composition in the required amount in an appropriate solvent with one or a combination of the above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. In some embodiments, transmucosal administration is accomplished through the use of nasal sprays, inhalation devices (e.g., aspirators) or suppositories. For transdermal administration, the active compounds are formulated into ointments, salves, gels, creams or patches.

In some embodiments, the pharmaceutical formulations are prepared with carriers that protect against rapid elimination from the body, such as a controlled release formulation or a time delay material such as glyceryl monostearate or glyceryl stearate. The formulations, in some embodiments, are also delivered using articles of manufacture such as implants and microencapsulated delivery systems to achieve local, regional or systemic delivery or controlled or sustained release.

There remains significant unmet need for patients with NSCLC with targetable oncogenic mutations relapsing after TKIs, so we see a potential benefit in novel therapies that can be easily combined with established CPIs. Combining CLN-619 with pembrolizumab engages multiple immune effector cells, including innate cells by CLN-619 and T cells by pembrolizumab. The safety profile of CLN-619 along with the biologic rationale for combination with CPI make this a potentially synergistic approach.

Initial clinical findings show that the combination of CLN-619, a novel antibody targeting MICA/B, with pembrolizumab may benefit patients whose cancer is not typically amenable to checkpoint inhibitor therapy. More specifically, the inventors observed objective responses in patients with ALK- and EGFR-mutated NSCLC who had relapsed after tyrosine kinase inhibitors (TKIs), patients who do not typically respond to checkpoint inhibitors.

Additionally, longer-term follow-up for patients treated with CLN-619 monotherapy shows favorable safety and durable clinical benefit with extended treatment, including objective responses and prolonged stable disease in multiple tumor types and in patients with disease progression after CPI therapy.

### EXAMPLES

The following examples are given for the purpose of illustrating various embodiments of the invention and are not meant to limit the present invention in any fashion. The present examples, along with the methods described herein are presently representative of certain embodiments, are exemplary, and are not intended as limitations on the scope of the invention. Changes therein and other uses which are encompassed within the spirit of the invention as defined by the scope of the claims will occur to those skilled in the art.

### Example 1. CLN-619 (Anti-MICA/B Antibody) Promotes Innate Immune Cell Mediated Anti-Tumor Activity

CLN-619 is a humanized IgG1 monoclonal antibody that targets MICA and MICB (MICA/B) and is currently in phase 1 clinical development in cancer patients (NCT05117476; see Examples below). The present example shows that this antibody promotes innate immune cell anti-tumor activity.

MICA/B serves as activating signals on target cells for recognition by the NKG2D receptor, which is expressed on NK cells and a subset of T cell populations. MICA/B expression is induced in response to stressed conditions, thereby enabling NKG2D-mediated elimination of target cells. On NK cells, NKG2D is one of many receptors in the complex network of activating and inhibitory receptors, whereby NKG2D pathway activation results in cytokine production, enhancement of ADCC and target cell death. On CD8 T cells, the NKG2D axis plays a costimulatory role in lowering the threshold for lysis upon TCR engagement and may also drive direct CD8-mediated killing following prior TCR activation. MICA/B is expressed broadly on a range of solid and hematological malignancies. However, tumor cells evade NKG2D-mediated elimination by shedding MICA/B ligand from the cell surface via proteases present in the tumor microenvironment (TME). CLN-619 functions by binding to MICA/B and preventing shedding, thereby increasing MICA/B cell surface expression to restore the NKG2D-MICA/B axis to promote tumor cell killing by NK cells and T cells. Additionally, CLN-619 can mediate ADCC and ADCP by NK cells and macrophages. In preclinical xenograft models, doses as low as 0.03 mg/kg inhibited tumor outgrowth.

The inventors investigated the effects of CLN-619 on a variety of primary immune cells including NK cells, T cells and macrophages. In the evaluation of NK cells, CLN-619 modulation of cytokine production and cytotoxicity was measured in the presence of MICA/B expressing target cells. In addition, the present Example demonstrates the contribution of Fc-mediated functions of CLN-619. In the context of T cells, the requirement of TCR co-stimulation or prior antigen exposure in the activation of the NKG2D pathway by CLN-619 was explored. In the context of macrophages, the inventors measured the ability of CLN-619 to mediate ADCP against MICA/B-expressing target cells. These data highlight the potential of CLN-619 to engage multiple immune cell types within the TME, which may lead to greater and broader efficacy compared to IO therapies targeting a single immune cell type.

### A. Methods

CLN-619 was characterized for binding epitope and affinity, effects on surface and soluble levels of MICA/B, and in vitro tumor cell killing. In mouse models, the mAb was tested for tumor growth inhibition. The contribution of the functional Fc-gamma 1 (Fcγ1) domain of CLN-619 activity was also evaluated.

### Generation of CLN-619

To generate a diverse panel of monoclonal antibodies against human MICA/B proteins, a repetitive immunizations multiple sites (RIMMS) immunization protocol was carried out in Swiss James Lambert (SJL) mice. The full-length extracellular (ECD) of the most common allelic variant (MICA*008), was used as the antigen [Klussmeier A, et al., Front Immunol. 2020; 11]. The lead candidate was humanized using in silico modeling.

### Octet Binding Assessment of CLN-619 to MICA and MICB

To determine monovalent affinity, CLN-619 antibody (10 μg/ml) was captured on Dip and Reada Anti-Human IgG Fc Capture Biosensors. Sensors were then incubated with purified MICA*001, MICA*002, MICA*004, MICA*008, or MICB*004 ECD proteins. A fitting algorithm (ForteBio analysis suite 8.0), which assumed 1:1 binding, was used to calculate ka, kd, and KD. The BLOSUM62 algorithm was used to determine the similarity of the MICA/B proteins.

### ELISA Assessment of CLN-619 Binding to MICA/B Allelic Variants

96-well plates were coated with monomeric his-tagged MICA/B as the capture antigen, followed by the addition of a serial dilution of CLN-619. Unbound materials were washed away, and horseradish peroxidase (HRP) conjugated mouse anti-human IgG Fc was added to the wells for detection.

### Luminex Assessment of CLN-619 Binding to MICA

Luminex (LifeCodes LSA MIC Luminex Kit) was used to assess binding of CLN-619 (10 μg/mL) and 6D4, a positive control antibody, to 28 different allelic variants of MICA [Ghadially H, et al. Br J Cancer. 2017; 116:1208-17]. A goat anti-mouse secondary antibody was used to detect 6D4. Background MFI was subtracted from the raw MFI and duplicate samples were averaged.

### X-Ray Crystallography of MICA-CLN-619

X-ray diffraction data was collected from crystals of Fab CLN-619: MICA at the SWISS LIGHT SOURCE (SLS, Villigen, Switzerland) using optimized cryogenic conditions. The structure of the complex of human Fab antibody fragment CLN-619 with MICA alpha 3 domain (Fab CLN-619: MIC-A) was at 2.12 Å resolution.

### Flow Cytometry Assessment of Surface MICA/B Levels Following CLN-619 Treatment

HCC1534, PLC/PRF/5 and HCT-116 cells were incubated with serially diluted CLN-619, CLN-619 DANA or hlgG1 control antibody for 24 hrs. After washing, cells were stained with live/dead viability dye. Cell surface MICA/B was detected with 6D4 antibody, an anti-human MICA/B-PE antibody that is non-competitive with CLN-619. A mouse IgG2a-PE isotype antibody was used as a control. Quantibrite beads were used to quantify surface MICA/B levels. Dose response curves were fit using a 4-parameter logistic regression model.

### Assessment of Soluble MICA/B Levels Following CLN-619 Treatment

HCC1534, PLC/PRF/5 and HCT-116 were plated and treated with serially diluted CLN-619 or hIgG1 in duplicate for 24 hours. Supernatant was collected for analysis of sMICA levels in an enzyme linked immunosorbent assay (ELISA). Antibodies used in the ELISA were non-competitive with CLN-619. ELISA plates were coated with BAMO1 capture antibody. A standard curve of recombinant human MICA*001 ECD and supernatant samples were added to the plates. Biotinylated detection antibody 10E9.H6 was added to the plates followed by the addition of Streptavidin-Horse Radish Peroxidase (HRP). EC50 values were calculated using the four-parameter nonlinear fit model in GraphPad Prism.

### Primary NK Cell Killing Assay

The ability of CLN-619 to mediate NK cell mediated tumor cell killing was assessed in a co-culture assay of purified human primary NK cells and MICA-expressing HCC1534 tumor cells in a xCELLigence system. NK cells were isolated through negative selection from frozen PBMC stocks from healthy human donors and checked for purity. HCC1534 target cells were plated on an E-96 Xcelligence glass plate with serially titrated antibody. After HCC1534 cells adhered, NK cells were added at an effector to target cell ratio (E:T) of 40:1, and HCC1534 cell death was monitored over 72 hrs. EC50 values were calculated using the four parameter nonlinear fit model in GraphPad Prism.

### ADCC Reporter Assay

ADCC activity of CLN-619 was measured using a reporter assay (Promega). Jurkat cells expressing a high (V) or low affinity (F) FcγRIIIa were engineered to express a nuclear factor of activated T-cells (NFAT)-luciferase reporter construct. HCT-116 or HCC1534 cells were treated with a serial dilution of CLN-619 or IgG1 control antibody. Engineered Jurkat cells were added in an E:T ratio of 10:1 for 6 hours. EC50 values were calculated using the four-parameter nonlinear fit model in GraphPad Prism.

### Primary Cell ADCP Assay

CD14+ monocytes were collected from two healthy PBMC donors by magnetic positive selection using the RoboSep (protocol 17858) or manual isolation (StemCell). Macrophages were differentiated for 6 days then harvested and seeded with M-CSF supplemented media. HCC1534 or HCT-116 eFluor670 labeled target cells and macrophage effector cells were plated at an E:T ratio of 0.5:1 and incubated for 2 hours with serially diluted CLN-619 or hIgG1 or cetuximab control antibodies at the highest dose. After incubation, macrophages were successively incubated with viability dye, blocking buffer (Biolegend), and BV510 labeled anti-CD64 antibody. ADCP was determined via flow cytometry by gating target cell fluorescence (eF670+) within CD64+ cells.

### Flow Cytometry Assessment of MICA Binding to NKG2D on NK Cells.

NK cells were purified from PBMCs of a healthy donor by negative selection. Serially diluted CLN-619, CLN-619 DANA or hIgG1 antibody were preincubated with his-tagged MICA ECD at a ratio of 10:1. Antibody-MICA complexes were then added to the NK cells in the presence or absence of anti-NKG2D antibody. MICA bound to the NK cells was detected by anti-His antibody.

### Biacore Assessment of CLN-619 to Fc Gamma Receptors

To evaluate CLN-619 binding to Fc gamma receptors (FcγRs), recombinant His6-tagged FcγRs were immobilized onto an anti-His6 antibody coated CM5 chip. CLN-619 WT or CLN-619 DANA were flowed over the biosensor. Ka, kd and KD were calculated at steady state for all FcR's except for CD64, which was evaluated at 1:1 affinity.

### In Vivo Efficacy Studies

BALB/c SCID mice were inoculated with 10×106 HCC1534 cells. Mice were treated with hIgG1 (10 milligram per kilogram (mg/kg)), CLN-619 (or 0.03, 0.3, 3.0, 10 mg/kg), or CLN-619 DANA (0.03, 0.3, 3.0 mg/kg). Dosing began on the same day as the tumor cell implantation and continued two times a week for a total of 8 doses. BALB/c mice were inoculated subcutaneously with 5×106 PLC/PRF/5 cells. Mice were dosed IP with either vehicle (PBS) or CLN-619 (0.3 mg/kg or 3 mg/kg) three times a week for a total of 16 doses.

For both studies, tumor size and body weight were measured two times a week for the duration of the study. The HCC1534 and the PLCR/PRF/5 studies ended on Days 35 and 39, respectively. Statistical analysis was performed using two-way ANOVA with multiple comparisons (GraphPad).

HCT-116 MICA/B-expressing human colorectal cancer cells tagged with luciferase were inoculated i.p. Treatment began on Day 4 with hIgG1 or CLN-619 dosed at 10 mg/kg administered two times a week for five and six weeks by IP injection, respectively. Bioluminescent imaging was performed twice weekly to monitor progression of disseminated disease. Survival analysis was performed based on Day 40, when the final mouse reached a peri-moribund state. Statistical analysis was performed using a Log-rank test.

### Pharmacokinetics

BALB/c SCID mice were dosed i.p. with 1, 3 or 10 mg/kg of CLN-619, and blood samples were collected during the study at the following timepoints: 1, 3, 6, 24, 48, 72, 96 hr and at days 11, 14, 21, 28, by mandibular bleeds. A total of 3 or 4 mice were assessed at each time point in each dose group. Serum concentrations were measured via MICA/B sandwich ELISA.

### B. Results

CLN-619 bound with high affinity to the alpha 3 domain of MICA/B without encumbering the interaction with NKG2D on NK cells. CLN-619 increased the level of cell surface expression of MICA/B and concomitantly decreased the levels of soluble MICA/B in vitro. Treatment of cancer cell lines with CLN-619 induced antibody-dependent cellular cytotoxicity (ADCC) and antibody-dependent cellular phagocytosis (ADCP) with EC50 values dependent on MICA/B expression levels. CLN-619 resulted in potent inhibition of tumor growth in liver and lung mouse xenograft models and increased survival of mice in a disseminated colorectal cancer model.

### CLN-619 Binds to the Alpha 3 Domain of MICA/B

CLN-619 was derived from a parental murine antibody that was selected for high affinity binding to the alpha 3 domain of MICA and the ability to augment cell surface expression of MICA/B. The variable domains of the parental antibody were humanized and introduced into a human IgG1 backbone to generate CLN-619.

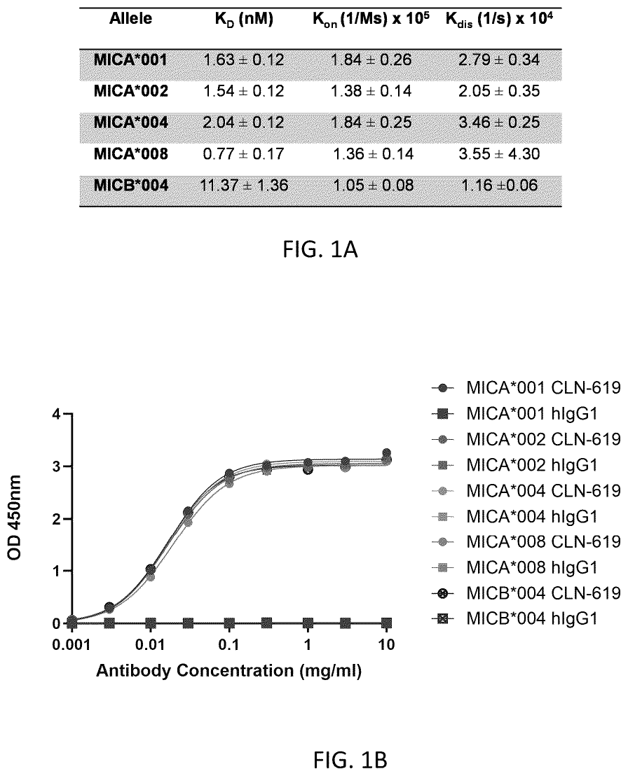

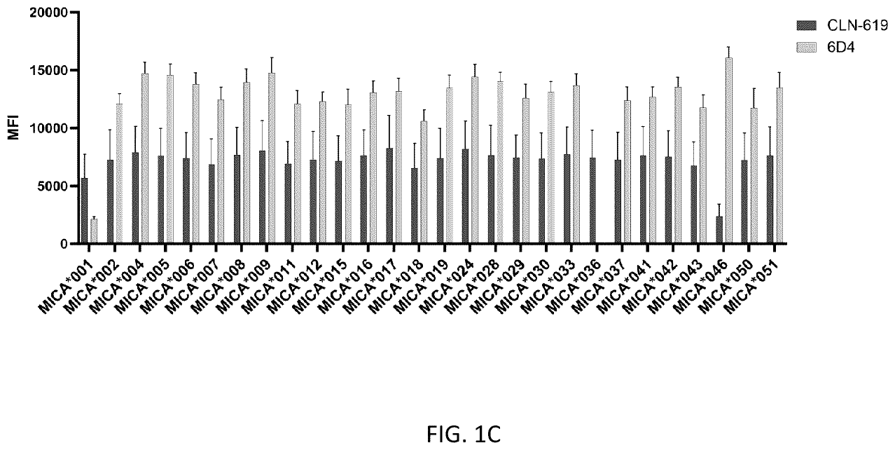

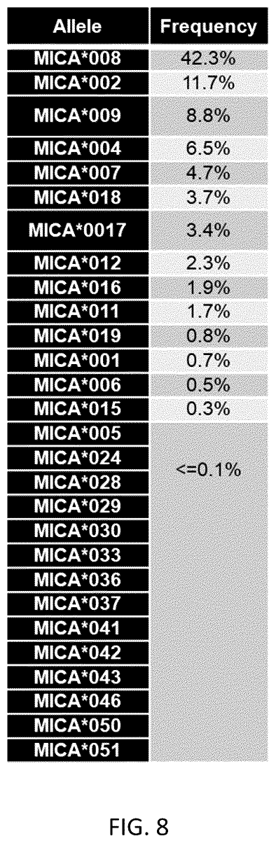

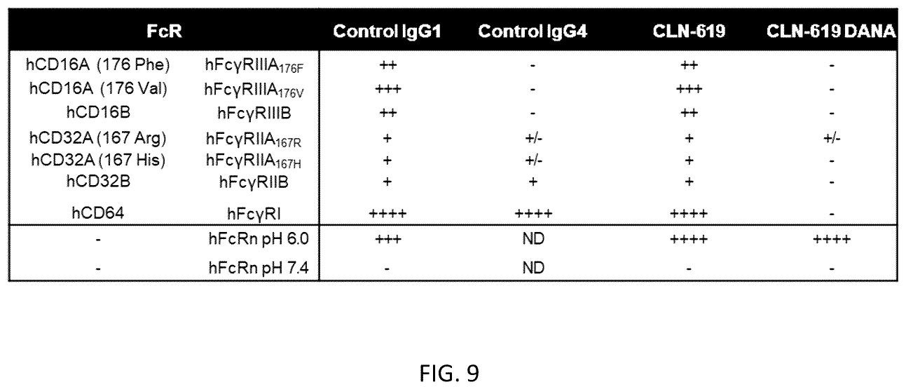

The MICA/B genes are highly polymorphic with nearly 150 alleles of MICA and 50 alleles of MICB present in the human population. The canonical MICA *001 and MICB *004 alleles are 83% identical in overall protein sequence and 91% identical in the alpha 3 domain. Given the highly polymorphic nature of the proteins, it was critical to confirm broad reactivity of CLN-619. The monovalent affinity of CLN-619 for the extracellular domain (ECD) of several common human MICA alleles (MICA *001, *002, *004, and *008), and the MICB allele *004 was measured by Octet. KD values for CLN-619 binding to recombinant MICA allelic variants ranged from 0.77 to 2.04 nM, i.e., all within three-fold (FIG. 1A). The KD value of CLN-619 binding to the MICB allele *004 was 11.37 nM, i.e., approximately 5- to 15-fold lower than for the MICA alleles. ELISA experiments independently confirmed binding of CLN-619 to the representative allelic variants and demonstrated similar binding affinities across the MICA and MICB alleles (FIG. 1B). The broad reactivity of CLN-619 was further confirmed in a Luminex-based assay assessing 28 recombinant human MICA ECD proteins representing the most prevalent alleles (FIG. 1C). CLN-619 showed a similar affinity for all alleles, except for MICA allele *046, a rare allele present in less than 0.01% of the population, where reduced binding relative to the other alleles was observed (FIG. 8). Overall, CLN-619 exhibited broad reactivity across all MICA alleles tested, which represent 89% of the population.

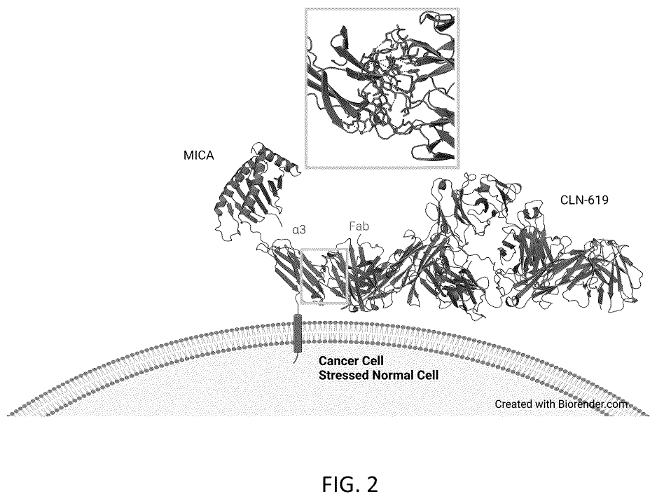

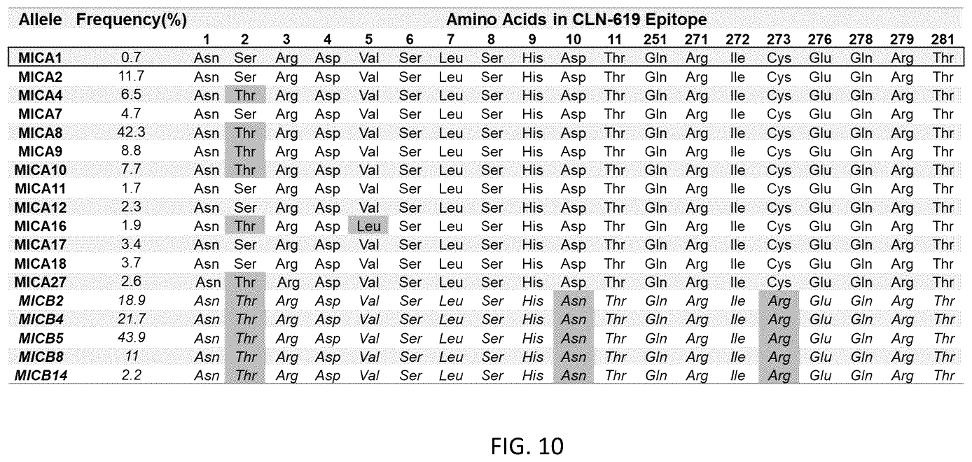

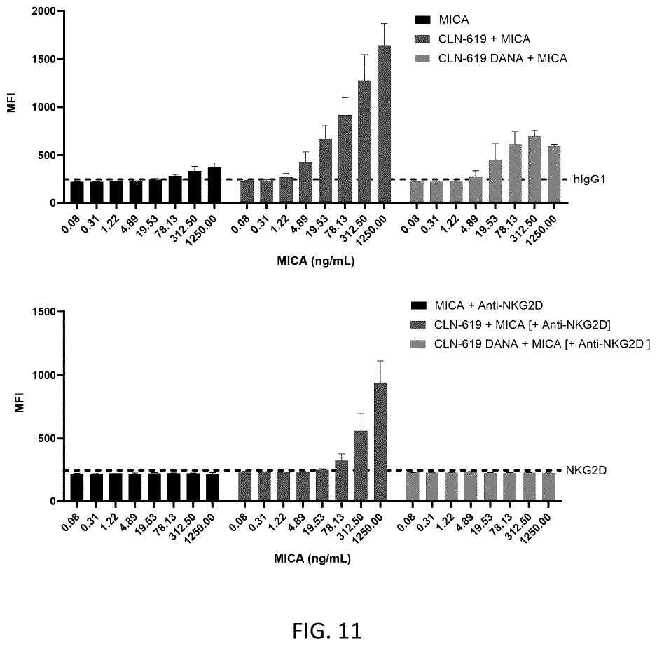

An x-ray crystallographic structural analysis was carried out to elucidate the interaction between CLN-619 and the alpha 3 domain of MICA at an atomic resolution. A co-crystal structure of the CLN-619 Fab fragment and the MICA alpha 3 domain of MICA allele *001 was obtained at a resolution of 2.12 angstroms. CLN-619 recognized a discontinuous epitope on the alpha 3 domain, defined as Thr204 to Ser297, interacting with a total of 19 amino acid residues that cover approximately 20% of the alpha 3 domain Notably, several of the known cleavage sites within the alpha 3 domain of MICA/B overlap with the epitope recognized by CLN-619. The CLN-619 epitope is highly conserved across the most common MICA allelic variants, with amino acid changes observed in only 4 of the 19 interacting amino acid residues, with most of these changes being relatively conservative (FIG. 10). Importantly, CLN-619 bound the alpha 3 domain of MICA in a region and by an angle that should not sterically interfere with binding of the alpha 1 and 2 domains to the NKG2D dimer (FIG. 2). Supporting the crystallographic analysis, CLN-619 did not hinder the NKG2D-MICA interaction in a flow-based assay (FIG. 11). Interestingly, when MICA was complexed with CLN-619, increased NK cell binding was observed compared to MICA alone, likely reflecting simultaneous engagement of CLN-619 with both NKG2D and CD16A on NK cells. When an anti-NKG2D antibody was included in the experimental conditions, MICA binding to primary NK cells was significantly reduced.

### CLN-619 Augments Cell Surface Expression of MICA/B on Tumor Cells

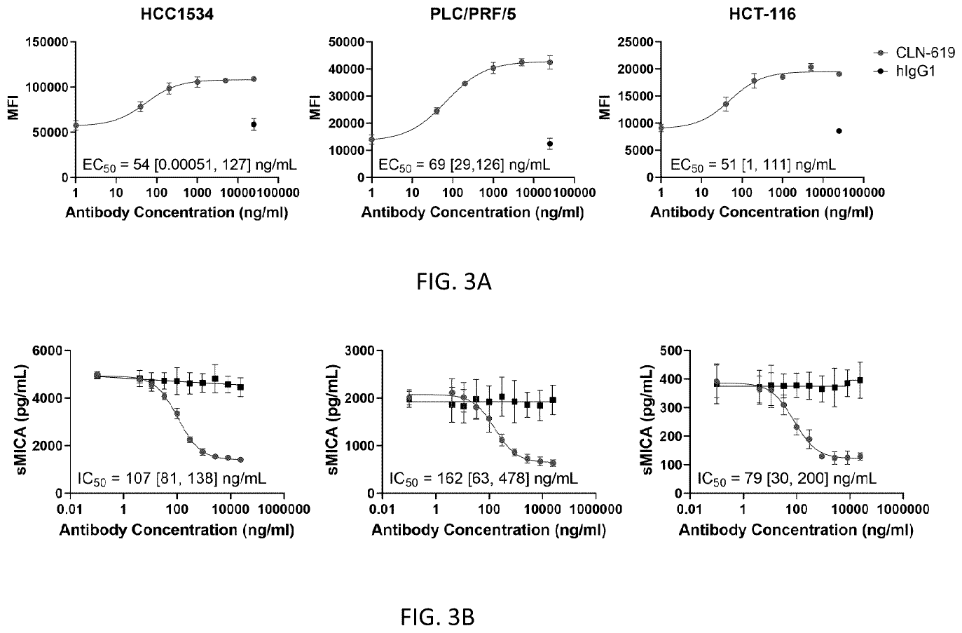

Based on the structural data, the inventors hypothesized that CLN-619 binding to MICA/B in the alpha 3 domain may prevent access of proteases to MICA/B ligands thereby resulting in reduced shedding and increased cell surface expression. We assessed the ability of CLN-619 to modulate the levels of cell surface and soluble MICA/B in cell-based assays using human cancer cell lines HCC1534 (lung), PLC/PRF/5 (liver), and HCT-116 (colorectal). These cancer cell lines express approximately 58,000, 17,000 and 10,000 copies of MICA/B on their cell surface, respectively, as measured by flow cytometry. Following treatment with CLN-619, cell surface levels of MICA/B were quantified by flow cytometry and sMICA/B levels present in culture media were measured by ELISA. CLN-619 but not control hlgG1 augmented the surface expression of MICA/B (FIG. 3A) and concomitantly decreased the levels of sMICA in a dose-dependent fashion (FIG. 3B). EC50 values for increased MICA/B surface expression were 54, 69 and 51 ng/mL CLN-619 for HCC1534, PLC/PRF/5 and HCT-116 cell lines, respectively. IC50 values for inhibition of sMICA/B release into the cell culture medium were 107, 162 and 79 ng/mL CLN-619, respectively, for the three cell lines. Notably, the similar EC50 values for enhancement of MICA/B on the cell surface by CLN-619 and IC50 values for reduction of sMICA/B in the supernatants support a causal relationship.

### CLN-619 Mediates NK Cell-Mediated Cytokine Production and Target Cell Lysis

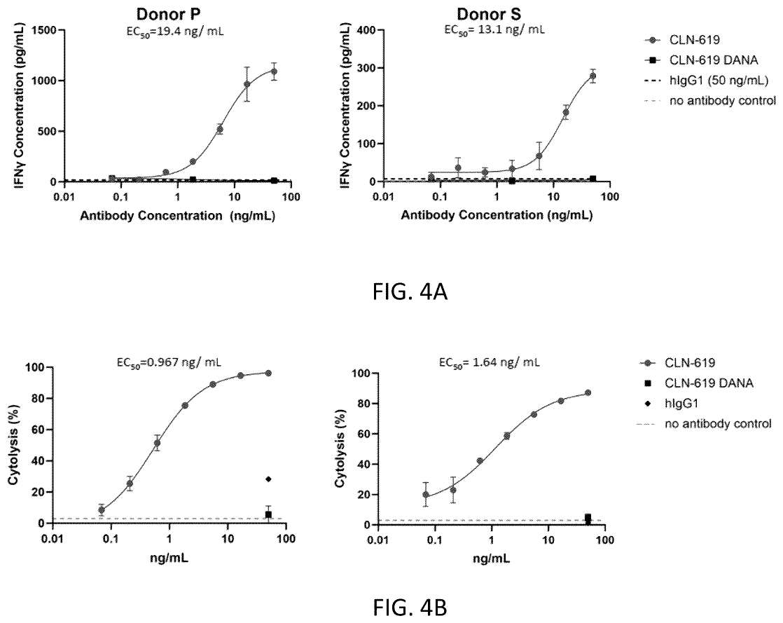

The ability of CLN-619 to stabilize the MICA/B ligands on the tumor cell surface suggested that CLN-619 may be capable of augmenting NK cell activation due to increased levels of ligand engaging the activating NKG2D receptor. The effect of CLN-619 on NK cell-mediated cytokine release and target cell lysis was evaluated in a co-culture assay using primary NK cells derived from two healthy PBMC donors in the presence of HCC1534 target cells. In a co-culture system comprising primary NK cells and MICA/B expressing tumor cells, CLN-619 induced a dose-dependent release of IFNγ and potent target cell lysis, with EC50 values of 13-19 ng/ml (FIG. 4A), and EC50 values of 1-2 ng/mL for target cell lysis (FIG. 4B). Activity was dependent upon an intact Fc-domain.

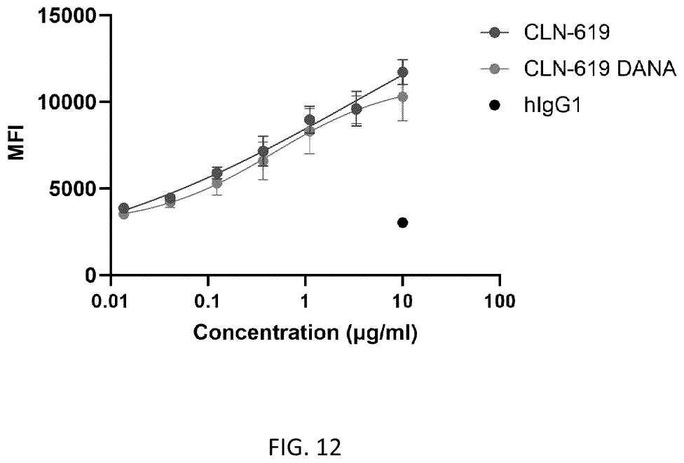

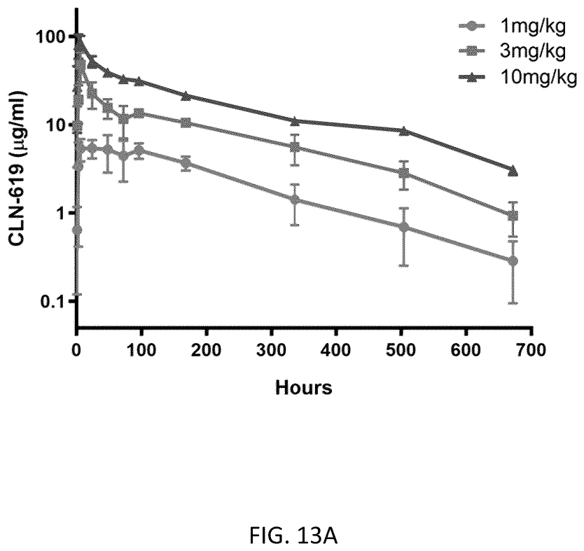

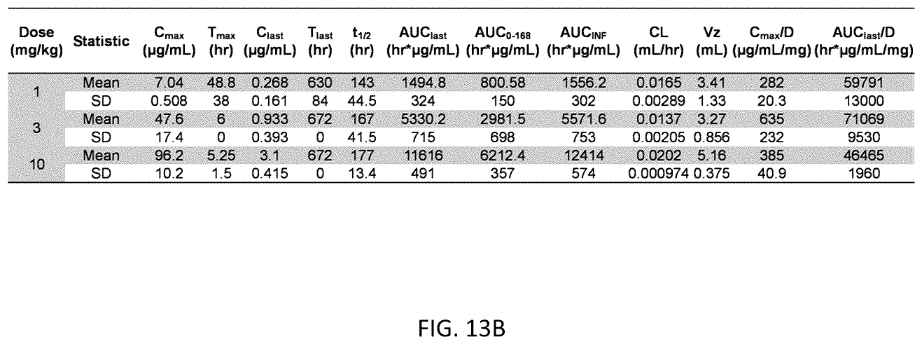

An Fc-silenced version of CLN-619, referred to as CLN-619 DANA, was generated to evaluate the contribution of FcγRyengagement to CLN-619-mediated NK cell activation and tumor cell killing. CLN-619 DANA contains two mutations in the Fc domain, D265A and N297A, previously shown to abolish FcγR binding of IgG1 antibodies. While the DANA mutation resulted in elimination of FcγR binding by CLN-619, the ability to bind to MICA and to modulate cell surface levels was retained (FIG. 9, FIG. 12). When evaluated in the primary NK cell co-culture assay, CLN-619 DANA was not capable of eliciting significant IFN release or target cell lysis (FIG. 4A, B). These results demonstrate the requirement of an intact Fcγ1 domain for CLN-619-mediated immune activation and target cell lysis by NK cells.

### CLN-619 Induces ADCC and ADCP Against MICA/B-Expressing Tumor Cells

Given that CLN-619 is a human IgG1 antibody with a functional Fc domain, we evaluated whether CLN-619 was capable of mediating various Fc-mediated functional activities, such as ADCC, ADCP and CDC. Functional engagement of FcγRIIIa/CD16A by CLN-619 was investigated in a cell-based reporter assay designed as a surrogate to evaluate ADCC activity. Jurkat cell lines with luciferase gene expression driven under control of the NFAT transcription factor in response to human FcγRIIIa/CD16A activation were used as reporter effector cells. The Jurkat cell lines, one expressing the higher affinity V158 variant of CD16A and the other expressing the lower affinity F158 variant, were co-cultured with MICA/B expressing target cells in the presence of CLN-619. The strongest CD16A signaling was observed upon co-culture with HCC1534, the tumor cell line expressing high levels of MICA/B (FIG. 5). The EC50 values ranged from 8-81 ng/ml with CLN-619 treatment, with the signal being consistently lower for the F variant compared to the V variant of CD16A. Consistent with its functionally deficient Fc domain, CLN-619 DANA was inactive in the Jurkat cells assays. Overall, EC50 values for CD16A activation by CLN-619 in the reporter cell lines correlated with expression levels of MICA/B on target cells, suggesting that CLN-619 can mediate ADCC in a target-dependent manner.

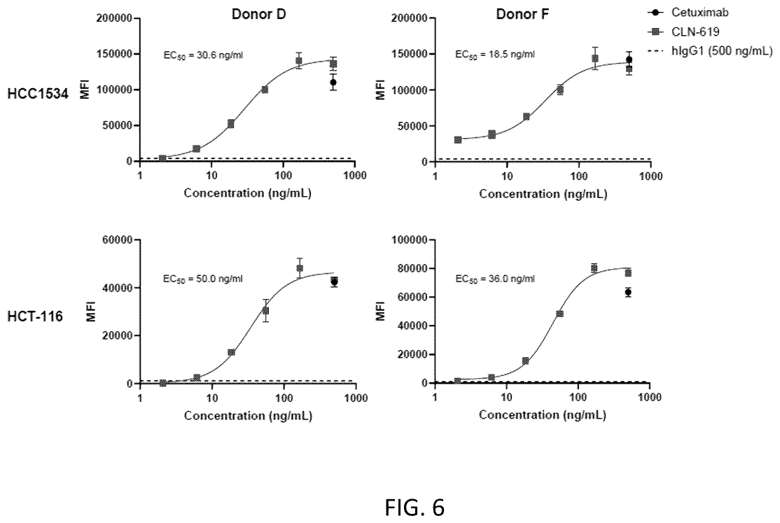

Given the ability of CLN-619 to also bind FcγRIIA/CD32A, the FcγR receptor primarily responsible for ADCP, CLN-619 was also evaluated for its ability to elicit macrophage mediated phagocytosis [20]. Human peripheral macrophages that had been polarized to the M1 phenotype were co-cultured with fluorescently-labeled HCC1534 or HCT-116 tumor lines. Phagocytosis in response to CLN-619 was measured by flow cytometry. The anti-EGFR mAb cetuximab was included as a positive control, as both cell lines were confirmed to express EGFR. Treatment with CLN-619 resulted in dose-dependent phagocytosis of labeled tumor cells similar to cetuximab, while minimal phagocytosis was observed with an isotype control antibody (FIG. 6).

While CLN-619 was capable of mediating ADCP and ADCC, no complement-dependent cytotoxicity (CDC) activity was observed in response to CLN-619 treatment (data not shown). However, CLN-619 treatment resulted in dose-dependent phagocytosis of labeled MICA/B expressing tumor cells in a co-culture assay with primary macrophages.

### Anti-Tumor Activity of CLN-619

Given the potent anti-tumor cell killing activity of CLN-619 observed in vitro, the effect of CLN-619 on tumor growth in vivo was assessed with human tumor xenografts expressing MICA/B in immunodeficient BALB/c SCID mice. Exposure of CLN-619 following intraperitoneal (i.p.) administration in BALB/c SCID mice was dose-proportional, and the serum half-life was in line with that of a typical human IgG1 antibody dosed in mice.

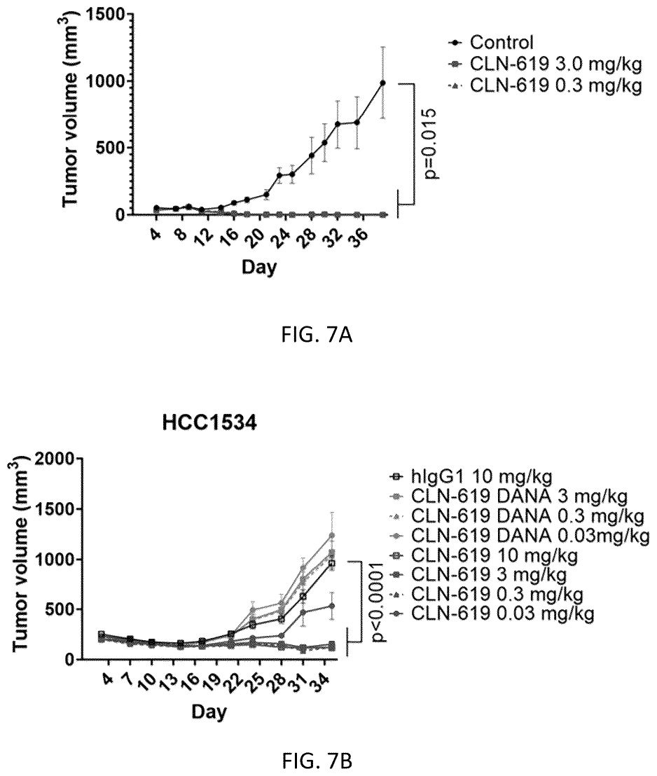

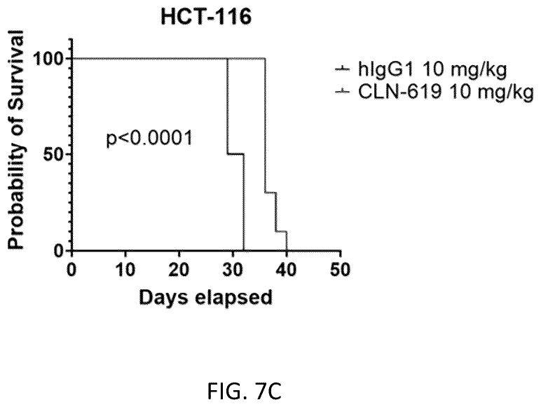

In the PLC/PRF/5 liver tumor xenograft model, i.p. administered CLN-619 at either 0.3 mg/kg or 3 mg/kg led to complete inhibition of tumor growth (p=0.015) compared to the control group (FIG. 7A). In the HCC1534 lung xenograft model, i.p. administered CLN-619 at dose levels ranging from 0.3 to 10 mg/kg demonstrated statistically significant (p<0.0001) dose-dependent anti-tumor activity compared to treatment with isotype control mAb, with tumor growth inhibition (TGIs) ranging from 45 to 87%. No anti-tumor activity was observed with CLN-619 DANA at any dose level (FIG. 7B). This data suggests that the intact Fcγ1 domain of CLN-619 is indispensable for anti-tumor activity in vivo, consistent with our in vitro observations in NK cell co-culture assays.

The activity of CLN-619 in a metastatic setting was explored in a mouse model designed to mimic late stage, disseminated disease. HCT-116 human colorectal cancer cells expressing luciferase were inoculated i.p. into mice allowing for tumor cell seeding to distal organs. Kaplan-Meier survival analysis demonstrated that CLN-619 treatment resulted in a statistically significant survival benefit as compared to hlgG1 (p<0.0001) (FIG. 7C).

### C. Conclusions

CLN-619 inhibits the shedding of MICA/B to effectively restore cytotoxic signaling pathways in immune cells. Potent anti-tumor activity of CLN-619 as a monotherapy was observed in several preclinical models. Activity of CLN-619 required a functional Fcγ1 domain, suggesting the requirement of simultaneous engagement of NKG2D and CD16A on immune cells for optimal cytotoxicity. The preclinical data reported in this Example support the assessment of CLN-619 in cancer patients.

The data shown herein revealed a multi-modal mechanism of action of CLN-619. Apart from inhibition of MICA/B shedding and increased expression of the NKG2D ligands on cancer cells, we observed ADCC and ADCP functionality of CLN-619 that was dependent on a wildtype hIgG1 backbone. A Fc-deficient version of CLN-619 did not show activity in the NK co-culture killing assay (FIG. 4B), despite retention of NKG2D binding (FIG. 11). CLN-619 DANA also lacked activity in-vivo. We hypothesize that dual stimulation of CD16A and NKG2D receptors on NK cells may be required for the activity of CLN-619. This concept of dual receptor engagement is supported by published studies showing that NKG2D engagement can synergistically enhance ADCC and augment CD16A and NKp46 activation of resting NK cells.

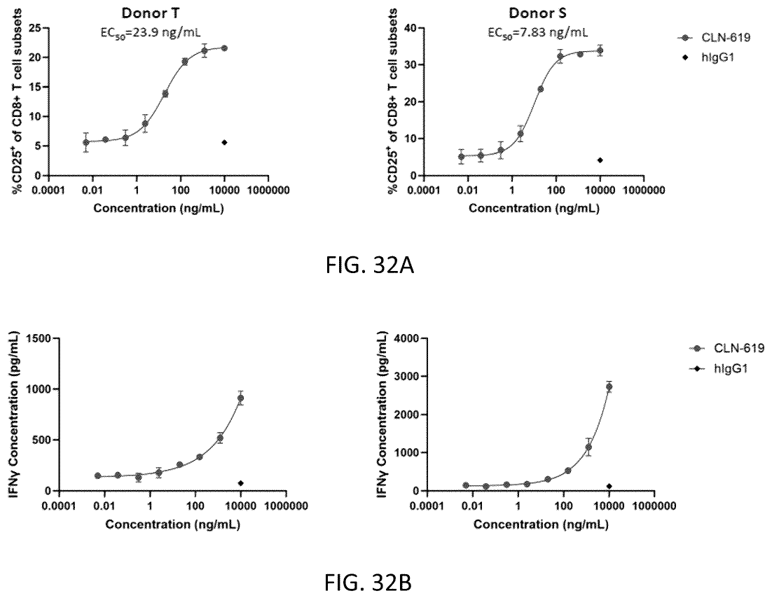

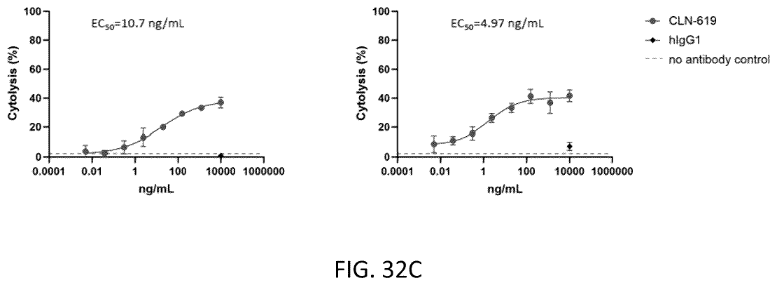

CLN-619 showed compelling single-agent activity at low dose levels in multiple tumor xenograft models using tumor cell lines representing indications where the NKG2D pathway has been shown to be clinically relevant, such as HCC and NSCLC. Human xenograft models were chosen because mice have no ortholog of MICA/B, while murine NKG2D on NK cells has been shown to recognize MICA/B on human tumor cells. The host mice used in our studies have competent NK cells and macrophages but are devoid of T cells. Therefore, the observed anti-tumor efficacy is most likely attributable to target cell lysis by NK cells and/or from phagocytosis by macrophages. A contribution to efficacy by NKG2D-expressing T cell subtypes, such as CD8+ T cells, NKT cells, and γδ T cells, would only manifest in fully immunocompetent model. Notably, the NKG2D axis has been shown to play a costimulatory role in lowering the threshold for lysis by CD8+ T cells and can even drive CD8+ T cell-mediated lysis of cancer cells following prior TCR activation in the absence of MHC expression. CLN-619 can likely engage a variety of cytotoxic immune effector cells in the TME to promote anti-tumor activity. In a co-culture system comprising an enriched population of CD8+ T cells and MICA/B expressing tumor cells, CLN-619 induced a dose-dependent release of IFNγ, upregulation of the CD25 marker of T cell activation, and target cell lysis. (FIGS. 32A-32C).

Therapies that potently engage NK cells may have advantages over therapies that exclusively engage T cells. NK cells do not require antigen priming for activation nor do they secrete high levels of cytokines that could trigger CRS, and they do not elicit graft-versus-host (GVH) reactions. However, NK cells may require more than one activating stimulus to fully unleash their killing potential and to overcome inhibition by the many negative regulatory receptors expressed on NK cells. Moreover, NK cells are heterogeneously distributed in the TME, and different indications may have variable numbers of infiltrated NK cells. In several indications, a dysfunctional subset of NK cells was found to be enriched in the TME and correlated with an unfavorable prognosis. A mechanism to improve NK cell performance may be the simultaneous engagement of synergistic NK cell-activating receptors, as described herein for CLN-619. Additionally, combination with therapies that enhance NK cell function, persistence and/or tumor infiltration could be beneficial and should be explored further.

Taken together, the data reported herein support the clinical investigation of CLN-619 for the treatment of a broad range of tumor types. A phase I clinical trial of CLN-619 in cancer patients as a monotherapy and in combination with pembrolizumab is described in further detail below (NCT05117476).

### Example 2. a Phase 1 Dose-Escalation Study to Investigate the Safety, Efficacy, Pharmacokinetics, and Pharmacodynamic Activity of CLN-619 (Anti-MICA/B Antibody) in Patients with Advanced Solid Tumors

Background: This example describes an ongoing a phase 1, multicenter, open-label, first-in-human dose-escalation study (NCT05117476) evaluating the use of CLN-619 as a monotherapy to treat patients with advanced solid tumors who progressed on available therapies, were intolerant to treatment, or refused noncurative standard treatment. CLN-619 is a humanized MICA/B-specific IgG1 monoclonal antibody that binds to and prevents proteolytic cleavage of NKG2D ligands MICA and MICB from tumor cells, thereby increasing tumor cell lysis by innate and adaptive immune cells. The amino acid sequence of CLN-619 is provided herein in Table 1. CLN-619 is expected to have broad anti-tumor activity.

Methods: This study was designed to characterize the safety, tolerability, dose-limiting toxicities, and preliminary antitumor activity of intravenously administered CLN-619 monotherapy in patients with advanced solid tumors. Patients aged 18 years or older having metastatic or locally advanced solid tumors that progressed after prior therapy and for whom no further standard treatments are available, an ECOG score of 0 or 1, and an estimated life expectancy of greater than or equal to 12 weeks were enrolled if they presented with measurable disease based on RECIST v1.1 and exhibited adequate liver and kidney function and hematologic parameters.

Patients who: (i) received investigational therapy within 25 days (or 5 half-lives) of the first scheduled dose of CLN-619; (ii) exhibited serious and/or uncontrolled medical disorder, including active autoimmune disease requiring immunosuppressive medication; (iii) received treatment for acute infection within 7 days of the first scheduled dose of CLN-619; (iv) had grade ≥3 immunological AE with prior checkpoint inhibitor therapy; or (v) active CNS metastases were excluded from the study.

CLN-619 alone (dose levels (DL) of 0.1 mg/kg, 0.3 mg/kg, 1 mg/kg, 3 mg/kg, 6 mg/kg, or 10 mg/kg) was administered intravenously over one hour every three weeks (Q3W).

Standard pre-medications (e.g., acetaminophen or ibuprofen) were given 30-60 minutes prior to each dose of CLN-619 and prior to each dose and per institutional practice thereafter.

Corticosteroid pre-medication for infusion related reaction (IRR) prophylaxis was required starting at the 3 mg/kg DL.

A parallel dose-escalation is exploring CLN-619 (DL 1.0 mg/kg and higher) in combination with pembrolizumab (as outlined in Example 2, below).

Response (RECIST 1.1) was assessed every 9 weeks. Patients are permitted to continue therapy until progressive disease, intolerable toxicity, or a maximum of 34 cycles. Patients with progressive disease may continue treatment if clinical benefit and acceptable tolerability is determined.

Study endpoints include: adverse events (CTCAE v5.0), Dose Limiting Toxicity (DLT), response (RECIST v1.1), and PK Parameters (e.g., Cₘₐₓ, AUC₀₋₅₀₄ₕ, t_{1/2}).

Data from the monotherapy dose escalation are described herein.

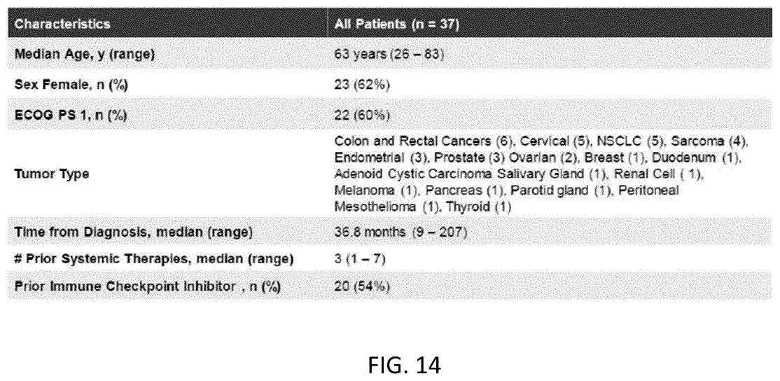

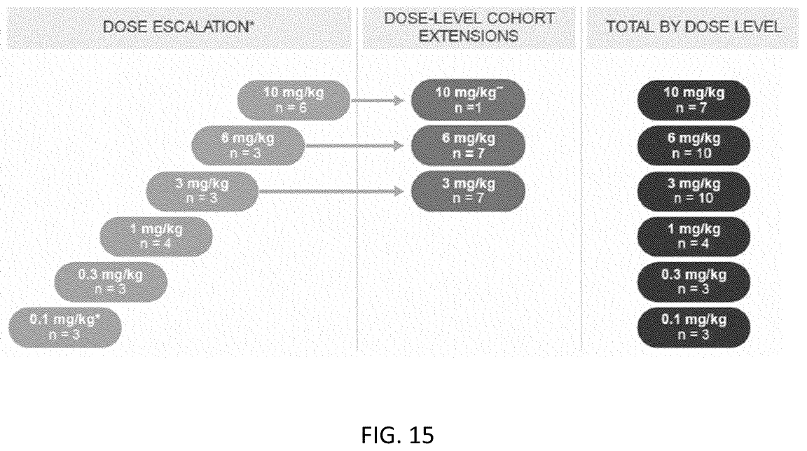

Results: Baseline patient characteristics are shown in FIG. 14 and the number of patients within each dose level is shown in FIG. 15. Briefly, 37 patients (median age, 63 y, range 26-83) were enrolled, 62% were female and 60% had ECOG PS 1. Tumor types included colon and rectal cancers (6), cervical (5), NSCLC (5), sarcoma (4), endometrial (3), prostate (3), ovarian (2), breast (1), duodenum (1), adenoid cystic carcinoma salivary gland (1), renal cell (1), melanoma (1), pancreas (1), parotid gland (1), peritoneal mesothelioma (1), and thyroid (1). All patients received prior systemic therapy (median 3, range 1-7); 20 patients had prior checkpoint inhibitor. The median number of CLN-619 cycles received was 2 (range, 0-13).

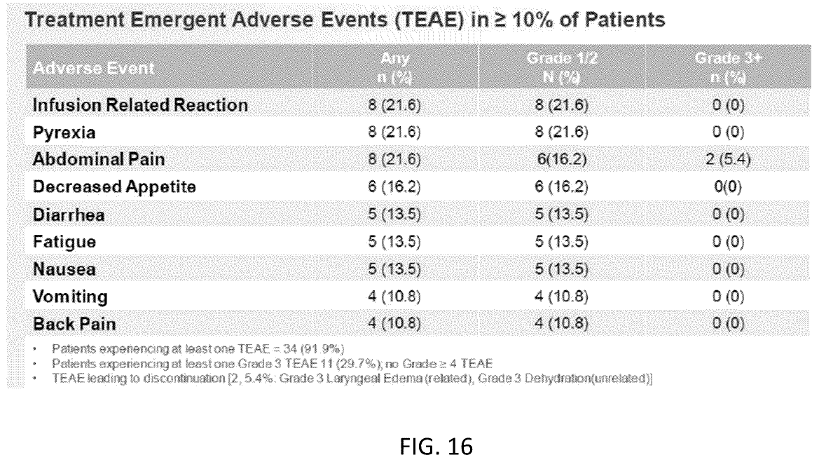

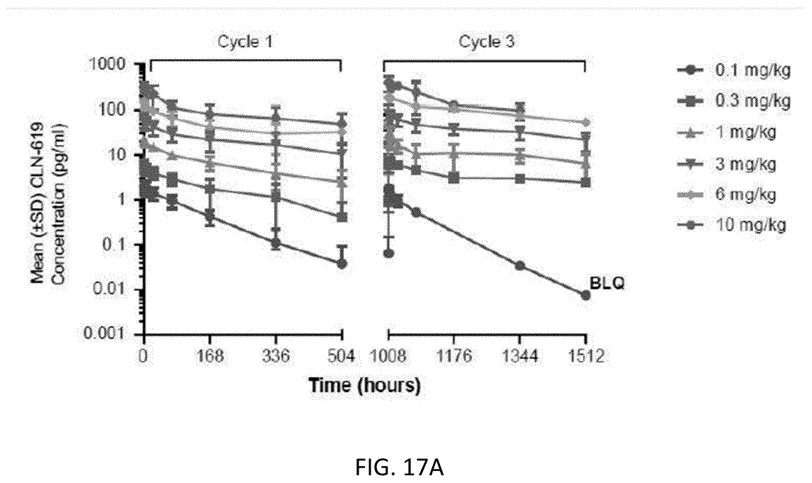

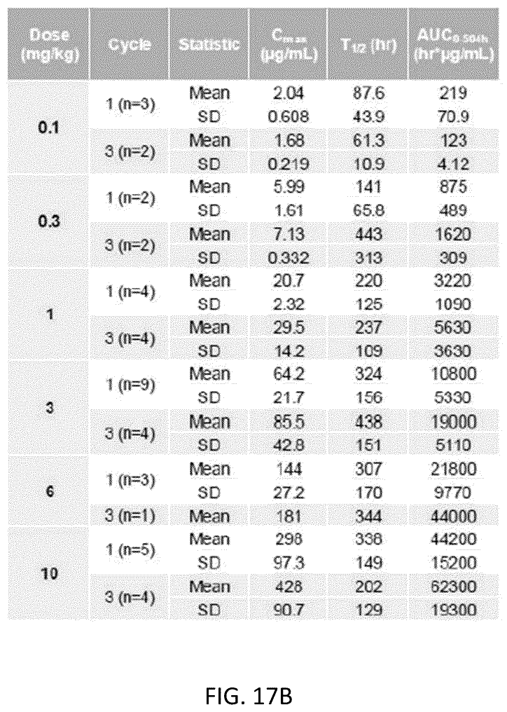

Treatment-emergent adverse events (TEAEs) were monitored and it was observed that CLN-619 was well-tolerated and most of the TEAEs were only at Grade ½. The TEAEs that were seen in ≥10% of patients (described in FIG. 16) ranged from abdominal pain, nausea, pyrexia, infusion related reaction (IRR), decreased appetite, fatigue, vomiting, and back pain. IRRs occurred at dose levels of 0.3 mg/kg and higher. All IRR occurred in Cycle 1 and resolved within 3 hours of onset. One case of Gr3 laryngeal edema occurred at the 10 mg/kg DL in the absence of required steroid premedication and led to dose discontinuation. No fatal TEAEs were reported, and no Adverse Event met the protocol-defined DLT criteria. These data showed the safety of CLN-619 in a clinical setting. As shown in FIGS. 17A and 17B, nearly dose-proportional increase in CLN-619 exposure, as measured by Cmax, was observed between the 0.1 mg/kg and 10 mg/kg dose levels. More than dose proportional increase in CLN-619 exposure as measured by AUC0-504h, was observed between 0.1 and 1 mg/kg and nearly dose-proportional increase in CLN-619 exposure was observed between 1 and 10 mg/kg dose levels.

CLN-619 half-life ranged from 61.3 to 443 hours for DL ranging from 0.1 mg/kg to 10 mg/kg.

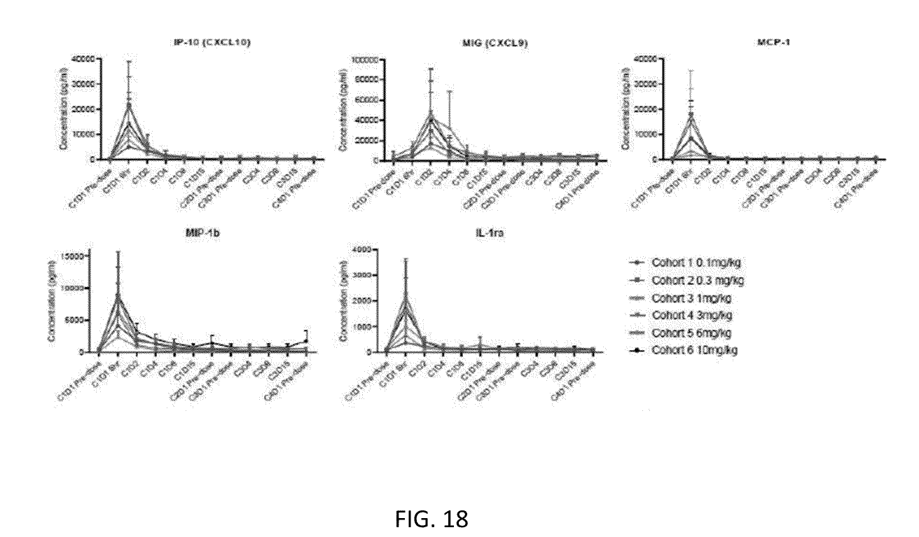

Cytokine expression in the longitudinal serum samples from patients treated with CLN-619 monotherapy was tested using Myriad RBM Inflammation and custom MAPs on the Luminex platform. The data are shown in FIG. 18, which depicts the average absolute cytokine levels (pg/ml) per dose level over time. Error bars are shown depicting SD. As shown in FIG. 18, transient increases in cytokine levels were observed at 2-6 hours after the first dose of CLN-619 and decreased to baseline by days 4-8. No dose-dependent increase in cytokine secretion was observed at the periphery.

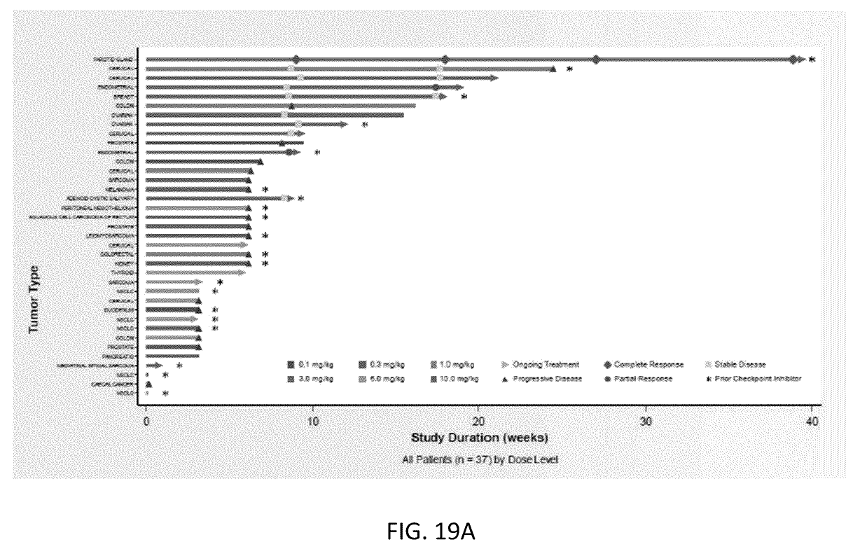

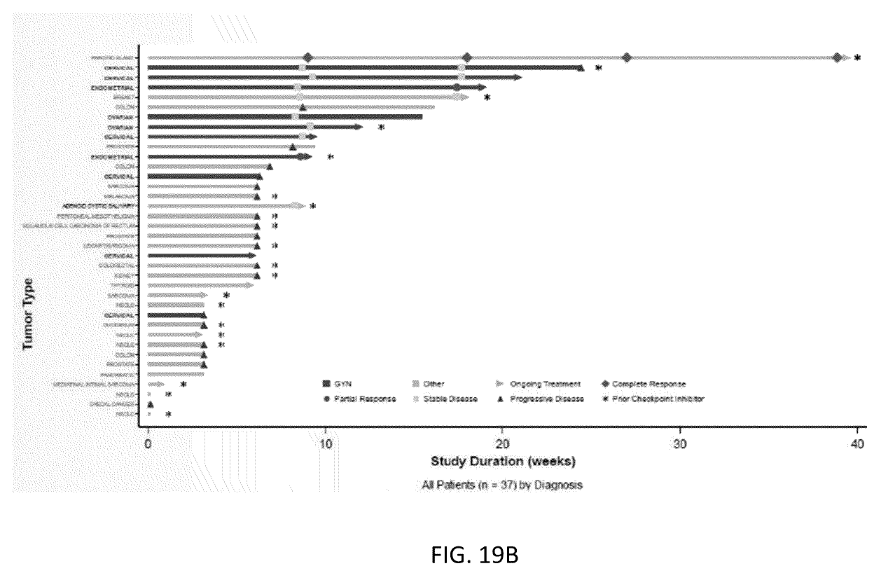

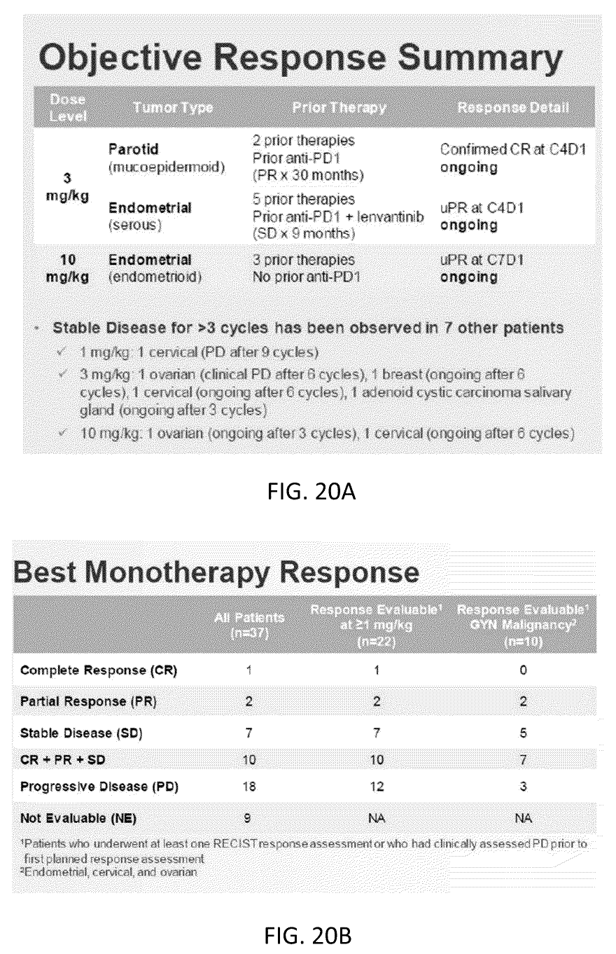

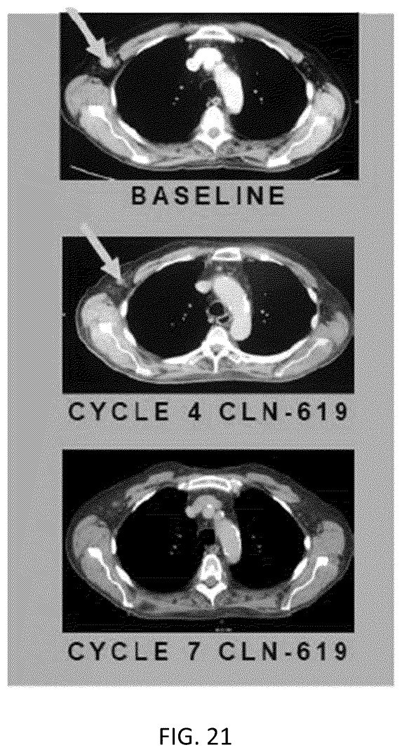

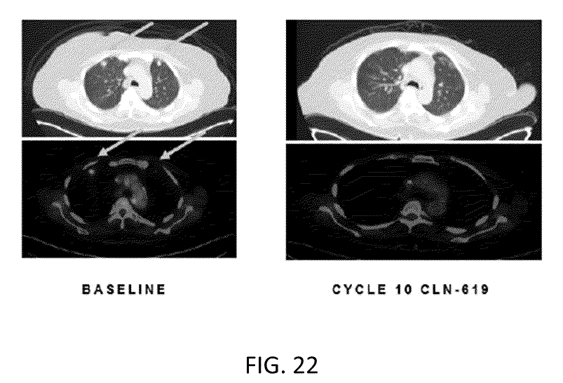

Time on treatment and clinical activity is illustrated in FIGS. 19A and 19B. Objective response summary and best monotherapy responses are outlined in FIGS. 20A and 20B, respectively. There was a confirmed complete response in a patient with recurrent salivary gland tumor who progressed following a complete response on a PD-1 blocking agent (FIG. 21). Data presented in FIG. 22 confirmed an objective response in a patient with endometrial cancer and a large tumor burden who progressed following platinum chemotherapy and anastrazole treatment. Stable disease for greater than 3 cycles has been observed in 7 other patients. For example, one patient with cervical cancer maintained SD for 9 cycles before progressing.

Conclusions: CLN-619 therapy was well tolerated at doses ranging from 0.1 to 10 mg/kg, and there were no dose-limiting toxicities observed at any of the doses administered, thereby demonstrating monotherapy clinical activity for CLN-619. Moreover, there was an objective response seen in a patient progressing after PD-1. No Grade ≥3 TRAE were observed in patients who received protocol-mandated pre-medications. Single agent activity, including objective responses, has been observed across multiple tumor types in both checkpoint-experienced and checkpoint-naïve patients. Notable single-agent clinical activity was observed in multiple gynecological malignancies demonstrating surprising efficacy in these subjects leading to expansion cohorts in endometrial and cervical cancers.

### Example 3. a Phase 1 Dose-Escalation Study to Investigate the Safety, Efficacy, Pharmacokinetics, and Pharmacodynamic Activity of CLN-619 (Anti MICA/MICB Antibody) Alone and in Combination with Pembrolizumab in Patients with Advanced Solid Tumors

The examples above demonstrate surprising and unexpected safety and efficacy of an anti-MICA/B antibody as a pan-cancer therapeutic agent as a monotherapy. The present example further shows the potential of this therapeutic agent in combination therapies. There are currently no approved products which target the NKG2D ligand/receptor pathway. CLN-619 targets this key pathway and represents a novel approach in 77mmune-oncology with the potential to benefit cancer patients in the future.

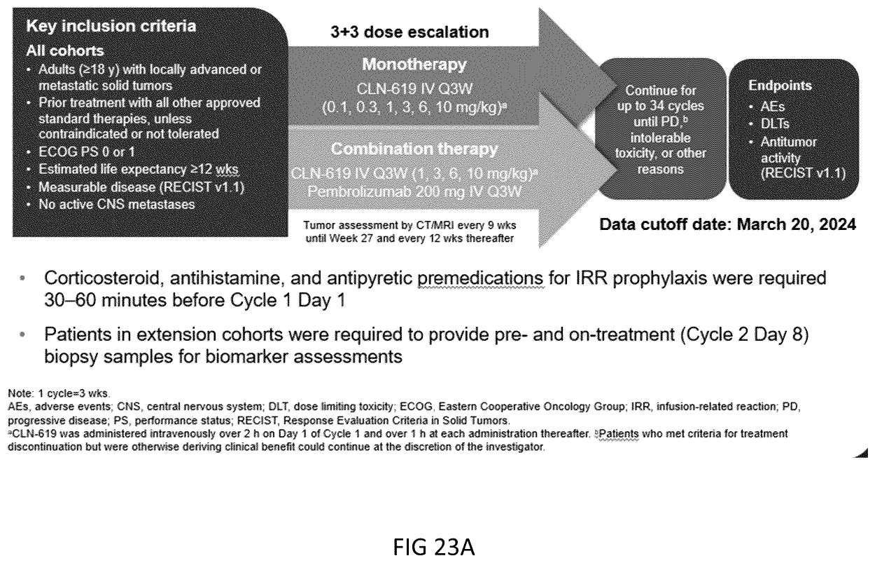

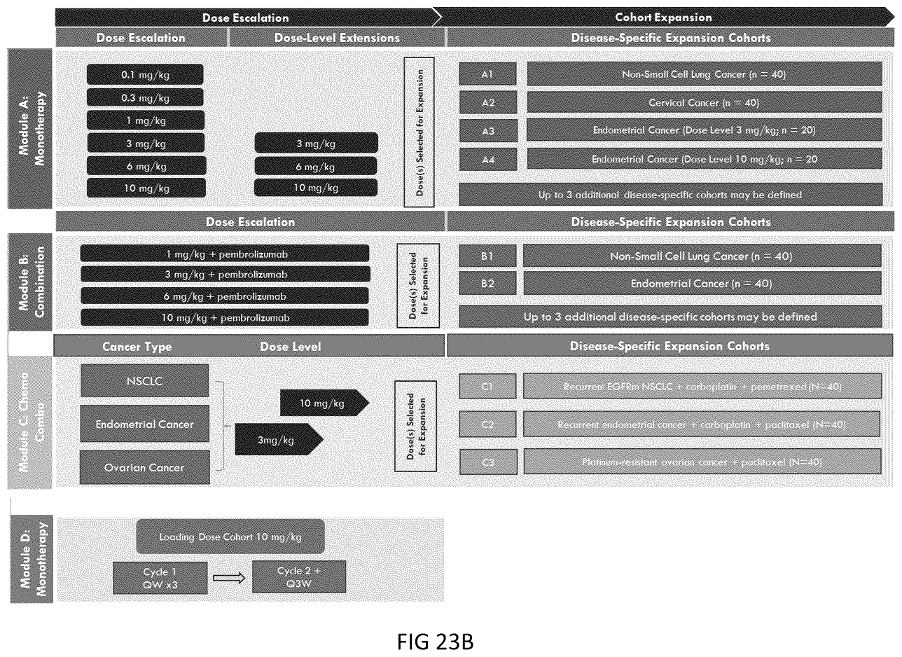

Study Description: Phase 1 open-label, first-in-human, multi-center, dose escalation and dose expansion study of CLN-619 administered alone (Module A and Module D) or in combination with pembrolizumab (Module B) or in combination with chemotherapy (Module C) in patients with select advanced solid tumors. See FIGS. 23A and 23B for overall study design.

CLN-619 will also be administered weekly during Cycle 1 to study a loading dose schedule.

Pembrolizumab is an anti-PD-1 antibody that will be administered to patients as a 30-minute IV infusion at 200 mg Q3W.

### Chemotherapies to be Administered Include:

Carboplatin AUC 5 and pemetrexed 500 mg/m2 Q3W IV,
Carboplatin AUC 5-6 Q3W and paclitaxel (175 mg/m2 Q3W) IV, and
Paclitaxel (60-80 mg/m2 Q1W) IV.

### Clinical Studies:

As of 22 Jun. 2023, CLN-619 is the only antibody targeting MICA/MICB that has been advanced into clinical trials. In the current study, a first-in-human investigation of CLN-619, multiple doses of CLN-619 (maximum dose of 10 mg/kg) have been administered every three weeks either alone or in combination with pembrolizumab to 61 patients. CLN-619 has been tolerated in both the monotherapy and combination therapy settings with no dose-limiting toxicities observed during the dose escalation and no Grade 3 treatment related Aes in patients who have received protocol-mandated pre-medications. At the time of analysis, cumulatively 235 doses of CLN-619 were administered, among those 52 doses in combination with pembrolizumab. Monotherapy expansion cohorts have been initiated in cervical and endometrial cancer and are currently ongoing.

Pembrolizumab. Pembrolizumab, marketed as Keytruda® in the United States, is a humanized IgG4 anti-PD-1 monoclonal antibody manufactured by Merck for the treatment of cancer. Pembrolizumab acts as an immunomodulator by blocking the interaction between the programmed cell death 1 (PD-1) receptor on activated T cells and its ligands, PD-L1 and PD-L2, which are expressed on tumor cells and immune cells.

Rationale for CLN-619 in Combination with Pembrolizumab

There is strong rationale for coordinated targeting of both innate and adaptive immunity with combined administration of CLN-619 and pembrolizumab. CLN-619 can enhance NKG2D mediated killing by both NK and selected T cell populations, and also potentiate antibody-dependent cellular cytotoxicity, an important cytolytic mechanism mediated by NK cells among other innate immune cell populations. In turn, pembrolizumab can enhance T cell mediated immunity by disrupting T cell exhaustion mediated by PD-1 and its ligands, PD-L1 and PD-L2. More recently, evidence suggests that the PD-1/PD-L1 axis may also play a role in the regulation of NK cell function (Dunai and Murphy 2018). Thus, by virtue of their respective modes of action, CLN-619 and pembrolizumab could both potentiate aspects of innate as well as adaptive immunity.

In the clinical setting, data suggests the potential for synergy between CLN-619 and checkpoint inhibitors (CPI). Among melanoma patients treated with CPIs, for example, patients with detectable levels of sMICA/sMICB have reduced overall survival (OS) compared to sMICA/sMICB-negative patients (Maccalli 2017). If MICA/MICB levels are stabilized on the tumor surface by CLN-619 treatment, CPI therapy may thus be more efficacious. It has additionally been hypothesized that persistent NKG2D signaling can lead to a PD-L1-rich tumor microenvironment, suggesting that NKG2D-modulating therapeutics would benefit from combination with PD-1 pathway inhibitors (Sheppard 2018).

### Chemotherapies

There are several types of chemotherapy, differing in their mechanism of action. The types of chemotherapies that will be administered during this study are briefly described below.

Platinum Agents (Carboplatin). Platinum compounds have two main mechanisms of action for their cytotoxic effects. They are alkylating agents that bind and cross-link DNA strands, thereby inhibiting DNA synthesis and function. If the DNA is damaged enough, the cell will undergo apoptosis. Platinum chemotherapeutics can also induce a type of cell death that is immunogenic and independent of DNA-binding effects. They interact with many cellular proteins, thereby modulating several signal transduction pathways.

Taxanes (Paclitaxel). Taxanes exert their anticancer effect by binding to the microtubule spindle machinery and prevent their depolymerization. This results in the blockage of metaphase-anaphase transitions, and ultimately, the inhibition of mitosis and induction of apoptosis. Unlike other microtubule disrupting drugs (e.g., the vinca alkaloids), paclitaxel specifically stabilizes microtubules by binding to the polymeric tubulin, thereby preventing tubulin disassembly. The broad-spectrum activity of paclitaxel was predicted by this mechanism of action (i.e., control of cell proliferation and DNA repair), which targets the very basic elements of the cancer phenotype.

Pemetrexed. Pemetrexed is a novel multitargeted antifolate that inhibits enzymes involved in folate metabolism and purine and pyrimidine synthesis. These precursors prevent DNA and RNA formation which are necessary for both normal and cancer cell growth and survival.

Rationale for CLN-619 in Combination with Chemotherapy

There is strong rationale for combining CLN-619 with standard of care chemotherapeutic agents. Cytotoxic chemotherapies such as platinums, taxanes and folate antimetabolities have been demonstrated to induce cellular stress resulting in the upregulation of NKG2D ligands including MICA/B. Additionally, these agents have been shown to positively impact immune responses including increasing immune cell infiltration and activity of effector cells while simultaneously reducing suppressive immune cell levels and function. Owing to their respective modes of actions, there is the potential for synergistic effects between CLN-619 and chemotherapeutic agents.

Platinum agents such as carboplatin and cisplatin have been shown to augment MICA/B expression, as exemplified in a small study of carboplatin treated ovarian cancer patients and preclinically with cisplatin treated NSCLC cell lines (Capellero 2020; Okita 2019). These agents have also been shown to positively modulate the immune response in preclinical models and in some instances, clinically. In preclinical models, cisplatin treatment resulted in the recruitment/proliferation of effector cells; upregulation of the lytic activity of cytotoxic effectors; and decreases Tregs in the TME (Gameiro 2012; de Biasi 2014). While no impact to NK cells was seen when treated with carboplatin, treatment of ovarian cancer patients with carboplatin resulted in increases CD8+ T cell's capacity to produce IFNγ in ex vivo studies (Wu 2009).

Taxane chemotherapy such as docetaxel has been shown to upregulate MICA/B expression and the NKG2D-MICA pathway was demonstrated to be important for NK cell lysis mediated by docetaxel in preclinical models (Acebes-Huerta, 2015). These agents have also been demonstrated to positively impact the immune function in preclinical models and patients. In a preclinical model and in breast cancer patient samples, docetaxel treatment was shown to increase NKG2D on NK cells and in the case of the preclinical model, docetaxel mediated increased responsiveness to trastuzumab via NKG2D (Di Modica 2016). In NSCLC patients treated with docetaxel, increased infiltration of CD8+ T cells to the tumor was observed (Gao 2019). Lastly, paclitaxel was demonstrated to reprogram tumor associated macrophages to a more M1 or immunogenic phenotype both preclinically and in patient samples (Wanderley 2018)

The folate antimetabolite, pemetrexed, while not shown to induce MICA/B expression, has been shown to have a positive impact on T cell activity (Okimoto 2020; Schaer 2019). In preclinical studies, pemetrexed increased T-cell activation in mouse tumors in vivo, robustly induced immunogenic cell death in mouse tumor cells and enhanced T-cell activation in vitro (Schaer 2019). Further it was shown to be capable of inducing PD-L1 expression in NSCLC cells and when combined with PD-1 blockade, resulted in significantly enhanced anti-tumor activity in a preclinical model (Schaer 2019).

In addition to the above data highlighting the role of chemotherapy in positively modulating both the MICA-NKG2D pathway and immune effector activity, chemotherapy with immunotherapy has been tested extensively with anti-PD (L) 1 therapies in multiple tumor types. Pembrolizumab combined with chemotherapy, including platinums and taxanes, is approved in multiple tumor types such as NSCLC, endometrial cancer, breast, and gastric cancers (pembro uspi 2021). Dostarlimab is also approved in combination with platinum-based chemotherapy for 1st line treatment of patients with mismatch repair deficient (dMMR) advanced endometrial cancer (N Engl J Med 2023; 388:2145-58. DOI: 10.1056/NEJMoa2216334). While anti PD(L)1 combination with chemotherapy has been successful in some solid tumors, they have not demonstrated efficacy in other solid tumors such as epithelial ovarian cancer or targetable-driven NSCLC such as those with EGFRm or ALK rearrangement.

Cytotoxic chemotherapy can produce immunomodulatory effects, such as disruption of immunosuppressive pathways and enhanced cytotoxic T-cell response. Thus, the combination of chemotherapy and immunotherapy may have synergistic effects in the tumor microenvironment. Clinical benefits, including improved survival, have been reported with this combination in several cancer types. Given the mechanism of actions of individual cytotoxic agents inducing cellular stress and MICA/B expression, there is strong clinical rationale that combining chemotherapy with CLN 619 could be synergistic and will be explored in this study.

### Rationale for Starting Dose and Schedule

The FIH dose and dosing regimen for CLN-619 was calculated utilizing allometric scaling to estimate human PK and expected receptor occupancy (RO), in addition to in vivo and in vitro safety and pharmacology data. Using this methodology, a starting dose of 0.1 mg/kg with a Q3W dosing schedule is proposed for the CLN-619 clinical trial. Based on the totality of the available toxicology and pharmacology data, the starting dose is anticipated to be safe yet within the lower range of pharmacological activity of CLN-619.

Human PK parameters were determined based on allometric scaling from cynomolgus monkey PK data. The expected clearance in humans is calculated to be 0.19 L/day. The projected terminal half-life in humans is 26.9 days, which is typical for a monoclonal antibody and justifies selection of a Q3W dosing schedule.

The NOAEL (101.4 mg/kg/week) in the one-month repeat dose monkey toxicity study supports 0.1 mg/kg as a safe starting dose. At the proposed starting dose of 0.1 mg/kg, after a single dose of CLN-619, the projected area under the concentration curve (AUC0-168) and Cmax are 775-fold and 1,020-fold lower, respectively, than the AUC0-168 and Cmax at the highest non-severely toxic dose (equivalent to the NOAEL) in cynomolgus monkeys, providing a wide safety margin.

The lack of findings observed after administration of CLN-619 to monkeys is consistent with target biology. Notably, expression of MICA/MICB in normal tissues is highly restricted. MICA/MICB expression is induced under stressed conditions (e.g., infection, radiation, malignancy), which is a normal physiological process that triggers the elimination of MICA/MICB expressing cells by immune cells. In healthy monkeys, high levels of normal tissue expression of MICA and MICB would not be expected, and therefore the impact of CLN-619 treatment on normal animals is expected to be minimal.

A modeling approach was used to determine the predicted RO of CLN-619 for MICA and MICB utilizing in vitro association rate constants and equilibrium dissociation constants. The total target occupancy of MICA/MICB is predicted to be approximately 30% at the starting dose for CLN 619. The projected RO of 30% is considered reasonable based on the intended administration of CLN 619 to late-stage cancer patients where the objective is to start at a pharmacologically active dose (PAD) yet taking into account patient safety.

Consideration was also given to the in vivo and in vitro pharmacology data in determining the starting dose for CLN-619. The proposed starting dose for CLN-619 is expected to be within the lower range of pharmacological activity based on both in vivo and in vitro pharmacology data. At the proposed 0.1 mg/kg starting dose, administered as a 1-hour infusion, the anticipated Cavg in human is about 5-fold lower than the measured Cavg attained in mice at the efficacious dose in an in vivo xenograft mouse study. For in vitro functional studies that measured CLN-619 target modulation and Fc effector functions in tumor cell lines, the EC50 values ranged from 0.045 to 0.1 μg/mL. However, the relevance of the in vitro data to FIH dose determination is presented with caution, given that tumor exposure levels are predicted to be 10-fold lower than levels in circulation, resulting in an overestimation of drug potency in the tumor. In addition, while in vitro functional assays are technically sound for demonstration of mechanism driven activity, the Sponsor believes that these assessments are of limited value, as the in vitro assays are closed, static systems and are not expected to be representative of the open, dynamic in vivo situation, where CLN-619 is continuously cleared over time.

In vitro cytokine release assays further support the expectation that the starting dose will be both pharmacologically active and safe. CLN-619 treatment with human PBMC (6 donors) failed to elicit a cytokine/chemokine response above background levels in either soluble or wet-bound formats at all tested concentrations. In the context of a co-culture system of PBMC with a MICA/MICB-expressing cell line, CLN-619 induced release of a limited set of cytokines considered to be on-target pharmacodynamic effects, including MIP1α, TNFα and IL-8. In the co culture system, levels of TNFα were 16 to 210-fold lower than the positive control across the 6 donors at the concentration level closest to predicted Cmax in human. In totality, based on the in vitro human cytokine release data and the lack of cytokine release in the one-month pivotal in vivo monkey toxicity study, there is a low risk of cytokine release at the clinical starting dose of CLN 619.

Based on this data, the starting dose of 0.1 mg/kg with a Q3W dosing schedule was selected and was anticipated to be safe yet within the lower range of pharmacological activity of CLN-619.

Preliminary analysis of CLN-619 PK data collected for dose levels ranging from 0.1 mg/kg to 10 mg/kg have indicated that, at least for doses ranging from 1 mg/kg to 10 mg/kg, the increase in exposure is proportional to the dose. Therefore, at these dose levels and higher, the PK of CLN-619 appears to be linear such that the increase in total exposure will be similar to the increase in the total dose. At the higher dose levels, the mean half-life has ranged as high as 19 days indicating that achievement of steady-state exposures may not occur until 60 to 90 days or approximately 3 to 6 cycles of CLN-619.

In order to more rapidly achieve steady-state exposures but limit the maximum serum concentrations of CLN-619, a loading dose strategy may be employed in the first cycle. The planned loading dose regimen will administer three (3) 10 mg/kg doses of CLN-619 on a weekly schedule in the first cycle. On Day 1 of the second cycle, an additional 10 mg/kg dose will be administered and from the second cycle on, CLN-619 will be administered on a Q3W schedule. As described above, the total increase in the dose will be directly proportional to the total increase in exposure. Therefore, in the first cycle, there will be an approximately 3-fold increase in the overall exposure (AUC) compared to the first cycle on the 10 mg/kg Q3W schedule. However, during this period, overall exposures are not expected to greatly exceed steady-state exposures observed at the 10 mg/kg Q3W schedule which was shown to be well tolerated. With the loading dose, steady-state exposures are expected to be achieved by the end of the first cycle which may provide a higher likelihood of achieving a rapid response.

Given that the loading dose is not expected to achieve systemic exposures higher than previously observed at steady-state on the current 10 mg/kg Q3W, this increase in dose is anticipated to be safe.

### Safety and Risk/Benefit Assessment

As of 22 Jun. 2023, 61 patients have been exposed to doses of CLN-619 at 0.1, 0.3, 1, 3, 6, and 10 mg/kg. Forty-three have been exposed to monotherapy of CLN-619 with 12 patients receiving the 10 mg/kg dose. Eighteen patients have been exposed to CLN-619 at 1, 3, 6, and 10 mg/kg in combination with pembrolizumab with 6 receiving the 10 mg/kg dose. Cumulatively, 23 treatment emergent SAEs were reported following administration of CLN-619 (in monotherapy and in combination with pembrolizumab). A serious adverse event of infusion related reaction (IRR) with laryngeal edema was reported in a single patient that was related to CLN-619, in the absence of mandated steroid premedication. There was 1 non-serious grade 3 rash reported in monotherapy (Module A). There were no serious adverse reactions to CLN-619 reported within the combination therapy (Module B), but 1 SAE of acute kidney injury (AKI) considered possibly related and expected to pembrolizumab (not related to CLN-619). No Aes met protocol defined dose-limiting toxicity (DLT) criteria, and there had been no Grade ≥4 TEAEs. The most common treatment emergent adverse events (TEAEs) to date in ≥5% of patients treated with monotherapy CLN-619 include IRR (23.3%), abdominal pain (18.6%) and fatigue (18.6%), and pyrexia (18.6%). The most common treatment emergent adverse events to date in ≥5% of patients treated with CLN-619 in combination with pembrolizumab include constipation (22.2%), anemia (16.7%), back pain (16.7%), blood creatinine increased (16.7%), fatigue (16.7%), and nausea (16.7%). The most updated version of Investigator's Brochure (IB) provides more detailed safety information.

The estimated frequency of IRR with first dose was 21.3% (13 occurrences in 61 dosed patients). The estimated frequency at any dose was 6.3% (15 occurrences in 235 administrations of CLN-619). The majority of IRRs (13) occurred during or immediately after the first dose; in 1 case the IRR occurred after sixth dose; and 1 patient had IRR after both the first and second dose. There was 1 event that occurred in a patient who received combination treatment with pembrolizumab. The number of patients exposed to the combination therapy is still limited (18) therefore it is difficult to assess if this addition alters the risk of IRR. While all patients recovered without sequalae, IRRs can potentially be severe in some patients.

The infusion time of 60 minutes and IV administration was initially selected for this study and is supported by the nonclinical toxicology studies in which CLN-619 was administered as an IV slow bolus injection over 5 minutes once weekly. Based on clinical experience in the dose escalation cohorts, the infusion time has been extended to 120 minutes for the first 1-2 infusions with the intent to reduce the frequency and severity of IRR.

CLN-619-001 is the first-in-human clinical trial of CLN-619 administered alone and in combination with pembrolizumab (anti-PD-1 antibody) and will be performed in patients with locally advanced or metastatic solid tumors. Patients in the Module B cohorts will be required to have tumor types listed in the current prescribing information for pembrolizumab. Limitations on prior treatment history are described in the inclusion criteria. Unless otherwise specified in the inclusion criteria, patients will be required to have received approved standard therapy that is available to them, declined this therapy, or demonstrated a contraindication or intolerance to standard therapy. CLN-619-001 utilizes a modular study design, with Module A designed to investigate the safety, tolerability, and preliminary anti-tumor activity of CLN-619 monotherapy, while a bifurcated design will support Module B to investigate CLN-619 in combination with pembrolizumab (anti-PD-1 antibody) (see FIG. 23B). Module A Monotherapy Dose Escalation will assess the safety of multiple escalating dose levels of CLN-619, to define the maximum tolerated dose (MTD) and recommended phase 2 dose (RP2D) of CLN-619 monotherapy. Assuming acceptable safety and tolerability for CLN-619 monotherapy in dose escalation, the Module A Cohort Expansion will initiate, with enrollment of tumor-specific cohorts of patients with non-small cell lung cancer (NSCLC), cervical cancer and endometrial cancer. Up to three additional cohorts of patients with specific tumor types may be enrolled in the Module A Cohort Expansion based on evidence for clinical benefit and/or any observed relationships between blood and tumor-based biomarker data and objective response observed during the study or other applicable emerging data. These cohorts will further evaluate the PK/pharmacodynamics (PD), safety and initial anti-tumor activity of CLN-619 monotherapy.

A bifurcated trial design will also support initiation of the Module B Combination Therapy Dose Escalation Cohort in patients with advanced solid tumors. This cohort will investigate the safety, tolerability and initial anti-tumor activity of CLN-619 in combination with pembrolizumab using a 3+3 design. Once the safety of the 3 mg/kg monotherapy dose has been established, a Module B Combination Therapy Dose Escalation Cohort may be initiated at a CLN-619 dose of 1 mg/kg. If the monotherapy CLN-619 MTD is below 3 mg/kg then Module B Combination Therapy Dose Escalation Cohort may be initiated at one dose level lower than the MTD. Pembrolizumab (200 mg) will be administered immediately prior to CLN-619. A standard 3+3 dose escalation scheme will be followed. Dose levels of 1.0, 3.0, 6.0, and10 mg/kg are planned.

Subsequent to demonstration of acceptable safety and tolerability for the combination of CLN-619 with pembrolizumab, the Module B Cohort Expansion will evaluate the preliminary safety and anti-tumor activity of CLN-619 combination with pembrolizumab in patients with NSCLC and endometrial cancer. Up to three additional disease-specific expansion cohorts may be enrolled in the Module B Cohort Expansion based on evidence for clinical benefit and/or any observed relationships between blood and tumor-based biomarker data, objective response or stable disease observed or other applicable emerging data. Monotherapy dose escalation cohorts and combination therapy dose escalation cohorts will enroll patients in a conservative, step-wise fashion guided by standard decision rules and with close oversight.

Module C will explore the safety, tolerability, and preliminary efficacy in pre-defined tumor types in combination standard of care chemotherapy. Combination dose expansion cohorts will enroll patients in a conservative, step-wise fashion guided by standard decision rules and with close oversight.

With the addition of Module D, the Sponsor will investigate if the loading dose of weekly dosing for cycle 1 can achieve steady-state exposures by the end of the first cycle which may provide a higher likelihood of achieving a rapid response. Given that the loading dose is not expected to achieve systemic exposures higher than previously observed at steady-state on the current 10 mg/kg Q3W which was shown to be well tolerated, this increase in dose is anticipated to be safe. Taken together, this study offers a reasonable risk: benefit assessment for advanced cancer patients with few therapeutic options.

### Module A Monotherapy Dose Escalation

Cohorts of patients with advanced select solid tumors will be treated with ascending doses of CLN-619 monotherapy, initially using a single-patient accelerated titration design. Transition to a standard 3+3 dose escalation scheme will occur once the first patient enrolled at a given dose level experiences a Grade ≥2 CLN-619 related (defined as possibly or definitely related) adverse event (AE) during the initial three-week treatment cycle or at the 3.0 mg/kg dose level, whichever comes first. Planned dose levels are shown in Table 3 below.

TABLE 3
Module A Monotherapy Doses During Escalation

| Cohort | CLN-619 Dose | |
|---|---|---|
| Dose Level 1 | 0.1 | mg/kg |
| Dose Level 2 | 0.3 | mg/kg |
| Dose Level 3 | 1.0 | mg/kg |
| Dose Level 4 | 3.0 | mg/kg |
| Dose Level 5 | 6.0 | mg/kg |
| Dose Level 6 | 10 | mg/kg |

Data obtained during the Module A Dose Escalation will be used to identify a MTD (or a MAD if no MTD is defined) for CLN-619 monotherapy. Alternatively, a MBED may be designated at a dose below the MTD based on a review of PK/PD, safety and efficacy data, and may also guide dose selection (i.e., the RP2D) for the Module A Cohort Expansion.

### Module A Monotherapy Dose Escalation Dose-Level Cohort Extension

The purpose of extending enrollment in these dose level cohorts is to further characterize safety, PK, PD, biomarker, and clinical activity of CLN-619, as well as characterize the clinical and biological activity of dose levels selected for extension.

Up to 10 patients with advanced solid tumors, inclusive of those enrolled in the original dose level cohort, were planned for enrollment in each cohort extension for dose levels selected. Collectively, 45 patients were enrolled in the 7 cohorts.

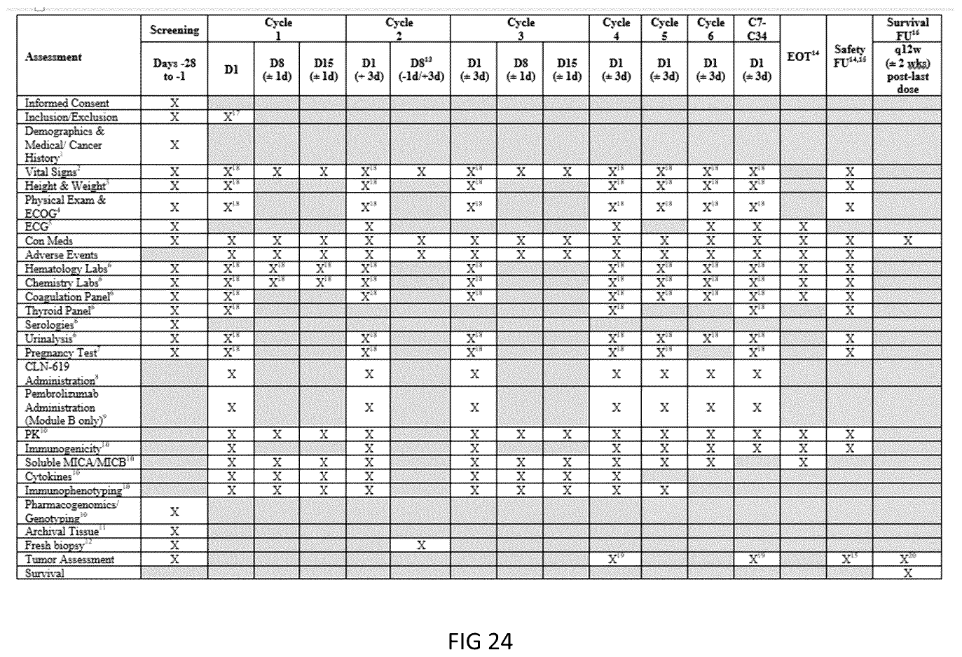

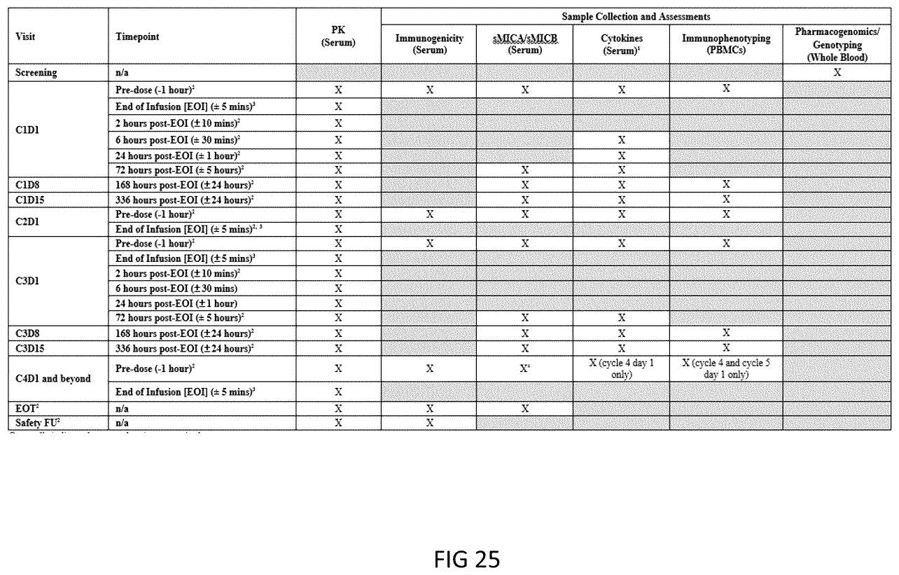

Study procedures in the cohort extensions are identical to those in the original cohort as described in the Schedule of Events (FIG. 24) and Sampling Schedule (FIG. 25) with exception of two mandatory fresh biopsies collected at baseline (screening) and following the first cycle of dosing (i.e., C2D8-1 day/+3 days).

### Module A Monotherapy Cohort Expansion

Upon selecting a RP2D of CLN-619 monotherapy, the Module A Monotherapy Cohort Expansion will be initiated. This module will investigate the safety and initial anti-tumor activity of CLN-619 monotherapy, enrolling to tumor-specific expansion cohorts:
Expansion A1: CLN-619 monotherapy in patients with non-small cell lung cancer (NSCLC) (N=16 patients).
Expansion A2: CLN-619 monotherapy in patients with cervical cancer (N=16 patients).
Expansion A3: CLN-619 monotherapy in patients with endometrial cancer (N=10 patients) at dose level #1
Expansion A4: CLN-619 monotherapy in patients with endometrial cancer (N=10 patients) at dose level #2
Up to 3 additional disease-specific expansion cohorts may be declared based on evidence for clinical benefit (objective response or stable disease extending through at least 6 cycles of treatment) in that tumor type during the dose escalation phase of the study. Alternatively, the relationships between blood and tumor-based biomarker data and objective response may be considered in triggering these additional tumor specific expansion cohorts.
Expansions A1 and A2: if 2 of 16 patients experience clinical benefit (defined as objective response or stable disease extending through at least 6 cycles of treatment), that cohort may be further expanded, enrolling up to a total of 40 patients to further characterize the preliminary anti-tumor activity of CLN-619 monotherapy in that population.
Expansions A3 and A4: Each cohort will initially expand to 10 patients, inclusive of those dosed at the respective dose level during dose escalation. If at least 1 objective response is observed among the first 10 patients treated, additional patients may be enrolled up to 40 total patients with endometrial cancer (20 at each dose level, inclusive of those dosed during the dose escalation phase).
Additional disease-specific expansion cohorts: each cohort will initially expand to 10 patients. If there is evidence of clinical benefit (objective response or stable disease extending through at least 6 cycles of treatment) in at least 1 of the first 10 patients treated, additional patients may be enrolled up to 40 total patients with a defined tumor type (inclusive of those dosed during the dose escalation phase).

### Module B Combination Therapy Dose Escalation

Module B Combination Therapy Dose Escalation will be initiated to investigate the safety, tolerability, and initial anti-tumor activity of CLN-619 in combination with pembrolizumab. These cohorts will enroll patients with tumor types described in the current prescribing information for pembrolizumab. Cohorts may be enrolled in parallel with the Module A Monotherapy Expansion Cohorts with initiation of the Dose Level 1 (1 mg/kg) upon demonstration of safety of the 3 mg/kg monotherapy dose level. If a lower dose is determined to be the MTD in the Module A monotherapy dose escalation arm, then dose levels below the MTD will be investigated in combination with pembrolizumab.

Planned dose levels are shown in Table 4 below.

TABLE 4
Module B Combination Therapy Doses During Escalation

| Cohort | CLN-619 Dose | | Pembrolizumab Dose¹ |
|---|---|---|---|
| Dose Level −2 | 0.1 | mg/kg | 200 mg |
| Dose Level −1 | 0.3 | mg/kg | 200 mg |
| Dose Level 1 | 1.0 | mg/kg | 200 mg |
| (starting dose)² | | | |
| Dose Level 2 | 3.0 | mg/kg | 200 mg |
| Dose Level 3 | 6.0 | mg/kg | 200 mg |
| Dose Level 4 | 10 | mg/kg | 200 mg |

¹Pembrolizumab to be administered immediately prior CLN-619
²Module B Combination Therapy Dose Level 1 (1 mg/kg) will be initiated once safety is established by the SRC in Module A, CLN-619 dose of 3.0 mg/kg. Dose levels −1 and −2 may be assessed as described in the decision rules.

If no DLTs are observed during the first three-week cycle for the initial three patients at Dose Level 1, CLN-619 will be escalated. In the event of DLTs as noted in the decision rules, de-escalation may proceed at dose levels listed in Table.

### Module B Combination Therapy Cohort Expansion

Upon demonstrating acceptable safety and tolerability for CLN-619 in combination with pembrolizumab, the Module B Cohort Expansion will be initiated. This module will investigate the safety and initial anti-tumor activity of CLN-619 in combination with pembrolizumab by enrolling patients with NSCLC, endometrial cancer, and up to three additional tumor types. Module B Cohort Expansion can be initiated only after six patients have been enrolled at the same dose level and the safety of the dose level has been declared based upon the 3+3 design decision rules.
Expansion B1: CLN-619+pembrolizumab in patients with NSCLC (N=16)
Expansion B2: CLN-619+pembrolizumab in patients with endometrial cancer (N=16)
Up to 3 additional disease-specific cohorts may be declared based on evidence for clinical benefit (objective response or stable disease extending through at least 6 cycles of treatment) in that tumor type during the dose escalation phase of the study. Alternatively, the relationships between blood and tumor-based biomarker data and objective response may be considered in triggering these additional tumor specific expansion cohorts.
Expansion B1 and additional disease-specific expansion cohorts: if 2 of 16 patients experience clinical benefit (defined as objective response or stable disease extending through at least 6 cycles of treatment), that cohort may be further expanded, enrolling up to a total of 40 patients (inclusive of those treated in the dose escalation phase of the study) to further characterize the safety and preliminary anti-tumor activity of CLN-619 monotherapy in that population.
Expansion B2: patients will be alternately assigned to receive one of two dose levels of CLN-619 in combination with pembrolizumab. If 2 of 16 patients experience clinical benefit (defined as objective response or stable disease extending through at least 6 cycles of treatment), the cohort may further enroll up to a total of 40 patients.

### Module C CLN-619+Chemotherapy Combination Therapy, Escalation and Expansion

Module C Dose Escalation: Each of the chemotherapy combination cohorts will be investigated at the 3 mg/kg and 10 mg/kg doses. Patients will be enrolled at 3 mg/kg CLN-619 in combination with standard doses of chemotherapy based on a 3+3 design. For each tumor type, CLN-619+chemotherapy combination will start at 3 mg/kg (n=3-6) and escalate to 10 mg/kg (n=3-6) once safety is confirmed. If DLT occurs at 10 mg/kg, a lower dose will be chosen, and the safety process will be repeated. Safety data for each dose level tested in combination with chemotherapy for each tumor type will be reviewed to determine the optimal dose to enroll the remainder of the patients in Dose Expansion, up to a total of 40.

Module C Disease Specific Dose Expansion: Initially 16 patients will be enrolled in each cohort. If 2 of 16 patients experience clinical benefit (defined as objective response or stable disease extending through at least 6 cycles of treatment), the cohort may be further expanded, enrolling up to a total of 40 patients to further characterize the safety and preliminary anti-tumor activity of CLN-619 and chemotherapy combination in that population.
Cohort C1: CLN 619+carboplatin+pemetrexed in recurrent EGFRm NSCLC
Cohort C2: CLN 619+carboplatin+paclitaxel in recurrent endometrial cancer recurrent
Cohort C3: CLN 619+paclitaxel in platinum-resistant epithelial ovarian cancer
Each treatment cycle will be every 21 days or 3 weeks (q21d or q3w)

TABLE 5
C1: Recurrent EGFRm NSCLC

| Cohort | CLN-619 Dose ** | | Chemotherapy Dose |
|---|---|---|---|
| Dose Level 1 | 3.0 | mg/kg | Carboplatin AUC 5 + pemetrexed |
| | | | 500 mg/m² |
| Dose Level 2 | 10 | mg/kg | Carboplatin AUC 5 + pemetrexed |
| | | | 500 mg/m² |

TABLE 6
C2: Recurrent endometrial cancer

| Cohort | CLN-619 Dose ** | | Chemotherapy Dose |
|---|---|---|---|
| Dose Level 1 | 3.0 | mg/kg | Carboplatin AUC 5-6 + paclitaxel |
| | | | 175 mg/mg² q3w |
| Dose Level 2 | 10 | mg/kg | Carboplatin AUC 5-6 + paclitaxel |

175 mg/mg² q3w

TABLE 7
C3: Platinum-resistant epithelial ovarian cancer

| Cohort | CLN-619 Dose** | | Chemotherapy Dose |
|---|---|---|---|
| Dose Level 1 | 3.0 | mg/kg | Paclitaxel 60-80 mg/m² q1w* |
| Dose Level 2 | 10 | mg/kg | Paclitaxel 60-80 mg/m² q1w* |

*Weekly doses can be held per the investigator's discretion per usual practice.
**If DLT occurs at 10 mg/kg, a lower dose will be chosen, and the safety process will be repeated.

### Module D Loading Dose Cohort

This module will investigate the safety and anti-tumor activity of CLN-619 monotherapy. A weekly loading dose of 10 mg/kg will be administered in Cycle 1 (QWx3). Then 10 mg/kg Q3W in Cycle 2 and subsequent cycles (follows the regular dosing schedule).

### Selection of Study Population

### Number of Planned Patients

Full enrollment of all modules will be up to approximately 640 patients, not counting patients who will need to be replaced. For Module A monotherapy dose escalation, 45 patients with advanced solid tumors have been enrolled. Up to 240 patients with specific tumor types will be enrolled in Module A monotherapy cohort expansions. If initiated, 25 patients have been enrolled in the Module B combination therapy dose escalation cohort, and up to 200 patients will be enrolled in the Module B and up to 120 patients will be enrolled in Module C CLN-619+Chemotherapy Combination Therapy, Escalation and Expansion Cohort. For Module D monotherapy loading dose cohort, up to 10 patients will be enrolled.

### Inclusion Criteria

Patients must fulfill all the following inclusion criteria to be eligible for participation in the study.
1) Males or females aged ≥18 years.
2) Willing and able to give written informed consent and adhere to protocol requirements; written informed consent and any locally required authorization must be obtained from the patient prior to performing any protocol-related procedures, including screening evaluations.
3) Module A Monotherapy Dose Escalation Cohort and Module B Combination Therapy Dose Escalation Cohorts: Histologically or cytologically-confirmed metastatic or locally advanced, unresectable solid tumors. For Module B, tumor type is listed as an approved indication per the current prescribing information for pembrolizumab.
4) Module A Cohort Expansions:
   a) Expansion A1: Histologically or cytologically-confirmed metastatic or locally-advanced, unresectable NSCLC;
   b) Expansion A2: Histologically or cytologically-confirmed metastatic or locally-advanced, unresectable cervical cancer.
   c) Expansion A3 and A4: Histologically or cytologically-confirmed metastatic or locally-advanced, unresectable endometrial cancer.
   d) Eligibility for disease-specific expansion cohorts may be further refined by histologic subtype, molecular features, or exposure to prior therapy based on clinical, pharmacodynamic, or biomarker data emerging from the study.
5) Module B Cohort Expansions:
   a) Expansion B1: Histologically or cytologically-confirmed metastatic locally-advanced, unresectable NSCLC.
   b) Expansion B2: Histologically or cytologically-confirmed metastatic or locally-advanced, unresectable endometrial cancer.
   c) Eligibility for disease-specific expansion cohorts may be further refined by histologic subtype, molecular features, or exposure to prior therapy based on clinical, pharmacodynamic, or biomarker data emerging from the study.
6) Module C CLN-619+Chemotherapy Combination Therapy, Escalation and Expansion Cohort
   a) Expansion C1: Histologically or cytologically confirmed recurrent NSCLC.
   b) Expansion C2: Histologically or cytologically confirmed recurrent endometrial cancer.
   c) Expansion C3: Histologically or cytologically-confirmed recurrent, platinum-resistant epithelial ovarian cancer.
   d) Eligibility for disease-specific expansion cohorts may be further refined by histologic subtype, molecular features, or exposure to prior therapy based on clinical, pharmacodynamic, or biomarker data emerging from the study.
7) Module D Monotherapy Loading Dose 10 mg Cohort: Recurrent epithelial ovarian, breast, and gastrointestinal (esophageal, gastric, colorectal) cancer patients.
8) Prior treatment history as follows:
   a) Patients should have received any other approved standard therapy that is available to the patient, unless this therapy is contraindicated, intolerable to the patient, or is declined by the patient. In the case of a patient declining such therapy, documentation that the patient has been informed and declined should be documented in the medical record.
9) At baseline, patients are required to have one or more measurable lesions that meet RECIST v1.1 and meet the following conditions:
   a) A non-lymph node lesion that has a longest unidimensional measurement of ≥10 mm or a lymph node lesion that has a shortest unidimensional measurement of ≥15 mm;
   b) Lesions that have received previous local treatment, such as radiotherapy or ablation, can also be used as measurable target lesions if progression has been confirmed according to RECIST v1.1 prior to enrollment, and the longest unidimensional measurement is ≥10 mm.
10) Performance status of 0 or 1 based on the Eastern Cooperative Oncology Group (ECOG) performance scale.
11) Estimated life expectancy of 12 weeks or greater.
12) Prior palliative radiotherapy must have been completed 14 days prior to dosing on C1D1.
13) Toxicities related to prior study therapy should have resolved to Grade 1 or less according to the National Cancer Institute (NCI) Common Terminology Criteria for Adverse Events (CTCAE) v5.0, except for alopecia. Peripheral neuropathy should be clinically stable or improving and be Grade 2 or less in severity. Patients with chronic but stable Grade 2 toxicities may be allowed to enroll after agreement between the Investigator and Sponsor.
14) Have adequate liver and kidney function and hematological parameters within a normal range as defined by:
   a) Total bilirubin≤1.5× ULN. This does not apply for patients with confirmed Gilbert's Syndrome, for whom total bilirubin must be less than 3.0 mg/dL with a conjugated bilirubin less than 0.5 mg/dL;
   b) Aspartate aminotransferase (AST) and alanine aminotransferase (ALT)≤2.5× ULN or ≤5× ULN for patients with liver metastases;
   c) Creatinine clearance (CrCl) ≥45 mL/min as measured or estimated using Cockcroft-Gault formula;
   d) Hemoglobin ≥8 g/dL without blood transfusions for at least two weeks prior to dosing on C1D1;
   e) Absolute neutrophil count ≥1500 cells/mm³ without growth factor support, three days for filgrastim, 14 days for pegfilgrastim;
   f) Platelet count ≥75,000 cells/mm³.
15) Patients in the Module A and Module B dose escalation cohorts and Module D must have archival tissue available for biomarker analysis. The sample should preferably be from a sample obtained after the most recent therapy. A fresh biopsy is required if archival tissue (e.g., all tumor blocks are exhausted) is unavailable. If a biopsy cannot be performed with acceptable clinical risk in the judgment of the Investigator the Sponsor's Medical Monitor must be contacted to approve enrollment.
   a) Patients participating in the Module A and Module D dose-level cohort extension(s) must agree to provide a fresh biopsy at screening and an on-treatment biopsy at Cycle 2, Day 8.
   b) Patients in the expansion cohorts for Module A, B and C must agree to provide a fresh pretreatment biopsy and an on-treatment biopsy at Cycle 2, Day 8. If a biopsy cannot be performed with acceptable clinical risk in the judgment of the Investigator to allow the required biopsies, the Sponsor's Medical Monitor must be contacted to approve enrollment. Paired biopsy is recommended if clinically feasible for Module A1 and B1 NSCLC Cohorts.

### Exclusion Criteria

Patients will be excluded if they fulfil any of the following exclusion criteria:
1) Currently participating/previously participated in an interventional study and received an investigational drug within 28 days (or five half-lives, whichever is longer) of dosing on C1D1.
2) Patients with concomitant second malignancies (except adequately treated non-melanomatous skin cancers, ductal carcinoma in situ, superficial bladder cancer, prostate cancer or in situ cervical cancer) are excluded unless in complete remission three years prior to study entry, and no additional therapy is required or anticipated to be required during study participation.
3) Patients with any active autoimmune disease or a history of known or suspected autoimmune disease, or history of a syndrome that requires systemic corticosteroids or immunosuppressive medications, except for patients with vitiligo, resolved childhood asthma/atopy or autoimmune thyroid disorders on stable thyroid hormone supplementation.
4) A serious uncontrolled medical disorder that would impair the ability of the patient to receive protocol therapy or whose control may be jeopardized by the complications of this therapy. These criteria include, but are not limited to the following:
   a) Uncontrolled airway hyper-reactivity;
   b) Type 1 diabetes mellitus. Type 2 diabetes mellitus patients are allowed if they are under stable glycemic control as per Investigator assessment;
   c) Uncontrolled, clinically significant pulmonary disease;
   d) Requirement for supplemental oxygen to maintain a pulse ox>93%;
   e) Symptomatic congestive heart failure as per Investigator assessment or documented cardiac ejection fraction less than 45%;
   f) Ejection fraction <45% in patients with prior history of treatment with anthracycline chemotherapy or with a prior history of cardiac ventricular dysfunction. Patients with prior history of ventricular dysfunction or anthracycline therapy are required to have an echocardiogram for assessment of baseline cardiac function;
   g) History of unstable angina or myocardial infarction within six months of dosing on C1D1;
   h) Unstable cardiac arrhythmia;
   i) History of ventricular arrhythmia;
   j) Uncontrolled hypertension: patients with sustained systolic blood pressure readings greater than 150 or diastolic blood pressure greater than 100 should have documentation by treating physician that the finding is not consistent with uncontrolled hypertension;
   k) History of stroke or cerebral hemorrhage within one year of dosing on C1D1;
   l) Poorly controlled seizure disorder;
   m) Active diverticulitis within one year prior to dosing on C1D1;
   n) Recent major surgery within three months of dosing on C1D1 or major surgery with unresolved complications that could interfere with study treatment.
5) Treatment with systemic antiviral, antibacterial or antifungal agents for acute infection within ≤7 days of dosing on C1D1.
6) Has a history of, or a positive test for, HIV½ primary immunodeficiency disease such as Human Immunodeficiency Virus (HIV).
7) Diagnosed with hepatitis B (with positive testing for either hepatitis B surface antigen [HbsAg] or hepatitis B core Ab) or hepatitis C virus (HCV) infection (with positive testing for HCV antibody and/or HCV ribonucleic acid [RNA] in serum) under any of the following conditions:
   a) Active disease for hepatitis B or hepatitis C and received antiretroviral therapy within 4 weeks.
   b) Blood hepatitis B deoxyribonucleic acid (DNA) or HCV RNA are detectable.
8) Prior organ allograft or allogeneic hematopoietic transplantation.
9) History of the following events in conjunction with prior treatment with checkpoint inhibitor immunotherapy: Grade 3 or greater neurotoxicity, ocular toxicity, pneumonitis, myocarditis, or colitis; liver dysfunction meeting the laboratory criteria for Hy's Law.
10) Symptomatic uncontrolled brain metastases, known or suspected leptomeningeal metastases and/or carcinomatous meningitis. Patients with brain metastases identified at Screening may be rescreened after they have been appropriately treated (e.g., Surgery and/or radiotherapy). Patients with treated brain metastases should be neurologically stable for 28 days post completion of treatment and prior to enrollment, and on a stable regimen of steroid dosing (prednisone <10 mg or the equivalent) for 14 days prior to dosing on C1D1.
11) Treatment with non-oncology vaccines for the control of infectious diseases (i.e., HPV vaccine) within 28 days of C1D1. The inactivated seasonal influenza vaccine can be given to patients before initiation of treatment, and while on study therapy without restriction. Influenza vaccines containing live virus, or other clinically indicated vaccinations for infectious diseases (i.e., pneumovax, varicella) may be permitted, but must be discussed in advance with the Sponsor's Medical Monitor and may require a study drug washout period before and/or after administration of the vaccine. Covid-19 vaccines may be administered according to institutional policy.
12) Active SARS-COV-2 infection including history of positive SARS-COV-2 testing without subsequent documentation of negative test results, patients with results that are pending but not yet known, or patients with suspected active infection based on clinical features. SARS-COV-2 vaccination is permitted on treatment.
13) Has received immunosuppressive medications including but not limited to cellcept, methotrexate, infliximab, anakinra, tocilizumab, cyclosporine or corticosteroids (≥10 mg/day of prednisone or equivalent), within 28 days of dosing on C1D1.
14) Female of child-bearing potential (FOCBP) who is pregnant or breast-feeding, plans to become pregnant within 120 days of last study drug administration, or declines to use an acceptable method to prevent pregnancy during study treatment and for 120 days after the last dose of study drug administration.
a) A female of childbearing potential is defined as:
   i) Not surgically sterile, i.e., bilateral tubal ligation, bilateral oophorectomy, or complete hysterectomy, or;
   ii) Not post-menopausal, defined as amenorrhea for >two years without an alternative medical cause.
   Note: Females with amenorrhea for <two years and who are not surgically sterile i.e., tubal ligation, bilateral oophorectomy, or complete hysterectomy will only be considered not to be of reproductive potential if they have a documented follicle stimulating hormone (FSH) value in the postmenopausal range.
15) Male patient who plans to father a child or donate sperm within 120 days or 5 half-lives of CLN-619, whichever comes later, of last study drug administration, or who has a partner who is a FOCBP, and declines to use acceptable method to prevent pregnancy during study treatment and for 120 days or 5 half-lives of CLN-619, whichever comes later, after the last dose of study drug administration.
16) QT interval corrected for heart rate using Fridericia's formula (QTcF) of ≥500 milliseconds.
17) Patient has history of drug-related anaphylactic reactions to any components of CLN-619 (Module A and Module B patients) or pembrolizumab (Module B patients only). History of Grade 4 anaphylactic reaction to any monoclonal antibody therapy.
18) Known active alcohol or drug abuse.
19) Inability to comply with the protocol and/or not willing or not available for follow-up assessments.
20) Patients who are incapacitated or involuntarily incarcerated.
21) Patients who are unsuitable for participation based on the judgement of the Investigator.
22) Treatment with any of the following:
   a) Systemic anticancer treatment within 14 days prior to the first dose of study drug on C1D1.
   b) Immunotherapy ≤28 days prior to the first dose of study drug on C1D1.
   c) Radiotherapy <28 days and palliative radiation ≤14 days prior to the first dose of study drug on C1D1. If irradiated, lesions must have demonstrated clear-cut progression prior to being eligible for evaluation as target lesions.
   d) Major surgery (excluding placement of vascular access) ≤28 days of the first dose of study drug on C1D1.

### Meals and Dietary Restrictions

Patients should maintain a normal diet unless modifications are required to manage an AE such as diarrhea, nausea, or vomiting.

### Criteria for Treatment Discontinuation

Patients that withdraw from treatment should be encouraged to return for the Safety Follow-up visit and enter survival follow-up. If the patient withdraws from both treatment and follow-up, no further evaluations should be performed, and no additional data should be collected.

Reasons for treatment discontinuation include, but are not limited to, the following:
Completion of 34 cycles of therapy;
Disease progression or requirement for anticancer therapy not specified in the protocol;
Inter-current illness, general, or specific changes in the patient's condition where, in the opinion of the Investigator, further treatment is not in the best interest of the patient;
Death
DLT or unmanageable AE related to therapy;
Withdrawal of consent for treatment;
Significant protocol violation or non-compliance with protocol;
Termination of the study by Sponsor;

For patients who meet the criteria for treatment discontinuation but are otherwise felt to be deriving compelling clinical benefit from ongoing treatment with CLN-619, pembrolizumab, or CLN-619 in combination with pembrolizumab, or CLN-619 in combination with chemotherapy, consideration may be given on a case-by-case basis for continuation of study therapy.

### Criteria for Study Discontinuation (EOT)

Patients should be encouraged to complete all study assessments. However, patients may withdraw consent to participate in this study at any time and for any reason without penalty or loss of benefits to which they were otherwise entitled.

Patients will be withdrawn from study (survival follow-up) in the case of:
Completion of follow-up period;
Withdrawal of consent from follow-up;
Patient lost to follow-up;
Death;
Termination of study by Sponsor, Institutional Review Board (IRB)/Ethics Committee (EC), regulatory, or other governing authority.

### Definition of Dose-Limiting Toxicity (DLT)

Throughout the study, including DLT will be defined by the occurrence of study drug-related Aes (possibly or definitely related to treatment) with CLN-619 alone or in combination with pembrolizumab or in combination with chemotherapy. DLT will be defined separately for hematologic, non-hepatic non-hematologic, and hepatic non-hematologic events as outlined below. The severity of all events will be graded by the CTCAE v5.0. For patients who experience Grade 3 or greater adverse events, treatment with study drug(s) should generally be held pending management and resolution of the event, and assessment of the relationship of the event to administration of the study drug(s).

### Hematologic Dose-Limiting Toxicity (DLT):

Grade 4 neutropenia lasting ≥7 days;
Grade 4 febrile neutropenia;
Grade 3 febrile neutropenia lasting >48 hours or associated with objective evidence of clinical infection;
Grade 4 thrombocytopenia or Grade 3 or greater thrombocytopenia associated with clinically significant bleeding.

### Non-Hepatic, Non-Hematologic Dose-Limiting Toxicity (DLT):

Grade 3 or greater non-hematologic, non-hepatic Aes will be considered a DLT with the following exceptions:
Grade 3 or 4 electrolyte abnormalities that are otherwise not associated with clinical complications and respond to medical intervention within 96 hours;
Grade 3 fatigue lasting less than 72 hours;
Grade 3 fever;
Grade 3 headache that responds to medical intervention within 24 hours;
Grade 3 nausea, vomiting, or diarrhea that responds to medical intervention within 72 hours;
Grade 3 or 4 elevations in amylase or lipase in the absence of radiographic or clinical signs and symptoms suggestive of pancreatitis;
Grade 3 hypertension not associated with clinical complications, and that responds to medical intervention within 24 hours;

### Hepatic, Non-Hematologic Dose-Limiting Toxicity (DLT):

### Transaminase Elevations

Grade 3 or greater elevation of AST or ALT >10× ULN, irrespective of duration;
Grade 3 elevation of AST or ALT ≥5× ULN and ≤10× ULN that fails to resolve to Grade 2 or less within seven days, and to Grade 1 or less within 14 days.

### Bilirubin Elevations

Grade 3 or greater elevation of total bilirubin >5× ULN, irrespective of duration;
Grade 3 elevation of bilirubin ≥3× ULN and ≤5× ULN that fails to resolve to Grade 2 or less within seven days, and to Grade 1 or less within 14 days.

Adverse events that meet the criteria for Hy's Law will also be considered as a DLT:
AST or ALT ≥3× ULN, and
Total bilirubin ≥2× ULN in the absence of cholestasis, and
Absence of demonstrable alternative etiology.

Grade 2 events that are persistent, clinically significant and warrant ongoing dose interruption in a patient may also be deemed a DLT on a case-by-case basis.

### Study Stopping Rules

Study accrual will be paused if there is one Grade 5 event or two Grade 4 events that are considered possibly related to CLN-619 therapy. Grade 4 laboratory abnormalities that resolve to Grade 2 or less within 96 hours will not be included.

### Decision Rules for Module A Accelerated Titration Cohorts

The accelerated titration will switch to a standard 3+3 design at the 3.0 mg/kg dose level unless toxicity at a lower dose level requires initiation at a lower dose level.

Upon completion of a single patient accelerated titration cohort, if:
No CLN-619 related Grade ≥2 Aes occur during Cycle 1; dose escalation may proceed to the next accelerated titration cohort.
The patient experiences any CLN-619 related Grade ≥2 Aes during Cycle 1; convert the current cohort to the 3+3 design and enroll an additional two to five patients.
The patient experiences a DLT; convert to the 3+3 design and enroll an additional two to five patients.

### Decision Rules for Module a Dose Escalation 3+3 Cohorts

Upon completion of a 3+3 cohort (a minimum of three, maximum of six patients), if:
0 of 3 patients experience a DLT; dose escalation may proceed to the next cohort.
1 of 3 patients experience a DLT; an additional three patients will be enrolled into the cohort.
≤1 of 6 patients experience a DLT; dose escalation may proceed to the next cohort.
≥2 patients experience a DLT; the MTD has been exceeded, and further enrollment at that dose level will cease.
   Lower-dose cohorts may then be further explored until the MTD has been determined, including intermediate doses.

### Decision Rules for Module B Dose Escalation 3+3 Cohorts

Upon completion of a dose escalation dose level cohort (a minimum of three, maximum of six patients), if:
0 of 3 patients experience a DLT; dose escalation may proceed to the next dose level cohort.
1 of 3 patients experience a DLT; an additional three patients will be enrolled into the current dose level cohort.
≤1 of 6 patients experience a DLT; dose escalation may proceed to the next dose level cohort provided safety of the next ascending dose level of CLN-619 was established by the SRC during the Module A monotherapy Dose Escalation arm of the study.
≥2 patients experience a DLT; the MTD has been exceeded and further enrollment at that dose level will cease.
   A “minus one” cohort at a lower dose may be enrolled, following the 3+3 decision rules noted above.
      If ≥2 patients experience a DLT in the “minus one” cohort, the Sponsor may proceed with a “minus two” cohort at a lower dose following the 3+3 decision rules noted above.
         If ≥2 patients experience a DLT in the “minus two” cohort, the Sponsor will not proceed with initiation of the Module B Cohort Expansions.
            Decision Rules for Module C Dose Escalation (3 mg/Kg and 10 mg/Kg)

To establish safety at dose level 3 mg/kg
0 of 3 patients at the 3 mg/kg dose level experience a DLT; dose escalation may proceed to the 10 mg/kg dose level.
1 of 3 patients at the 3 mg/kg dose level experiences a DLT; an additional three patients will be enrolled.
≤1 of 6 patients experience a DLT; dose escalation may proceed to the 10 mg/kg dose level
≥2 patients experience a DLT at the 3 mg/kg dose level; the MTD has been exceeded and further enrollment at 3 mg/kg will cease.

To establish safety at dose level 10 mg/kg
0 of 3 patients at the 10 mg/kg dose level experience a DLT dose expansion may proceed at 10 mg/kg
1 of 3 patients at the 10 mg/kg dose level experience a DLT; an additional three patients will be enrolled at 10 mg/kg.
≤1 of 6 patients at the 10 mg dose level experience a DLT; dose expansion may proceed at 10 mg/kg.
≥2 patients experience a DLT at 10 mg/kg; the MTD has been exceeded and further enrollment at 10 mg/kg will cease. Data for each dose level that was tested in combination with chemotherapy for each tumor type will be reviewed to determine the dose to enroll the remainder of the patients in Dose Expansion (up to a total of 40).

### Decision Rules for Module D Dose Escalation

To assess the safety of dosing schedule (10 mg/kg, Cycle 1, Q1W and Cycle 2 and beyond, Q3W), initially 3-6 patients will be enrolled. If there are fewer than 2 instances of Dose Limiting Toxicities (DLTs), additional patients (up to 10) may be enrolled to collect additional PK and biomarker data to establish this dosing schedule 0 of 3 patients experience a DLT; additional patients may be enrolled.
1 of 3 patients experience a DLT; an additional three patients will be enrolled.
≤1 of 6 patients experience a DLT; additional patients may be enrolled. ≥2 patients experience a DLT; the MTD has been exceeded and further enrollment at this dosing schedule will cease.

### Dose Expansion Decision Rules

During the study, if >33% of patients are noted to experience Aes meeting the criteria for DLT during the first three-week cycle or if safety findings suggestive of clinically significant cumulative toxicity are observed, the SRC also may be convened on an ad hoc basis to review data from the expansion cohorts and to make recommendations regarding ongoing dosing of patients.

### Determination of Maximum Biologically-Effective Dose

Module A Monotherapy and Module B Combination Therapy Dose Escalation are both designed to identify an MTD, MAD (if no MTD is defined) and/or a MBED of CLN-619 or CLN-619 in combination with pembrolizumab, respectively, and to inform selection of a RP2D for further investigation in the respective Cohort Expansions. During this stage, PK and exploratory biomarker data will be collected and analyzed in addition to safety and efficacy data to assess whether a dose below the MTD or MAD may possess a more favorable clinical profile.

### Timing and Frequency of Dose Administration

All infusions will be administered in a monitored setting with immediate access to trained personnel and adequate equipment and medicines to manage potentially serious or life-threatening reactions. In dose escalation (Module A and B) CLN-619 will be administered on Day 1 of each three-week treatment cycle as a 60-minute IV infusion (+10/−5 minutes). Baseline weight will be used to calculate dosing of CLN-619. Baseline weight is the weight obtained at screening. Dosing will be adjusted if there is a +/−10% change in weight.

In Module B, pembrolizumab 200 mg will be administered on Day 1 of each three-week treatment cycle as a 30-minute IV infusion (+10/−5 minutes). Please refer to the Pharmacy Manual for more information on pembrolizumab administration. When the CLN-619 is given in combination with pembrolizumab in Module B of this study, pre-medications including dexamethasone, will be dosed first followed by pembrolizumab. CLN-619 will be administered immediately following completion of pembrolizumab infusion (+30 minutes). In instances where an infusion related reaction occurs during dosing of pembrolizumab, administration of combination therapy may be delayed.

In Module C, standard of care chemotherapy should be administered prior to CLN-619 administration on Day 1 of each 3-week treatment cycle as per prescribing information or institutional policies. In Module D, CLN-619 (10 mg/kg) will be administered over 120 minutes on Day 1 of Cycle 1, first of the 3 weekly dose in Cycle 1. For all subsequent weekly doses (C1D8 and C1D15) and cycles (Cycle 2 and beyond), infusion can be administered over 60 minutes if the patient does not experience an IRR.

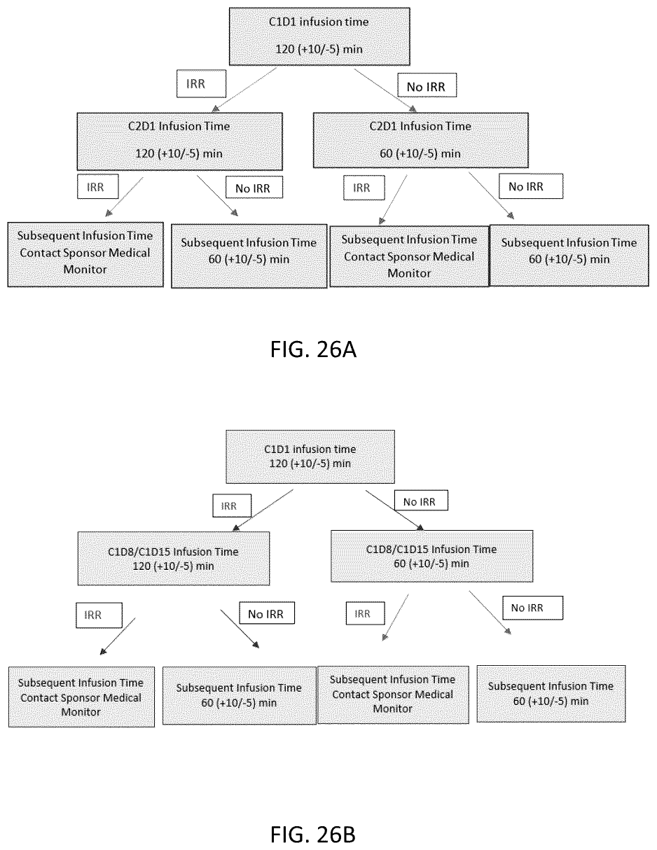

Prior to the administering CLN-619 pre-infusion medications including required corticosteroid will be administered. Infusion of CLN-619 will be administered on Cycle 1 Day 1 as a 120 (+10/−5) minute IV infusion. Baseline weight will be used to calculate dosing of CLN-619. If no infusion related reactions occur with the first cycle of treatment, then all subsequent three-week cycles will utilize a 60 (+10/−5) minute IV infusion. If an infusion related reaction occurs during the Cycle 1 Day 1 visit, the Cycle 2 Day 1 CLN-619 administration will utilize a 120 (+10/−5) minute IV infusion, followed by subsequent cycles utilizing a 60 (+10/−5) minute IV infusion (FIGS. 26A and 26B). If an IRR occurs beyond C2D1 visit, clinical sites should contact the Sponsor for guidance on future infusions.

### Pre-Medications for Infusion Related Reaction Prophylaxis

For patients to be treated with either CLN-619 alone or in combination with pembrolizumab or in combination with chemotherapy, pre-medications for infusion related reaction prophylaxis with corticosteroids, antihistamine, and/or antipyretics are mandatory. Suitable corticosteroids include dexamethasone; suitable antihistamines include diphenhydramine and famotidine; suitable antipyretics include paracetamol, acetaminophen, and ibuprofen. Folic acid supplements may also be administered.

### Grading and Management of Infusion Related Reactions

Infusion reactions may manifest with fever, chills, rigors, headache, rash, pruritis, arthralgias, hypo- or hypertension, bronchospasm, or other symptoms.

Further treatment recommendations are provided below in Table 8 and may be modified based on local treatment standards and guidelines as appropriate.

TABLE 8
Management of Infusion Related Reactions

| NCI CTCAE | | Premedication at |
|---|---|---|
| Grade | Treatment | subsequent dosing |
| Grade 1 | Remain at bedside and monitor patient until | Diphenhydramine 50 mg (or |
| Mild reaction: | recovery from symptoms. | equivalent) and/or |
| infusion | | paracetamol/acetaminophen |
| interruption | | 325 to 1000 mg at least 30 |
| not indicated, | | minutes before |
| intervention | | administration of CLN-619 |
| not indicated | | alone or in combination |
| | | with pembrolizumab. |
| Grade 2 | Stop the infusion of CLN-619 or | Prophylactic pre- |
| Requires | pembrolizumab and monitor symptoms | medications are |
| infusion | Begin an IV infusion of normal saline. | recommended for future |
| interruption | Diphenhydramine 50 mg IV (or | infusions: diphenhydramine |
| but responds | equivalent). | 50 mg (or equivalent) |
| promptly to | Paracetamol/acetaminophen 325 to | and/or |
| symptomatic | 1000 mg. | paracetamol/acetaminophen |
| treatment | Remain at bedside and monitor patient | 325 to 1000 mg or |
| (e.g., | until resolution of symptoms. | ibuprofen 400 to 600 mg |
| antihistamines, | Corticosteroid (hydrocortisone: 25-100 | administered once orally at |
| NSAIDS, | mg IV or Decadron: 10 mg IV or | least 30 minutes before |
| narcotics, IV | equivalent). | additional CLN-619 or |
| fluids); | Bronchodilator therapy may also be | pembrolizumab |
| prophylactic | administered as appropriate. | administration. |
| medications | Restart the infusion at 50% of the | Corticosteroids are |
| indicated | original infusion rate when symptoms | prohibited for subsequent |
| for ≤24 hrs | resolve; if no further complications | infusions unless approved |
| | ensue after 30 minutes, the rate may be | during discussion with the |
| | increased gradually to 100% of the | Sponsor's Medical |
| | original infusion rate. | Monitor. |
| | If symptoms recur, then no further | |
| | CLN-619 or pembrolizumab | |
| | (whichever was determined by the | |
| | Investigator to have caused the infusion | |
| | reaction) will be administered at that | |
| | visit. | |
| | Administer diphenhydramine 50 mg IV | |
| | and remain at bedside and monitor the | |
| | patient until resolution of symptoms. In | |
| | the event of an infusion related reaction | |
| | to pembrolizumab, administration of | |
| | CLN-619 may proceed at that visit if | |
| | deemed safe by the Investigator. | |
| | The amount of study drug infused must | |
| | be recorded on the eCRF. | |
| Grades 3 or 4 | Stop the infusion of CLN-619 or | No subsequent dosing |
| Grade 3: | pembrolizumab and monitor symptoms | |
| Prolonged | Immediately discontinue infusion of | |
| (i.e., not | CLN-619 or pembrolizumab. | |
| rapidly | Begin an IV infusion of normal saline. | |
| responsive to | Recommend bronchodilators. | |
| symptomatic | Epinephrine/adrenaline 0.2 to 1 mg of a | |
| medication | 1:1,000 solution for subcutaneous | |
| and/or brief | administration or 0.1 to 0.25 mg of a | |
| interruption of | 1:10,000 solution injected slowly for IV | |
| infusion); | administration. | |
| recurrence of | Diphenhydramine 50 mg IV. | |
| symptoms | Methylprednisolone 100 mg IV (or | |
| following | equivalent), as needed. | |
| initial | Patient should be monitored until the | |
| improvement; | Investigator is comfortable that the | |
| hospitalization | symptoms will not recur. | |
| indicated for | CLN-619, pembrolizumab or both will | |
| other clinical | be permanently discontinued. | |
| sequelae (e.g., | Investigators should follow their | |
| renal | institutional guidelines for the treatment | |
| impairment, | of anaphylaxis. | |
| pulmonary | Remain at bedside and monitor patient | |
| infiltrates) | until recovery from symptoms. | |
| Grade 4: | In the case of late-occurring | |
| Life- | hypersensitivity symptoms (e.g., | |
| threatening; | appearance of a localized or generalized | |
| pressor or | pruritis within one week after | |
| ventilatory | treatment), symptomatic treatment may | |
| support | be given (e.g., oral antihistamine, or | |
| indicated | corticosteroids). | |

### Tumor Imaging and Assessment of Disease Response by RECIST v 1.1

Images will be read locally by the Investigator, and efficacy response assessments are per Investigator assessment. Tumor imaging is strongly preferred to be acquired by CT with contrast. For the chest, abdomen and pelvis, contrast-enhanced MRI may be used when CT with iodinated contrast is contraindicated, or when mandated by local practice. MRI is the strongly preferred modality for imaging the brain, where brain imaging is clinically indicated. If MRI is medically contraindicated, CT with contrast is an acceptable alternative.

The same imaging technique regarding modality, ideally the same scanner, and the use of contrast should be used in a patient throughout the study to optimize the reproducibility of the assessment of existing and new tumor burden and improve the accuracy of the assessment of response or progression based on imaging.

Any patient that has completed 34 cycles of treatment will have tumor imaging performed at the Safety Follow-up visit. Patients that discontinued treatment prior to 34 cycles may not require tumor imaging at this visit if performed≤12 weeks prior to the visit.

### RECIST Assessment of Disease

RECIST v1.1 will be used as the primary measure for assessment of tumor response in this study and will be used to make treatment decisions.

Confirmatory scans for patients that have an objective response should be performed at least four weeks after the scan in which a response was first noted, or at next protocol required scan.

Clinical stability is defined as the following:
Absence of symptoms and signs indicating clinically significant progression of disease.
No requirements for intensified management, including increased analgesia, radiation, or other palliative care.
For more information on RECIST, refer to https://recist.eortc.org/recist-1-1-2/.

### Pharmacokinetics

Blood samples will be collected to assess CLN-619 and pembrolizumab (Module B only) serum concentration(s) and to determine relevant pharmacokinetic parameters.

### Immunogenicity

Blood samples will be collected to assess immunogenicity parameters, including human antidrug (CLN-619, and pembrolizumab (Module B only)) antibody titer (ADA).

### Definition of an AE

An AE is any untoward medical occurrence or worsening of a pre-existing medical condition in a clinical trial participant administered a medicinal product, and that does not necessarily have a causal relationship with this treatment.

An AE can therefore be:
Any unfavorable and unintended clinical sign (including an abnormal laboratory finding, for example).
Any symptom, disease or injury temporally associated with the use of the investigational product, whether or not related to the investigational product.

Aes will not include:
A medical or surgical procedure such as endoscopy, tooth extraction, or transfusion (although the condition that leads to the procedure may be an AE).
A pre-existing disease or condition present at the start of the study that does not worsen during the study.
Any situation where an untoward medical occurrence has not occurred (for example, hospitalizations for elective cosmetic surgery or social admissions).
A laboratory test result determined not to be clinically significant by the Investigator.

Adverse events can be spontaneously reported or elicited during open-ended questioning, examination, or evaluation of a patient. (In order to prevent reporting bias, patients should not be questioned regarding the specific occurrence of one or more Aes.)

Chronic illnesses which are present prior to study entry should be recorded in the medical history section of the eCRF and only be reported as Aes if there is an increase in the frequency or severity of the condition during the study.

Natural progression or deterioration of the malignancy under study will be recorded as part of the efficacy evaluation and should not be recorded as an AE/SAE. Death due to disease progression will be recorded as part of the efficacy evaluation and will not be regarded as an SAE.

Signs and symptoms of disease progression should not be reported as Aes/SAEs if they are clearly related to a relapse or an expected change related to disease progression of the baseline malignancy. These signs and symptoms should only be reported as Aes/SAEs (depending on the investigator's judgement) if they are:
Judged by the investigator to be unusually severe or an accelerated malignancy, or
If the investigator considers the deterioration of malignancy signs and symptoms to be caused directly by the IMP.

If there is any uncertainty about an AE being due solely to the malignancy under study, it should be reported as an AE/SAE as appropriate.

The Investigator, who is a qualified physician, and any designees are responsible for detecting, assessing, documenting, and reporting events that meet the definition of an AE or SAE, as well as other reportable safety events. Investigators remain responsible for following up Aes, SAEs and other reportable safety events for outcome.

### Severity Grading of Adverse Events

The severity of Aes will be recorded in accordance with the NCI CTCAE v5.0. This guidance provides a common language to describe levels of severity, to analyze and interpret data, to scale the aggregate AE score, and to articulate the clinical significance of all Aes. A copy of the current NCI CTCAE version can be downloaded at http://evs.nci.nih.gov/ftp1/CTCAE/About.html.

For Aes not listed in the NCI CTCAE, the following gradings should be used:
Grade 1: Mild AE—asymptomatic or mild symptoms; clinical or diagnostic observations only; intervention not indicated.
Grade 2: Moderate AE—minimal, local or non-invasive intervention indicated; limiting age-appropriate instrumental activities of daily living.
Grade 3: Severe AE—Severe or medically significant but not immediately life-threatening; hospitalization or prolongation of hospitalization indicated; disabling; limiting self-care activities of daily living.
Grade 4: Life-threatening or disabling AE—Life-threatening consequences; urgent intervention indicated.
Grade 5: Death related to AE

An AE that is assessed as severe is not the same as a serious AE. Severity is a category utilized for rating the intensity of an event and both Aes and SAEs can be assessed as severe. An AE is defined as ‘serious’ when it meets one of the pre-defined serious outcomes as described below.

### Sample Size

No formal sample size estimation was performed.

### Module A:

The number of patients in the dose escalation phase was estimated to be up to 50 across seven cohorts, including cohort extension in all dose-levels (45 patients have been enrolled). It is estimated that up to 240 patients may be enrolled in Module A Cohort Expansions (assuming full expansion). In total, it is estimated that up to approximately 285 patients will be dosed in Module A of this trial.

During dose escalation, patients that withdraw from treatment for reasons other than a DLT during the three-week DLT monitoring period will be replaced.

### Module B:

The number of patients in the combination dose escalation was estimated to be up to 30 patients (assuming six patients in up to five dose levels); 25 patients have been enrolled. It is estimated that up to 200 patients will be enrolled in Module B Cohort Expansion. In total, it is estimated that up to approximately 225 patients will be dosed in Module B of this trial.

### Module C:

The number of patients in the chemotherapy combination cohort will be up to 40 patients per indication (40 each for NSCLC, endometrial, and epithelial ovarian). In total, it is estimated that up to approximately 120 patients will be dosed in the Module C of this trial.

### Module D:

For Module D monotherapy loading dose cohort, up to 10 patients will be enrolled.

During the dose escalation cohorts, patients that withdraw from the study for reasons other than a DLT during the three-week DLT monitoring period will be replaced.

### Results:

64 patients received CLN-619 in combination with pembrolizumab (n=22) or as monotherapy (n=42). The baseline characteristics are presented in Table 9 below.

TABLE 9
Baseline characteristics

| | CLN-619 + | | CLN-619 | |
|---|---|---|---|---|
| | pembrolizumab | | monotherapy | |
| | (n = 22) | | (n = 42) | |
| Age, y, median (range) | 68.5 | (38, 82) | 62.5 | (26, 83) |
| Female, n (%) | 11 | (50.0) | 25 | (59.5) |
| ECOG PS 1, n (%) | 15 | (68.2) | 28 | (66.7) |
| Tumor type | | | | |
| NSCLC | 6 | (27.3) | 5 | (11.9) |
| Cervical | 4 | (18.2) | 5 | (11.9) |
| Ovarian | 3 | (13.6) | 3 | (7.1) |
| Prostate | 2 | (9.1) | 3 | (7.1) |
| Colorectal | 1 | (4.5) | 6 | (14.3) |
| Endometrial | 0 | | 3 | (7.1) |
| Other | 6 | (27.3)^{a} | 17 | (40.5)^{b} |
| Months since diagnosis, median (range) | 46.3 | (4, 160) | 37.4 | (9, 207) |
| No. of prior systemic therapies, median | 3 | (1, 8) | 3 | (1, 7) |
| (range) | | | | |
| Prior CPI therapy, n (%) | 9 | (40.9) | 21 | (50.0) |

CPI, checkpoint inhibitor; NSCLC, non-small cell lung cancer.
^{a}Other tumor types in the combination cohorts: gastric (2 patients), esophageal (1), head and neck (1), skin (1), and urothelial cancer (1);
^{b}Other tumor types in the monotherapy cohorts: breast (2), pancreatic (2), sarcoma (2), adenoid cystic carcinoma (1), caecal cancer (1), duodenum (1), head and neck (1), kidney (1), leiomyosarcoma (1), mediastinal intimal sarcoma (1), melanoma (1), parotid gland (1), peritoneal mesothelioma (1), and thyroid (1).
Of 22 patients treated with CLN-619 + pembrolizumab, 18 were RECIST-evaluable for response. 4 patients did not have post-baseline imaging for response evaluation due to withdrawal of consent (n = 2), death due to disease progression (n = 1), and transfer to hospice and acute kidney injury (n = 1). Confirmed responses (all PR) were observed in three patients treated with CLN-619 at doses ≥3 mg/kg in combination with pembrolizumab. Responses were observed in patients with tumor types not typically responsive to checkpoint inhibitor (CPI) alone.

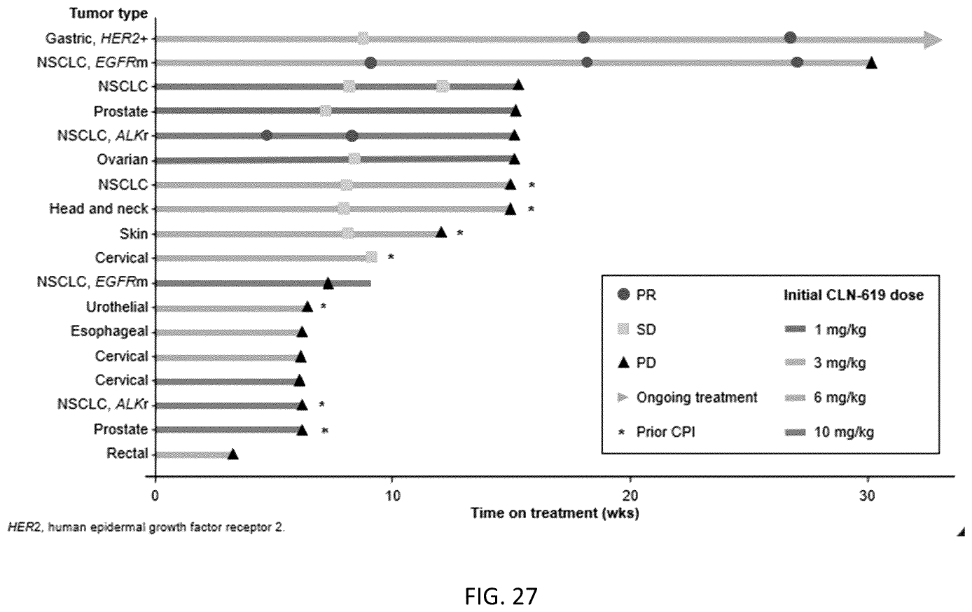

Characteristics of the responders are detailed in Table 10 and FIG. 27 depicts time on treatment and clinical activity for the combination cohorts.

TABLE 10
Characteristics of responders

| | Number | | CPI | | Duration |
|---|---|---|---|---|---|
| | of Prior | | Responsive | | of |
| | Lines of | Prior | Tumor | Best | Response |
| Tumor Type | Therapy | CPI | (Yes/No?) | Response | (weeks) |
| NSCLC, EGFR | 6 | No | No | PR | 24 |
| exon 18/21 | | | | | |
| NSCLC, ALKr | 2 | No | No | PR | 12.7 |
| Gastric, HER2+ | 3 | No | Yes | PR | 8.9 |
| | | | | | (ongoing) |

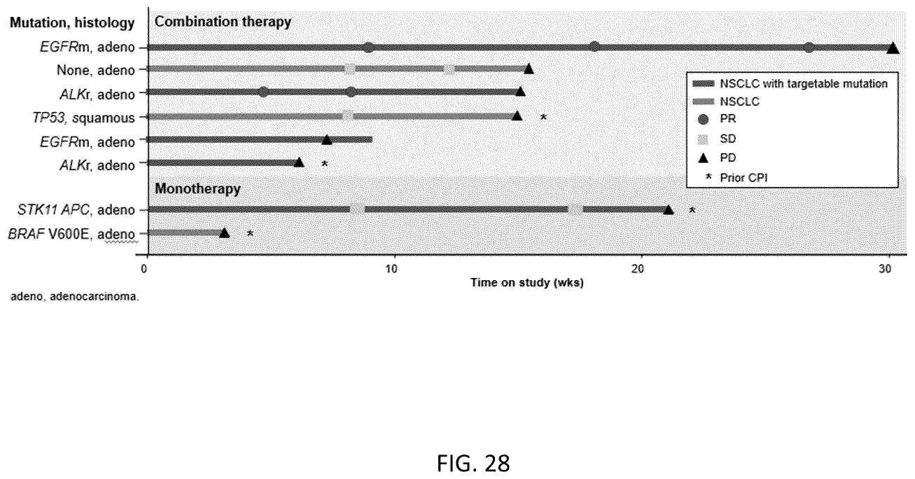

There was efficacy in NSCLC for both the monotherapy and combination cohorts, which is depicted in FIG. 28, which shows the time on treatment and clinical activity in patients with NSCLC. Objective responses and stable disease (SD) were observed in patients with NSCLC with targetable oncogenic mutations in the CLN-619 monotherapy and combination cohorts.

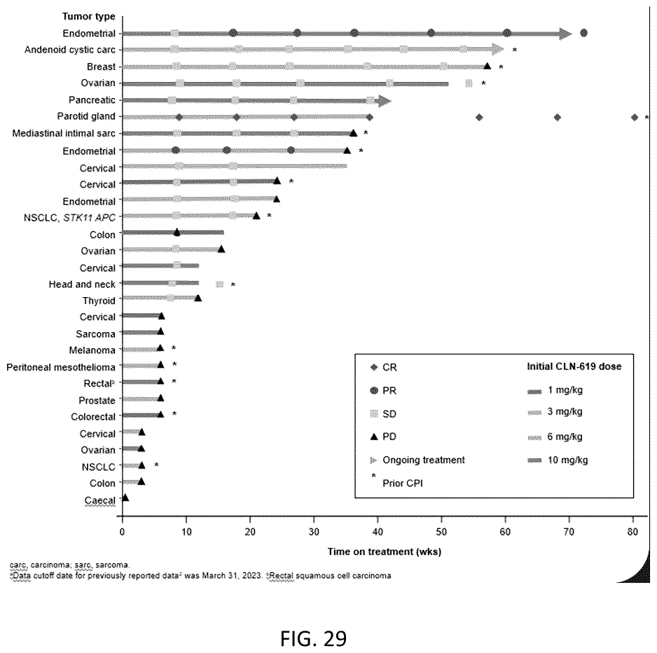

Among 42 patients treated with CLN-619 monotherapy, 29 received CLN-619 at a dose ≥1 mg/kg and were RECIST-evaluable. 6 patients did not undergo post-baseline imaging for response evaluation due to clinical progression (n=3), hospice (n=1), and withdrawal of consent (n=2). 7 patients received subtherapeutic doses (<1 mg/kg) or were not RECIST-evaluable for response. The clinical benefit rate (CBR) was 41.4% (1 complete response (CR), 2 PR, 9 SD ≥18 wks). The characteristics of patients with response or SD ≥18 wks is shown in Table 11 and the time on treatment and clinical activity for monotherapy cohorts ≥1 mg/kg is depicted in FIG. 29.

TABLE 11
Characteristics of patients with response or SD ≥18 wks

| | No. of prior | Best | |
|---|---|---|---|
| Tumor type | lines of therapy | response | DoR, wks |
| Responders (n = 3) | | | |
| Mucoepidermoid parotid | 2 | CR | 71 |
| Endometrial (serous, MMRp) | 5 | PR | 31 |
| Endometrial (endometrioid, MMRp) | 3 | PR | 55 |
| SD ≥18 wks (n = 9) | | | |
| Cervical squamous (n = 2); breast (ER/PR+, | Mean: 3.6 | SD ≥18 | Range: 18-56 |
| HER2−; n = 1); ovarian (n = 1); endometrial | Range: 1-7 | wks | |
| carcinosarcoma (n = 1); mediastinal intimal | | | |
| sarcoma (n = 1); adenoid cystic carcinoma | | | |
| (n = 1); pancreatic adenocarcinoma (KRAS | | | |
| G12V; n = 1); NSCLC (STK11m; n = 1) | | | |

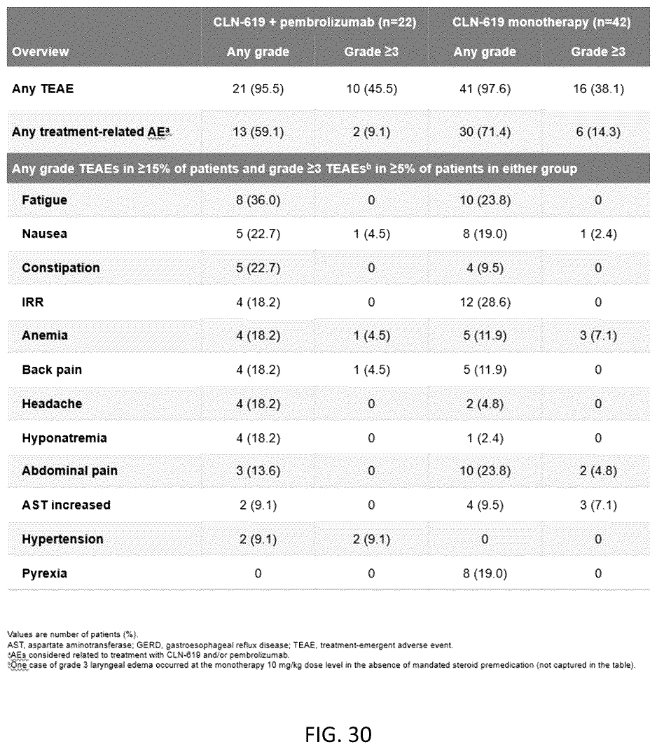

Safety: CLN-619 plus pembrolizumab combination therapy was well tolerated at doses ranging from 1 to 6 mg/kg and demonstrated clinical activity, including objective response, in tumor types typically unresponsive to pembrolizumab. No adverse effects (AEs) met protocol-defined DLT criteria and Most TEAEs were grade ½ (See FIG. 30). Treatment-related AEs (TRAEs) reported in ≥10% of patients were fatigue (combination: 18.2%; monotherapy: 9.5%) and IRRs (combination: 18.2%; monotherapy: 28.6%). The only grade ≥3 treatment-related AE reported in >5% of patients in any group was increased AST (combination: 0; monotherapy: 7.1%). TEAEs led to discontinuation of study treatment in 13.6% (3/22) of patients in the combination therapy cohorts and 9.5% (4/42) in the monotherapy cohorts. IRR is an emerging known AE with CLN-619 and IRR was the most frequently reported TRAE with CLN-619. With administration of prophylactic premedications, most IRRs were grade 1 or 2 and occurred on Day 1 of Cycle 1 and resolved quickly. There were no treatment-related deaths.

Conclusions: Objective responses were observed with CLN-619+pembrolizumab in patients with tumor types that are typically unresponsive to pembrolizumab (e.g., NSCLC with ALKr and EGFRm). CLN-619+pembrolizumab was well tolerated at doses ranging from 1 to 10 mg/kg. Longer term follow-up for patients treated with CLN-619 monotherapy confirms favorable safety and durable clinical benefit, including objective responses in multiple tumor types and in patients with disease progression after CPI therapy. Based on these findings, expansion cohorts are enrolling in endometrial cancer and NSCLC in monotherapy & combination cohorts. The best confirmed response for RECIST-evaluable patients is shown in Table 12.

TABLE 12
Best confirmed response (RECIST-evaluable patients)

| | CLN-619 + | CLN-619 monotherapy ≥1 | |
|---|---|---|---|
| | pembrolizumab (n = 18) | mg/kg (n = 29) | |
| ORR, n (%) | 3 (16.7) | 3 | (10.3) |
| CR | 0 | 1 | (3.4) |
| PR | 3 (16.7) | 2 | (6.9) |
| SD ≥18 wks | 0 | 9 | (31.0) |
| CBR (CR + PR + | 3 (16.7) | 12 | (41.4) |

SD ≥18 wks)

ALKr, anaplastic lymphoma kinase gene rearrangement; CBR, clinical benefit rate; CR, complete response; EGFRm, epidermal growth factor receptor mutation; PR, partial response; ORR, objective response rate; SD, stable disease.

CLN-619 has also demonstrated single-agent activity, including objective responses across multiple tumors.

### Example 4. Conclusions from Use of CLN-619 (Anti-MICA/B Antibody) in Solid Tumors and Rationale for Treatment of Multiple Myeloma

CLN-619 will also be studied in a Phase 1 clinical trial (NCT06381141) in patients with relapsed/refractory multiple myeloma.

### CLN-619 Shows Anti-Tumor Efficacy in Treatment of Advanced and Solid Tumors and is Well Tolerated as a Monotherapy and in Combination Therapy

In the previous examples, the inventors show clinical development of CLN-619, a humanized IgG1 monoclonal antibody that specifically binds the NKG2D ligands MICA and MICB, in patients with advanced solid tumors. Those studies show in vitro binding data that collectively supports the highly specific and potent binding of CLN-619 to MICA/MICB, whereby CLN-619 exhibited broad reactivity and high affinity binding to representative allelic variants of MICA and the canonical MICB allelic variant. CLN-619 has multiple modes of action. CLN-619 binds to and prevents the proteolytic release of MICA/MICB from the surface of tumor cells, augmenting NKG2D-mediated immune cell activation with antibody dependent cellular cytotoxicity (ADCC) and antibody dependent phagocytosis (ADCP) of MICA/MICB expressing tumor cells. Furthermore, in vivo studies demonstrated that CLN-619 treatment generates potent anti-tumor activity in mouse models bearing MICA/MICB-expressing human liver, lung, and multiple myeloma tumor xenografts over a dose range of 0.1 mg/kg to 10 mg/kg. Anti-tumor efficacy was also observed at doses as low as 0.1 mg/kg in the lung cancer model.

NCT05117476 described in the above examples, enrolled patients with advanced and metastatic solid tumors in a study of CLN-619 (CLN-619-001). Multiple doses of CLN-619 (maximum dose of 10 mg/kg) were tested, administered every three weeks either alone or in combination with pembrolizumab. In that study, CLN-619 was tolerated in both the monotherapy and combination therapy settings with no dose-limiting toxicities observed during dose escalation and no Grade 3 treatment related AEs (TRAE) in patients who have received protocol mandated pre-medications.

As of 22 Jun. 2023, 61 patients were exposed to intravenous doses of CLN-619 at 0.1, 0.3, 1, 3, 6, and 10 mg/kg, respectively on a 21-day cycling schedule. Forty-three were exposed to monotherapy of CLN-619 with 10 patients receiving the 3 mg/kg dose, 11 patients receiving the 6 mg/kg dose, and 12 patients receiving the 10 mg/kg dose. Eighteen patients were exposed to CLN-619 at 1, 3, 6, and 10 mg/kg in combination with pembrolizumab with 6 receiving the 10 mg/kg dose. CLN-619 was found to be well tolerated with no AEs meeting protocol defined DLT criteria, and no Grade 4 TRAEs.

Treatment emergent Serious Adverse Events (SAEs) were monitored following administration of CLN-619 in monotherapy and in combination with pembrolizumab. There was only one event of Infusion Related Reaction (IRR) with laryngeal edema was experienced by a patient at dose level 10.0 mg/kg and was considered related to CLN-619. This patient was not premedicated with corticosteroids. There was one non-serious Grade 3 rash reported in the monotherapy group that was considered possibly related. There were no serious adverse reactions to CLN-619 reported within the pembrolizumab combination therapy, but 1 SAE of acute kidney injury (AKI) but it was not related to CLN-619. The most common treatment emergent AEs (TEAE) to date in ≥5% of patients treated with monotherapy CLN-619 include IRR (23.3%), abdominal pain (18.6%), fatigue (18.6%), and pyrexia (18.6%).

The majority of IRRs y occurred during or immediately after the first dose of CLN-619; in 1 case the IRR occurred after the sixth dose; and one patient had IRR after both the first and second dose. There was also 1 event that occurred in a patient who received combination treatment with pembrolizumab. All patients recovered without sequalae. The treatment regimens recommend IRR premedication treatment, and as well as a longer infusion time for the first administration of CLN-619. Monitoring of immune-related adverse events such as immune-mediated colitis, hepatitis, pneumonitis, hypothyroidism, hyperthyroidism, adrenal insufficiency, hypophysitis, nephritis with renal dysfunction, myocarditis and uveitis also is recommended.

### Rational for Use of CLN-619 in Treatment of Multiple Myeloma

Multiple myeloma (MM) is a plasma-cell neoplasm that stems from the malignant transformation of antibody producing plasma cells in the bone marrow and is hallmarked by the presence of increasing quantities of abnormal monoclonal immunoglobulins (M-proteins) in the serum and urine. The overproduction of M-proteins can lead to lethal complications including bone destruction, hypercalcemia, anemia, renal damage and increased risk of infection.

Multiple myeloma (MM) accounts for about 10% to 18% of hematologic malignancies. It is a disease of the elderly, with an overall median age at diagnosis of approximately 70 years, and is slightly more common among men than women. Multiple myeloma has a 2 to 3 times higher incidence in African American patients compared to Caucasians, making it the most common hematologic malignancy in this ethnic group. The global incidence of MM is approximately 160,000 cases per year. In the United States (US), there are approximately 34,500 new cases of MM and about 13,000 deaths from MM each year. The frequency of MM is unevenly distributed geographically, with the highest incidence in Europe, North America, and Australia/New Zealand. Despite recent advancements, multiple myeloma remains incurable with the most frequent causes of death being disease progression, infection, and renal failure. The median overall survival (OS) for patients with MM ranges from 2 to more than 10 years with approximately 15% of patients dying within 2 years after diagnosis.

Treatment options for MM include a variety of therapies all working to control and eliminate multiple myeloma cells. These drugs include proteosome inhibitors, immune modulators, steroids, antibodies, BCMA-targeted therapies, and chemotherapy. Unfortunately, most MM patients experience sequential relapses and require multiple lines of therapy. Relapsed MM cells typically exhibit a more aggressive phenotype resulting in shorter durations of response and decreased survival rates. Therapies at this stage typically require a multifaceted approach to address the heterogeneous biological aspects of the disease and the full clinical spectrum of disease-related complications. Long-term disease control may be best achieved by combining therapies that have distinct mechanisms of action, especially those that target key components of the immune system. Thus, there is a significant need for new therapies for MM.

It has been well established that MM cells can shed MICA/MICB from their surface via proteolytic shedding to evade tumor cell surveillance. Loss of MICA/MICB from the cell surface enables tumor cells to evade immune cell recognition and destruction which is accompanied by immune cell dysregulation by immune cell surface internalization of NKG2D and impairment of NK and CD8+ T cell function and expansion. High concentrations of shed MICA (sMICA) have been observed in serum from patients across multiple tumor types and have been shown to correlate with poor survival including in MM where it is an independent prognostic factor of OS and PFS. MM patients with a known presence of sMICA have shown decreased levels of NKG2D on NK and CD8+ T cells as well as diminished NK cell cytotoxicity. Furthermore, shed MICB (sMICB) has been shown to contribute to an immunosuppressive microenvironment via polarizing macrophages to an M2 phenotype and expansion of myeloid derived suppressor cells (MDSCs) in the tumor microenvironment (TME). Preclinical data has validated the role of the MICA/MICB-NKG2D axis in immune surveillance, as overexpression of MICA in human tumor models resulted in delayed tumor growth in mice and increased survival. Additionally, NKG2D deficient mice, while developmentally normal, were defective in tumor surveillance in models of spontaneous malignancy.

Immunomodulatory drugs such as lenalidomide and pomalidomide can enhance the expression of MICA and CD155 through the binding of cereblon and downregulation of transcription factors IKZF1, IKZF3 and IRF4 in both MM cell lines and primary MM cells leading to degranulation of interferon gamma from NK cells. Therefore, upregulated MICA/MICB on the myeloma cells could be stabilized with the use of an anti-MICA/MICB stabilizing antibody, thus potentiating the elimination of the myeloma cells by NKG2D-expressing NK and T cells. Additionally, immunomodulatory agents such as lenalidomide have been reported to enhance NK cell activity by lowering the threshold required for NK cell activation by CD16A and other receptors. In a phase 2 study of lenalidomide refractory patients, pomalidomide treatment resulted in functional activation of both circulating and bone marrow infiltrating NK and T cells. These changes included increased NK and T cell numbers along with upregulation of activating receptors including NKG2D. In the present example, the inventors suggest that increasing MICA and MICB expression on malignant plasma cells and improving immune cell activation by combination of CLN-619 with immunomodulatory agents could further synergize these effects.

It is proposed that a therapeutic agent that effectively restores MICA/MICB expression on the surface of tumor cells is a compelling approach to reverse tumor-mediated immune suppression for treating cancer patients with multiple myeloma.

While immunotherapy provides improvements in overall survival and quality of life and has substantially altered the treatment landscape for patients with MM, most patients with advanced disease will still experience disease progression on or after multiple double or triple therapy combinations, pointing to an unmet need for patients with refractory disease. There is a strong rationale for modulating both innate and adaptive immune responses with CLN-619 monotherapy or CLN-619 in combination with pomalidomide+dexamethasone. The present example provides a planned combination regimen using an FDA approved MM standard of care with robust scientific rationale and clinical data to support their combinations with CLN-619 towards MM disease. The apoptotic activity induced by pomalidomide+dexamethasone could induce cellular stress and in turn increase expression of NKG2D ligands on MM cells. Further stabilization of these MICA and MICB ligands by CLN-619 could lead to increased immune cell recognition and potentiate ADCC, an important cytolytic mechanism mediated by NK cells. Combination of IMiDs with monoclonal antibodies that have similar Fc-functionality as CLN-619 have been shown to have synergistic effects in enhancing NK cell activity in the clinical setting. Furthermore, higher levels of sMICA have been correlated to a reduced overall survival of MM patients compared to sMICA negative MM patients. sMICA has been shown to have immunosuppressive effects in the tumor microenvironment, therefore CLN-619's ability to bind to sMICA/sMICB has the potential to help alleviate the NK and T-cell dysfunction resulting in improved anti-myeloma activity.

Thus, by virtue of their respective modes of action, the present example proposes treatment of multiple myeloma using CLN-619 alone, or CLN-619 in combination with pomalidomide+dexamethasone could potentiate anti-myeloma effects of innate and adaptive immunity. Preferably, the methods contemplated by this example provide effective treatment of relapsed/refractory multiple myeloma (R/R MM).

This example details an open-label, dose finding study designed to address the objectives of safety, tolerability, and efficacy of CLN-619 monotherapy and in combination with pomalidomide+dexamethasone for the treatment of R/R MM.

Part 1A of the study will use a two ascending dose design during a 3-week DLT observation period for CLN-619 monotherapy (Cycle 1), and a 3-week DLT observation period for CLN-619 combination therapy (Cycle 4 or earlier) in Part 1C. Implementation of a 3+3 cohort design in Part 1 will be used to identify an optimal dose of CLN-619. Monotherapy of CLN-619 in Part 1A will be examined first, and preliminary efficacy will be assessed prior to patients advancing into dose extension on monotherapy (Part 1B) or combination therapy (Part 1C). Clinical response data from the first 4 cycles or less of CLN-619 monotherapy in Part 1A will be used to determine patient participation in Part 1B or 1C. Patients with a very good partial response (VGPR) or better per international myeloma working group (IMWG) response criteria will continue on their assigned dose of CLN-619 monotherapy (Part 1B). Patients who have progressive disease (PD), partial response (PR), minimal response (MR), or stable disease (SD) per IMWG response criteria will continue to receive their assigned dose of CLN-619 in combination with pomalidomide+dexamethasone (Part 1C).

Based on the totality of the data of Part 1 reviewed by SRC, a cohort of combination CLN-619 with pomalidomide+dexamethasone starting from cycle 1 will be initiated in Part 2.

### Dose Rationale For Treating R/R Multiple Myeloma

A starting dose of 3 mg/kg Q3W of CLN-619 has been selected for this study based on the totality of safety, PK and biomarker (sMICA) data from the FIH study (CLN-619-001 outlined in Examples 2 and 3) in patients with advanced and metastatic solid tumors and in the relative abundance of the target in MM patients.

The studies of patients with locally advanced or metastatic solid tumors enrolled in the monotherapy arm of Study CLN-619-001 and exposed to at least one dose of CLN-619 monotherapy provide the rationale for dosing in MM. In the aforementioned study, the safety, efficacy, and PK of CLN-619 have been explored in a range of doses from 0.1 mg/kg to 10 mg/kg in patients with advanced solid tumors whose disease has progressed after available therapies. No DLTs have been observed in any patients. Only one patient experienced a TRAE (Grade 2 IRR with the first infusion of CLN-619) with no serious TRAEs in this dosed cohort.

Early observations to support monotherapy efficacy of CLN-619 in advanced or metastatic solid tumors (i.e., those of Examples 2 and 3 herein above) have been promising. Confirmed objective responses (RECIST v1.1) have been observed at dose levels ≥3 mg/kg: one CR in a patient with parotid gland cancer treated at the 3 mg/kg dose level and two PRs in endometrial cancer patients treated at the 3 and 10 mg/kg dose levels. Stable disease lasting for at least 6 cycles has also been observed at dose-levels of 1 mg/kg and above.

The preliminary PK of CLN-619 and pharmacodynamics of sMICA were also determined in Study CLN-619-001 (i.e., those of Examples 2 and 3 herein above). At steady-state (Cycle 3), exposure increased greater than dose proportional from 0.1 mg/kg to 1 mg/kg but were approximately dose proportional from 1 mg/kg to 10 mg/kg. The dose cohort mean half-life of CLN-619 ranged from 60.5 to 464 hours (2.5 to 19 days) with longer half-lives generally observed at the higher doses and associated with a dose-related reduction in systemic clearance likely a result of saturation of target mediated clearance (TMDD).

Dose-related increases in the fold change of serum sMICA concentrations were observed following treatment with CLN-619. The individual profiles of sMICA generally show a steady increase in serum concentrations with continued treatment with CLN-619, particularly at the higher dose levels. Binding of CLN-619 to sMICA results in elevated serum concentrations of total sMICA which likely leads to an overall reduction in free sMICA. As sMICA has immunosuppressive effects in the tumor microenvironment, lowering the concentrations of free sMICA may contribute to clinical activity of CLN-619. Saturation of TMDD and the pharmacodynamic response of sMICA indicate that near complete target saturation is achieved at dose levels beginning at 3 mg/kg.

Expression of MICA/MICB is often elevated in both solid tumors and multiple myeloma. sMICA serum concentrations also tend to be elevated in both solid tumor and multiple myeloma patients and higher concentrations have also been associated with poor prognosis. Some studies have reported higher serum concentrations of sMICA in multiple myeloma patients compared to solid tumor patients which may reflect an increase in the shedding rate and/or a higher overall tumor burden. Therefore, in order to achieve target saturation in MM patients, a dose of at least 3 mg/kg is recommended.

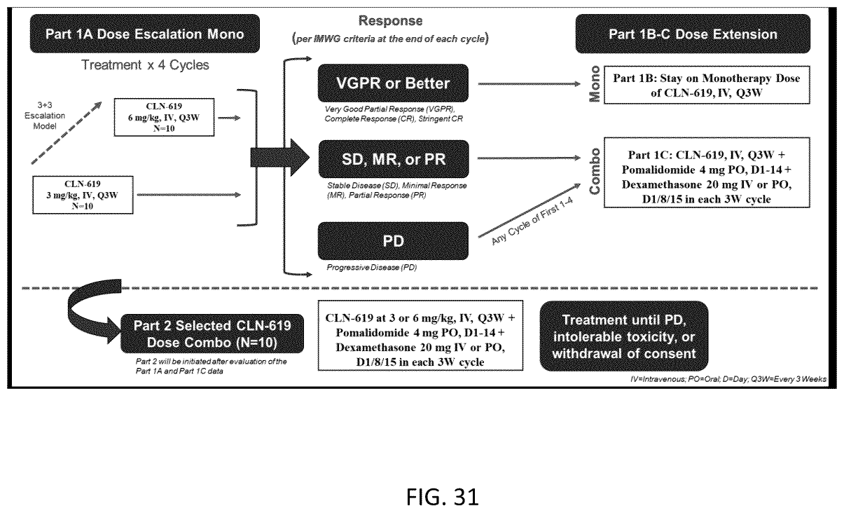

In summary, CLN-619 has been well tolerated in solid tumor patients up to 10 mg/kg where objective responses have only been observed at doses of 3 mg/kg or higher. Evidence of target saturation (both saturation of TMDD and pharmacodynamics of total sMICA in serum) appears to occur at approximately 3 mg/kg and higher for most patients. Therefore, the selected starting dose for CLN-619 of 3 mg/kg on a Q3W dosing schedule is expected to be both safe and effective target engagement in multiple myeloma patients. See FIG. 31 for overall study design.

### Treatment of R/R Multiple Myeloma Using Combination of CLN-619 and IMiDs.

Pomalidomide, marketed as Pomalyst® in the United States, is a thalidomide analogue or immunomodulatory (IMiD) agent manufactured by Bristol Myers Squibb for the treatment of MM. Pomalidomide when taken in combination with dexamethasone acts by inducing tumor cell apoptosis, enhancing T cell and NK cell mediated immunity and inhibiting pro-inflammatory cytokines.

A 4 mg standard of care dose is being utilized for this study, and dose reduction will be allowed to reduce neutropenia and pneumonia observed toxicities as seen in other studies.

FIG. 31 shows the overall study design for treatment of R/R MM. Since CLN-619 follows a 21-day dosing schedule, the inventors have designed a shorter dosing schedule for pomalidomide to match that of CLN-619. Pomalidomide standard of care dosing schedule is typically 28-days, with 21-days of dosing with one week off treatment. The current study design will give pomalidomide on days 1-14 with days 15-21 off the drug in each 21-day treatment cycle.

Patients will be treated with pomalidomide in combination with dexamethasone and CLN-619 until progressive disease, intolerable toxicity, or up to 2 years, whichever occurs first.

Dexamethasone is a corticosteroid used for treating a variety of diseases to suppress the immune system. Dexamethasone is usually used in combination with other drugs for multiple myeloma treatment.

Various doses of dexamethasone have been used in combination with pomalidomide, such as 20 mg (≥75 year old) or 40 mg (<75 years old) at weekly dosing, showing efficacy in R/R MM treatment.

In combination with pomalidomide and CLN-619, the current study design will give dexamethasone at 20 mg PO or IV on Day 1, 8, and 15 in each 21-day treatment cycle regardless of age.

Patients will be treated with dexamethasone in combination with CLN-619 and pomalidomide until progression of disease, intolerable toxicity, or up to 2 years, whichever comes first.

In the treatment cycle, at the Day 1 visit, patients who satisfy all eligibility criteria requirements will complete pre-dose assessments. After completing the pre-dose Day 1 assessments the first dose of the study treatment (CLN-619) will be administered under observation on site, after which post-dose assessments will be conducted. Study treatment, depending on each part and cohort will consist of an intravenous infusion of CLN-619.

In Part 1A (Dose Escalation) treatment will consist of the first 4 cycles of CLN-619 monotherapy. Cohorts of patients with R/R MM will be treated at ascending doses of CLN-619 monotherapy using a standard 3+3 dose escalation design. CLN-619 will be dosed at 3 and 6 mg/kg on a 21-day cycle. Preventive medication to reduce infusion related reactions is provided for Day 1 of the first and subsequent cycles of treatment.

Decision rules to guide dose escalation decisions and study conduct will be based on DLTs or MAD that occur in patients during the initial three weeks of CLN-619 monotherapy treatment. Any DLTs occurring in the subsequent cycles will be considered for recommended optimal dose decision-making.

A 3+3 design will be used to evaluate toxicity at each cohort. Upon completion of a 3+3 cohort (a minimum of three, maximum of six patients), if:
0 of 3 patients experience a DLT; dose escalation may proceed to the next cohort
1 of 3 patients experience a DLT; an additional three patients will be enrolled into the cohort
≥2 patients experience a DLT; the MTD has been exceeded, and further enrollment at that cohort will cease
Lower-dose cohorts may then be further explored until the MTD has been determined, including intermediate doses.

Patients unable to complete the DLT observation period for reasons other than experiencing a DLT will be excluded when estimating parameters, however, all patient data will be included in safety summaries and data listings.

Part 1B and 1C CLN-619 Dose Extension: Upon completion of the first four cycles of CLN-619 monotherapy treatment in Part 1A, patients that demonstrate acceptable safety and tolerability will proceed to Part 1B or 1C based on the outcome of their efficacy assessments.

Part 1B CLN-619 Monotherapy: Patients with a VGPR or better per IMWG response criteria after four cycles of treatment will continue their specific monotherapy treatment dose of CLN-619 at cycle 5 and beyond.

Part 1C CLN-619+Pomalidomide+Dexamethasone: Patients who have PR, MR, or SD per IMWG response criteria after four cycles of CLN-619 monotherapy will receive treatment of CLN-619 in combination with pomalidomide+dexamethasone starting from Cycle 5. Patients who have PD per IMWG criteria at any time upon completion of cycles 1~4 will receive treatment of CLN-619 in combination with pomalidomide+dexamethasone. A DLT observation period will occur during the first cycle (Day 1 to 21) of combination treatment. CLN-619 will be dosed on a 21-day cycle. Pomalidomide will be administered orally at 4 mg per day on Days 1 through 14 of repeated 21-day cycle starting on Cycle 4 Day 1. Dexamethasone will be administered orally or intravenously at 20 mg on Days 1, 8, and 15 of each cycle. Patients in Part 1C will be closely monitored for DLTs during the initial three-week cycle of combination treatment.

At any time during the study in Part 1B or 1C, if >30% of patients experienced AEs meeting the DLT criteria during the defined DLT observation period and beyond, or if safety findings are suggestive of clinically significant cumulative toxicity, the SRC may be convened to review data from the extension cohorts and to make recommendations regarding ongoing dosing of patients.

Part 2: Upon review of the available safety and efficacy data from Part 1, a single dose of CLN-619 in combination with pomalidomide+dexamethasone will be selected to enroll patients in Part 2 where CLN-619 in combination with pomalidomide+dexamethasone will be dosed starting from the first cycle of treatment. CLN-619 will be dosed on a 21-day cycle. Pomalidomide will be administered orally at 4 mg per day on Days 1 through 14 of repeated 21-day cycle. Dexamethasone will be administered orally or intravenously at 20 mg on Days 1, 8, and 15 of each cycle. At any time during the study in Part 2, if >30% of patients experienced AEs meeting the DLT criteria, or if safety findings are suggestive of clinically significant cumulative toxicity, the SRC may be convened to review data from the extension cohorts and to make recommendations regarding ongoing dosing of patients.

### REFERENCES

Acebes-Huerta A, Lorenzo-Herrero S, Folgueras A R, Huergo-Zapico L, Lopez-Larrea C, López-Soto A, Gonzalez S. Drug-induced hyperploidy stimulates an antitumor N K cell response mediated by NKG2D and DNAM-1 receptors. Oncoimmunology. 2015 Aug. 12; 5 (2): e1074378. doi: 10.1080/2162402X.2015.1074378. PMID: 27057443; PMCID: PMC4801427.
de Biasi A R, Villena-Vargas J, Adusumilli P S. Cisplatin-induced antitumor immunomodulation: a review of preclinical and clinical evidence. Clin Cancer Res. 2014 Nov. 1; 20 (21): 5384-91. doi: 10.1158/1078-0432.CCR-14-1298. Epub 2014 Sep. 9. PMID: 25204552; PMCID: PMC4216745.
Brahmer J R, Lacchetti C, Schneider B J, et al. Management of immune-related adverse events in patients treated with immune checkpoint inhibitor therapy: American Society of Clinical Oncology Clinical Practice Guideline. Journal of Clinical Oncology. 2018 Jun. 10; 36 (17): 1714-1768.
Bridge J A, Lee J C, Daud A, Wells J W, Bluestone J A. Cytokines, Chemokines, and Other Biomarkers of Response for Checkpoint Inhibitor Therapy in Skin Cancer. Front in Med. 2018; 5 (341): 1-18.
Capellero S, Erriquez J, Melano C, Mesiano G, Genta S, Pisacane A, Mittica G, Ghisoni E, Olivero M, Di Renzo M F, Aglietta M, Sangiolo D, Valabrega G. Preclinical immunotherapy with Cytokine-Induced Killer lymphocytes against epithelial ovarian cancer. Sci Rep. 2020 Apr. 15; 10 (1): 6478. doi: 10.1038/s41598-020-63634-z. PMID: 32296104; PMCID: PMC7160190.
Chitadze G, Bhat J, Lettau M, Janssen O, Kabelitz D. Generation of soluble NKG2D ligands: Proteolytic cleavage, exosome secretion and functional significance. Scandinavian Journal of Immunology. 2013; 78:120-129.
Cho H, Chung J Y, Kim S, Braunschweig T, Kang T H, Kim J, Chun E J, Hewitt S M, Kim J H. MICA/B and ULBP1 NKG2D ligands are independent predictors of good prognosis in cervical cancer. BMC Cancer. 2014; 14:957.
Di Modica M, Sfondrini L, Regondi V, Varchetta S, Oliviero B, Mariani G, Bianchi G V, Generali D, Balsari A, Triulzi T, Tagliabue E. Taxanes enhance trastuzumab-mediated ADCC on tumor cells through NKG2D-mediated N K cell recognition. Oncotarget. 2016 Jan. 5; 7 (1): 255-65. doi: 10.18632/oncotarget.6353. PMID: 26595802; PMCID: PMC4807996.
Dunai C and Murphy W J. N K cells for PD-1/P D-L1 blockade immunotherapy: pinning down the N K cell. J Clin Invest. 2018 Oct. 1; 128 (10): 4251-4253.
Frazao, A, Rethacker L, Messaoudene M, Avril M F, Toubert A, Dulphy N, Caignard A. NKG2D/NKG2-Ligand Pathway Offers New Opportunities in Cancer Treatment. Front Immunol. 2019; 10:661.
Friese M A, Platten M, Lutz S Z, Naumann U, Aulwurm S, Bischof F, Dichgans J, Buhring H J, Ramnesee H G, Steinle A, Weller M. MICA/NKGD-Mediated Immunogene Therapy of Experimental Gliomas. Can Res. 2003; (63) 8996-9006.
Gameiro S R, Caballero J A, Hodge J W. Defining the molecular signature of chemotherapy-mediated lung tumor phenotype modulation and increased susceptibility to T-cell killing. Cancer Biother Radiopharm. 2012 February; 27 (1): 23-35. doi: 10.1089/cbr.2012.1203. Epub 2012 Feb. 8.
Gao Q, Wang S, Chen X, Cheng S, Zhang Z, Li F, Huang L, Yang Y, Zhou B, Yue D, Wang D, Cao L, Maimela N R, Zhang B, Yu J, Wang L, Zhang Y. Cancer-cell-secreted CXCL11 promoted CD8⁺ T cells infiltration through docetaxel-induced-release of HMGB1 in NSCLC. J Immunother Cancer. 2019 Feb. 11; 7 (1): 42. doi: 10.1186/s40425-019-0511-6. PMID: 30744691; PMCID: PMC6371476.
Guerra N, Tan X Y, Joncker N T, Choy A, Gallardo F, Xiong N, Knoblaugh S, Cado D, Greendberg N R, Raulet D H. NKG2D-deficient mice are defective in tumor surveillance in models of spontaneous malignancy. Immunity. 2008; (28): 571-580.
Holdenrieder S, Stieber P, Peterfi A, Nagel D, Steinle A, Salih H R. Soluble MICA in malignant disease. Int. J. Cancer. 2006; 118:684-687.
Lanier L L. NKG2D Receptors and Ligands in Host Defense. Cancer Immunol Res. 2015; 3 (6) 575-582.
Liu H, Wang S, Xin J, Wang J, Yao C, Zhang Z. Role of NKG2D and its ligands in cancer immunotherapy. Am J Cancer Res. 2019; 9 (10): 2064-2078.
Lombana T N, Matsumoto M L, Bevers J III, Berkley A M, Toy E, Cook R, Gan Y, Du C, Liu P, Schnier P, Sandoval W, Ye Z, Scharnter J M, Kim J, Spiess C. High-resolution glycosylation site-engineering method identifies MICA epitope critical for shedding inhibition activity of anti-MICA antibodies. MABS. 2019 11 (1) 75-93.
Maccalli C, Giannarelli D, Chiarucci C, et al. Soluble NKG2D ligands are biomarkers associated with the clinical outcome to immune checkpoint blockade therapy of metastatic melanoma patients. Oncoimmunology. 2017 May 8; 6 (7): e1323618.
Okimoto T, Kotani H, Iida Y, Koyanagi A, Tanino R, Tsubata Y, Isobe T, Harada M. Pemetrexed sensitizes human lung cancer cells to cytotoxic immune cells. Cancer Sci. 2020 June; 111 (6): 1910-1920. doi: 10.1111/cas.14401. Epub 2020 Apr. 22. PMID: 32232903; PMCID: PMC7293070.
Okita R, Maeda A, Shimizu K, Nojima Y, Saisho S, Nakata M. Effect of platinum-based chemotherapy on the expression of natural killer group 2 member D ligands, programmed cell death-1 ligand 1 and HLA class I in non-small cell lung cancer. Oncol Rep. 2019 August; 42 (2): 839-848. doi: 10.3892/or.2019.7185. Epub 2019 Jun. 3. PMID: 31173242.
Schaer D A, Geeganage S, Amaladas N, Lu Z H, Rasmussen E R, Sonyi A, Chin D, Capen A, Li Y, Meyer C M, Jones B D, Huang X, Luo S, Carpenito C, Roth K D, Nikolayev A, Tan B, Brahmachary M, Chodavarapu K, Dorsey F C, Manro J R, Doman T N, Donoho G P, Surguladze D, Hall G E, Kalos M, Novosiadly R D. The Folate Pathway Inhibitor Pemetrexed Pleiotropically Enhances Effects of Cancer Immunotherapy. Clin Cancer Res. 2019 Dec. 1; 25 (23): 7175-7188. doi: 10.1158/1078-0432.CCR-19-0433. Epub 2019 Aug. 13. PMID: 31409612.
Schmiedel D, and Mandelboim O. NKG2D Ligands-Critical Targets for Cancer Immune Escape and Therapy. Front Immunol. 2018 9:2040.
Sheppard S, Ferry A, Guedes J, 1 Guerra N. The Paradoxial role of NKG2D in Cancer Immunity. Front. in Immun. 2018; 9:1808.
Sheppard S, Guedes J, Mroz A, Zavitsanou A, Kudo H, Rothery S M, Angelopoulos P, Goldin R, Guerra N. The immunoreceptor NKG2D promotes tumour growth in a model of hepatocellular carcinoma. Nat Commun. 2017; (8): 13930.
Wanderley C W, Colón D F, Luiz JPM, Oliveira F F, Viacava P R, Leite C A, Pereira J A, Silva C M, Silva C R, Silva R L, Speck-Hernandez C A, Mota J M, Alves-Filho J C, Lima-Junior R C, Cunha T M, Cunha F Q. Paclitaxel Reduces Tumor Growth by Reprogramming Tumor-Associated Macrophages to an M1 Profile in a TLR4-Dependent Manner. Cancer Res. 2018 Oct. 15; 78 (20): 5891-5900. doi: 10.1158/0008-5472.CAN-17-3480. Epub 2018 Aug. 13. PMID: 30104241.
Wensveen F M, Jelencic V, Polic B. NKG2D: A Master Regulator of Immune Cell Responsiveness. Front Immunol. 2018:9: 441.
Wu X, Feng Q M, Wang Y, Shi J, Ge H L, Di W. The immunologic aspects in advanced ovarian cancer patients treated with paclitaxel and carboplatin chemotherapy. Cancer Immunol Immunother. 2010 February; 59 (2): 279-91. doi: 10.1007/s00262-009-0749-9. Epub 2009 Aug. 30. PMID: 19727719; PMCID: PMC11030086.
Xiao G, Wang X, Sheng J, Lu S, Yu X, Wu J D. Soluble NKG2D ligand promotes MDSC expansion and skews macrophage to the alternatively activated phenotype. J Hematol Oncol. 2015; (8): 1-10.
Zhang T, Barder A, Sentman C L. Generation of Antitumor Responses by Genetic Modification of Primary human T cells with a Chimeric NKG2D Receptor. Can Res. 2006; (11): 5927-5933.
Zhao Y, Chen N, Yu Y, Zhou L, Niu C, Liu Y, Tian H, Lv Z, Han F and Cui J. Prognostic value of MICA/B in cancers: a systematic review and meta-analysis. Oncotarget. 2017; 8 (56): 96384-96395.
Zingoni A, Molfetta R, Fionda C, Soriani A, Paolini R, Cippitelli M, Cerboni C, Santoni A. NKG2D and Its Ligands: “One for All, All for One. Front Immunol. 2018; 9:476.

All features disclosed in the specification, including the claims, abstracts, drawings, and appendices, and all the steps in any method or process disclosed, may be combined in any combination, except combinations where at least some of such features and/or steps are mutually exclusive. Each feature disclosed in the specification, including the claims, abstract, drawings, and appendices, can be replaced by alternative features serving the same, equivalent or similar purpose, unless expressly stated otherwise. Thus, unless expressly stated otherwise, each feature disclosed is one example only of a generic series of equivalent or similar features.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. A method of treating cancer in a subject, the method comprising administering to the subject a therapeutically effective amount of a monoclonal antibody or antigen-binding fragment thereof, comprising light chain complementarity determining regions (LCDR) 1-3 and heavy chain complementarity determining regions (HCDR) 1-3 as follows:
LCDR1 is SEQ ID NO: 1, LCDR2 is SEQ ID NO: 2, LCDR3 is SEQ ID NO: 3, HCDR1 is SEQ ID NO: 4, HCDR2 is SEQ ID NO: 5, and HCDR3 is SEQ ID NO: 6.

2. The method of claim 1, wherein the subject has previously been treated with a PD-1 inhibitor an EGFR inhibitor, a VEGF inhibitor hormonal therapy, or chemotherapy.

3. (canceled)

4. (canceled)

5. The method of claim 1, wherein the cancer is endometrial cancer and the endometrial cancer is HER2+, is ER+, is PR+, or both ER+ and PR+; or the cancer is parotid cancer and the parotid cancer overexpresses one or both of EGFR and HER2 or the parotid cancer is a mucoepidermoid parotid cancer;
or the cancer is mutant for one or more of: EGFR, TP53, HER2, PTE¬¬N, ARID1A, CTNNB1, or PLK3R1.

6-11. (canceled)

12. The method of claim 2, wherein the subject responded to the VEGF inhibitor, the EGFR inhibitor or the hormone therapy, wherein the subject had a period of response of at least 1 month, and the administration of the monoclonal antibody or antigen-binding fragment thereof is after the progression following the period of response to the VEGF inhibitor, the EGFR inhibitor, or the hormone therapy;
or the subject had stable disease upon administration of the VEGF inhibitor, the EGFR inhibitor or the hormone therapy, and the subject had a period of stable disease of at least 1 month and the treatment with the VEGF inhibitor, the EGFR inhibitor, or the hormone therapy ceased before initiation of administration of the monoclonal antibody or antigen-binding fragment thereof.

13-20. (canceled)

21. The method of claim 1, wherein the EGFR inhibitor or the VEGF inhibitor comprises a small molecule tyrosine kinase inhibitor (TKI), or the hormone therapy comprises an aromatase inhibitor (AI) (e.g., anastrozole), a selective estrogen receptor modulator (SERM), a leuteinizing hormone-releasing hormone (LHRH) agonist, an anti-androgen, a CYP17 inhibitor, a progestin, an adrenolytic, or an estrogen receptor antagonist.

22. (canceled)

23. (canceled)

24. A method of claim 2 wherein:
(i) the subject responded to the PD-1 inhibitor; or
(ii) the subject had stable disease upon administration of the PD-1 inhibitor.

32. (canceled)

33. (canceled)

34. The method of claim 24, wherein the PD-1 inhibitor comprises pimivalimab, pembrolizumab, nivolumab, cemiplimab, AMP-224, APM-514, or spartalizumab.

35. (canceled)

36. The method of claim 1 further comprising:
administering to the subject a therapeutically effective amount of a corticosteroid prior to a first dose of the anti-MICA/B antibody.

37. The method of claim 36, wherein the corticosteroid is administered 30-60 minutes prior to administration of the monoclonal antibody or antigen-binding fragment thereof, and/or wherein the corticosteroid is administered at a dose of between about 2 mg to about 50 mg.

38-43. (canceled)

44. The method of claim 1, wherein the cancer parotid gland cancer, a cervical cancer, an endometrial cancer, a breast cancer, a colon cancer, an ovarian cancer, a prostate cancer, a sarcoma, a melanoma, an adenoid cystic salivary tumor, a peritoneal mesothelioma, a squamous cell carcinoma of the rectum, a leiomyosarcoma, a colorectal cancer, a kidney cancer, a thyroid cancer, an NSCLC, a duodenum cancer, a pancreatic cancer, a mediatinal intimal sarcoma, a head and neck cancer, or a caecal cancer or is a tumor of the female genital tract, a salivary gland tumor, a breast cancer, a prostate cancer, a lung cancer, or a colon cancer.

45-49. (canceled)

50. The method of claim 1, wherein the monoclonal antibody or antigen-binding fragment thereof is administered at a dose of about 3 mg/kg to about 10 mg/kg.

51. The method of claim 1, wherein the monoclonal antibody or antigen-binding fragment thereof is administered to the subject according to a dosing interval wherein the dosing interval comprises a three-week cycle, and wherein the anti-MICA/B antibody is administered once every three weeks (Q3W).

52-54. (canceled)

55. The method of claim 1, wherein the monoclonal antibody or antigen-binding fragment thereof comprises a light chain variable region (VL) comprises the amino acid sequence of SEQ ID NO: 7 and and a heavy chain variable region (VH) comprising the amino acid sequence of SEQ ID NO: 8.

56-59. (canceled)

60. The method of claim 1, wherein the monoclonal antibody or antigen-binding fragment thereof specifically binds to a MICA protein, a MICB protein, or both MICA and MICB protein.

61. (canceled)

62. The method of claim 60, wherein the MICA protein is membrane-bound MICA protein, soluble MICA protein, or both, and/or the MICB protein is membrane-bound MICB protein, soluble MICB protein, or both.

63. (canceled)

64. The method of claim 70, wherein the monoclonal antibody or antigen-binding fragment thereof is selected from a whole immunoglobulin, an scFv, a Fab, a F(ab′)2, or a disulfide linked Fv.

65. The method of claim 70, wherein the monoclonal antibody or antigen-binding fragment thereof is an IgG or IgM.

66. The method of claim 70, wherein the monoclonal antibody or antigen-binding fragment thereof is a humanized or a chimeric antibody.

67. The method of claim 1, wherein the method further comprises administering to the subject an effective amount of a PD-1 inhibitor.

68. (canceled)

69. The method of claim 67, wherein the anti-PD-1 inhibitor comprises pimivalimab, pembrolizumab, nivolumab, cemiplimab, AMP-224, APM-514, or spartalizumab.

70. A method of treating multiple myeloma in a subject, the method comprising administering to the subject a therapeutically effective amount of a monoclonal antibody or antigen-binding fragment thereof, comprising light chain complementarity determining regions (LCDR) 1-3 and heavy chain complementarity determining regions (HCDR) 1-3 as follows:
LCDR1 is SEQ ID NO: 1, LCDR2 is SEQ ID NO: 2, LCDR3 is SEQ ID NO: 3, HCDR1 is SEQ ID NO: 4, HCDR2 is SEQ ID NO: 5, and HCDR3 is SEQ ID NO: 6.

71. The method of claim 70, further comprising administering to said subject a therapeutically effective amount of an immunomodulatory drug.

72. The method of claim 71, further comprising administering to said subject a therapeutically effective amount of dexamethasone.

73. The method of claim 70, wherein after administration of the monoclonal antibody or antigen-binding fragment thereof, the subject has a response or stable disease.

74. The method of claim 70, wherein said subject has relapsed/refractory multiple myeloma.

75. The method of claim 70, wherein said subject is non-responsive to corticosteroids, melphalan, or a combination of vincristine, doxorubicin, dexamethasone.

76. The method of claim 70, further comprising administering to the subject a therapeutically effective amount of a corticosteroid prior to the first dose of the monoclonal antibody or antigen-binding fragment thereof.

77. The method of claim 76, wherein the corticosteroid is administered 30-60 minutes prior to administration of the monoclonal antibody or antigen-binding fragment thereof.

78. The method of claim 76, wherein the corticosteroid is administered at a dose of between about 2 mg to about 50 mg.

79. (canceled)

80. The method of claim 76, wherein the corticosteroid is administered orally or intravenously.

81. The method of claim 76, wherein the method further comprises administering one or more subsequent doses of the monoclonal antibody or antigen-binding fragment thereof.

82. The method of claim 81, wherein the corticosteroid is administered prior to the first dose of the monoclonal antibody or antigen-binding fragment thereof but is not administered prior to the one or more subsequent doses of the monoclonal antibody or antigen-binding fragment thereof.

83. (canceled)


SEQUENCE LISTING (12 sequences)

SEQ ID NO: 1 - protein, 16 aa, synthetic construct
  source 1..16: note = 3F9H1L3L Light Chain CDR1
RSSKSLLHSNLNTYLY

SEQ ID NO: 2 - protein, 7 aa, synthetic construct
  source 1..7: note = 3F9H1L3L Light Chain CDR2
RMSNLAS

SEQ ID NO: 3 - protein, 9 aa, synthetic construct
  source 1..9: note = 3F9H1L3L Light Chain CDR3
MQHLEYPFT

SEQ ID NO: 4 - protein, 10 aa, synthetic construct
  source 1..10: note = 3F9H1L3L Heavy Chain CDR1
GFTFSNYAMS

SEQ ID NO: 5 - protein, 17 aa, synthetic construct
  source 1..17: note = 3F9H1L3L Heavy Chain CDR2
YISPGGDYIYYADSVKG

SEQ ID NO: 6 - protein, 12 aa, synthetic construct
  source 1..12: note = 3F9H1L3L Heavy Chain CDR3
DRRHYGSYAMDY

SEQ ID NO: 7 - protein, 113 aa, synthetic construct
  source 1..113: note = 3F9H1L3L Light Chain Variable domain
DIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIKR

SEQ ID NO: 8 - protein, 121 aa, synthetic construct
  source 1..121: note = 3F9H1L3L Heavy Chain Variable domain
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSS

SEQ ID NO: 9 - protein, 219 aa, synthetic construct
  source 1..219: note = 3F9H1L3L Light Chain
DIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 450 aa, synthetic construct
  source 1..450: note = 3F9H1L3L Heavy Chain
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC